(19) United States
(12) Patent Application Publication
(10) Pub. No.: US 2026/0151372 A1
(43) Pub. Date: Jun. 4, 2026

(54) AZOTOMYCIN PRODRUGS WITH PREFERENTIAL TUMOR DELIVERY

(71) Applicant: The Johns Hopkins University, Baltimore, MD (US)

(72) Inventors: Rana RAIS, West Friendship, MD (US); Barbara SLUSHER, Kingsville, MD (US); Jesse ALT, Nottingham, MD (US); Lukas TENORA, Baltimore, MD (US)

(21) Appl. No.: 19/122,921

(22) PCT Filed: Nov. 2, 2023
(86) PCT No.: PCT/US2023/036643

§ 371 Date: Apr. 21, 2025

Related U.S. Application Data
(60) Provisional application No. 63/382,036, filed on Nov. 2, 2022.

(51) Int. Cl. A61K 31/4045; A61K 31/16; C07C 229/26; C07D 209/20

(52) U.S. Cl. CPC A61K 31/4045; A61K 31/16; C07C 229/26; C07D 209/20

(57) ABSTRACT

Disclosed are azotomycin prodrugs their use in treating diseases, disorders, or conditions associated with excess and/or aberrant glutamine utilization, including cancer.

### STATEMENT OF GOVERNMENTAL INTEREST

This invention was made with government support under grant CA229451 awarded by the National Institutes of Health. The government has certain rights in the invention.

### BACKGROUND

Azotomycin is a diazo analog of L-glutamine isolated from Streptomyces ambofaciens. It is a potent glutamine antagonist and broadly blocks glutamine utilizing reactions critical for the synthesis of nucleic acids, amino acids, proteins and the generation of alpha-ketoglutarate for energy metabolism. Azotomycin has shown robust efficacy in multiple preclinical cancer models and exploratory clinical studies. Although promising, its development was halted due to toxicity including emesis, anorexia, bloody feces, hepatotoxicity, nephrotoxicity, and CNS toxicities (tremors, convulsions, ataxia, lethergy).

### SUMMARY

In some aspects, the presently disclosed subject matter provides a compound of formula (I):

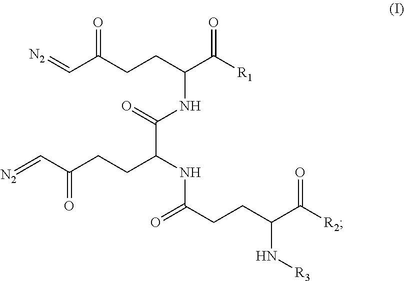

wherein:
R₁ and R₂ are each independently selected from —OR₄, —NR₅R₆, —O⁻M⁺, and —O—(CH₂CH₂O)ₙ—R₇, wherein:
n is an integer selected from 1, 2, 3, and 4;
R₄ is selected from H and C₁-C₄ substituted or unsubstituted branched or unbranched alkyl;
R₅ and R₆ are each independently H, C₁-C₄ substituted or unsubstituted branched or unbranched alkyl, -AA-COOR₇, wherein R₇ is C₁-C₄ substituted or unsubstituted branched or unbranched alkyl, and AA is an amino acid;
M⁺ is a metal cation, including, but not limited to Na⁺, K⁺, Li⁺, and, in some embodiments, NH₄⁺;
R₃ is selected from H, —(C═O)—R₈, —C(═O)—CH₂—(NH—C(═O)—CH₂—NR₉R₁₀)—CH₂—R₁₁, —C(═O)—CH—(NH—C(═O)—CH(NH₂)—CH—(CH₃)₂)—((CH₂)₃NH—C(═O)NH₂, —C(═O)—CH((CH₂)₄—NH—C(═O)—CH₃)(NH—C(═O)-adamantane), —C(═O)-AA-R₁₂, acetyl (Ac), dimethylglycine, dimethylglycyl (DMG), L-Lys(Ac), D-Val-L-Leu-L-Lys(Ac), D-Val-L-Leu-L-Lys(H), D-Val-L-Leu-L-Lys(DMG), L-Val-L-Cit, para-aminobenzoic acid (PABA), L-Cit-PABA, L-Val-L-Cit-PABA, Val-Cit-OH, dimethylglycyl-L-tryptophan, acetoxymethyl pivalate, 1-acetoxyethyl pivalate, acetoxy(phenyl)methyl pivalate, and 2-acetoxypropan-2-yl pivalate; wherein AA is an amino acid, wherein the amino acid of R₃, R₅, and R₆ can be selected from an amino acid or amino acid-related substituent group listed in Table 2 or Table 3, and combinations thereof;
wherein:
R₈ is selected from C₁-C₄ substituted or unsubstituted branched or unbranched alkyl and —CH₂—NR₁₄R₁₅;
R₉, R₁₀, R₁₄, and R₁₅ are each independently selected from H and C₁-C₄ substituted or unsubstituted branched or unbranched alkyl;
R₁₁ is selected from substituted or unsubstituted aryl and substituted or unsubstituted heteroaryl;
R₁₂ is selected from H, —(C═O)—R₁₃, and —NH-dimethylglycyl;
R₁₃ is C₁-C₄ substituted or unsubstituted branched or unbranched alkyl;
provided that R₁ and R₂ cannot both be —OH if R₃ is H; and
stereoisomers and pharmaceutically acceptable salts thereof.

In some aspects, the presently disclosed subject matter provides a method for treating a disease, disorder, or condition associated with excess and/or aberrant glutamine utilization, the method comprising administering to a subject in need of treatment thereof, a therapeutically effective amount of a compound of Formula (I) or a pharmaceutical formulation thereof.

In certain aspects, the disease, disorder, or condition associated with excess and/or aberrant glutamine utilization is selected from an infection, cancer, an autoimmune disease, an inflammatory disease, and a neurodegenerative or neurological disease.

In particular aspects, the cancer is selected from a newly diagnosed cancer, a recurrent cancer, a refractory cancer, and combinations thereof.

In more particular aspects, the cancer is selected from: (i) a cancer of the central nervous system; (ii) a cancer that is associated with transplant and/or immunosuppression; (iii) a cancer that is refractory to chemotherapy; (iv) a cancer that is refractory to photodynamic therapy; (v) a cancer that is refractory to proton therapy; (vi) a cancer that is refractory to radiotherapy; and (vii) a cancer that is refractory to surgery.

In yet more particular aspects, the cancer is selected from celnasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von lippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, lung cancer, testicular cancer, Hodgkin's disease, non-Hodgkin's lymphoma, oral cancer, skin cancer, mesothelioma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

In certain aspects, the method further comprises preventing a relapse in a cancer subject in remission.

In further aspects, the method further comprises administering a therapeutically effective amount of a compound of Formula (I) in combination with an immunotherapy.

In certain aspects, the immunotherapy includes a checkpoint blockade therapy. In particular aspects, the method comprises administering a compound of Formula (I) in combination with one or more checkpoint inhibitors. In more particular aspects, the one or more checkpoint inhibitors are selected from anti-PD-1 Ab, anti-PD-L1 Ab, anti-CTLA-4 Ab, anti-TIGIT Ab, and combinations thereof.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1 shows single time-point pharmacokinetic screening in mice demonstrating DON exposure in EL4 tumors (@30 or 60 minutes) and plasma of azotomycin prodrugs P1-P19;

FIG. 2A and FIG. 2B show the pharmacokinetics of P3 in mice. FIG. 2A shows the full pk of DON exposure of P3 in plasma, tumor, and jejunum. FIG. 2B shows the full pk intact prodrug exposure of P3 in plasma, tumor, and jejunum;

FIG. 2C and FIG. 2D show the pharmacokinetics of P4 in mice. FIG. 2C shows the full pk of DON exposure of P4 in plasma, tumor, and jejunum. FIG. 2D shows the full pk intact prodrug exposure of P4 in plasma, tumor, and jejunum; and

FIG. 2E shows the pharmacokinetics of P10 in mice. FIG. 2E shows the full pk of DON exposure of P10 in plasma, tumor, and jejunum.

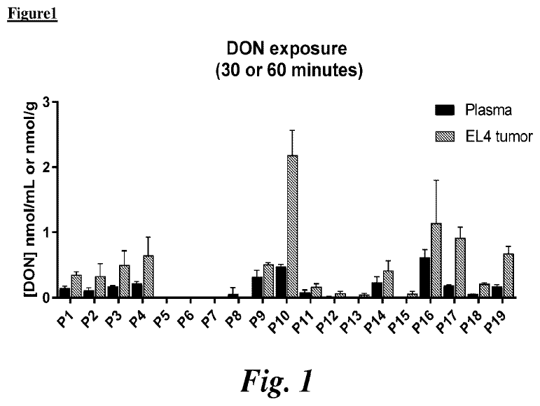

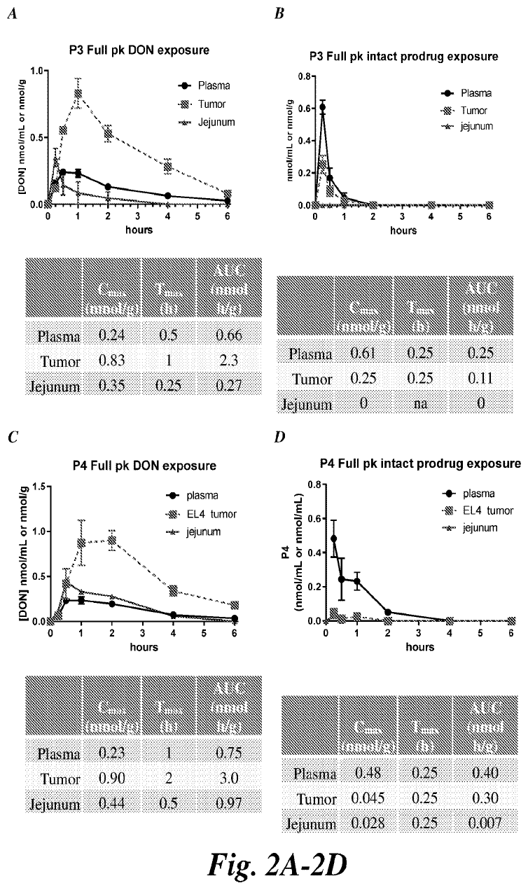

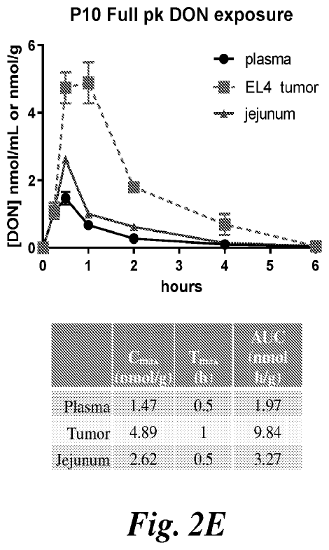

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Compounds of Formula (I)

In some embodiments, the presently disclosed subject matter provides a compound of formula (I):

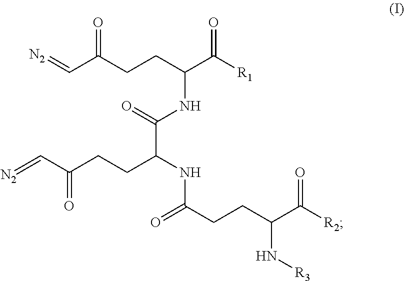

wherein:
R₁ and R₂ are each independently selected from —OR₄, —NR₅R₆, —O⁻M⁺, and —O—(CH₂CH₂O)ₙ—R₇, wherein:
n is an integer selected from 1, 2, 3, and 4;
R₄ is selected from H and C₁-C₄ substituted or unsubstituted branched or unbranched alkyl;
R₅ and R₆ are each independently H, C₁-C₄ substituted or unsubstituted branched or unbranched alkyl, -AA-COOR₇, wherein R₇ is C₁-C₄ substituted or unsubstituted branched or unbranched alkyl, and AA is an amino acid;
M⁺ is a metal cation, including, but not limited to Na⁺, K⁺, Li⁺, and, in some embodiments, NH₄⁺;
R₃ is selected from H, —(C═O)—R₈, —C(═O)—CH₂—(NH—C(═O)—CH₂—NR₉R₁₀)—CH₂—R₁₁, —C(═O)—CH—(NH—C(═O)—CH(NH₂)—CH—(CH₃)₂)—((CH₂)₃NH—C(═O)NH₂, —C(═O)—CH((CH₂)₄—NH—C(═O)—CH₃)(NH—C(═O)-adamantane), —C(═O)-AA-R₁₂, acetyl (Ac), dimethylglycine, dimethylglycyl (DMG), L-Lys(Ac), D-Val-L-Leu-L-Lys(Ac), D-Val-L-Leu-L-Lys(H), D-Val-L-Leu-L-Lys(DMG), L-Val-L-Cit, para-aminobenzoic acid (PABA), L-Cit-PABA, L-Val-L-Cit-PABA, Val-Cit-OH, dimethylglycyl-L-tryptophan, acetoxymethyl pivalate, 1-acetoxyethyl pivalate, acetoxy(phenyl)methyl pivalate, and 2-acetoxypropan-2-yl pivalate; wherein AA is an amino acid, wherein the amino acid of R₃, R₅, and R₆ can be selected from an amino acid or amino acid-related substituent group listed in Table 2 or Table 3, and combinations thereof;
wherein:
R₈ is selected from C₁-C₄ substituted or unsubstituted branched or unbranched alkyl and —CH₂—NR₁₄R₁₅;
R₉, R₁₀, R₁₄, and R₁₅ are each independently selected from H and C₁-C₄ substituted or unsubstituted branched or unbranched alkyl;
R₁₁ is selected from substituted or unsubstituted aryl and substituted or unsubstituted heteroaryl;
R₁₂ is selected from H, —(C═O)—R₁₃, and —NH-dimethylglycyl;
R₁₃ is C₁-C₄ substituted or unsubstituted branched or unbranched alkyl;
provided that R₁ and R₂ cannot both be —OH if R₃ is H; and
stereoisomers and pharmaceutically acceptable salts thereof.

One of ordinary skill in the art would recognize that azotomycin is explicitly excluded from the compounds of formula (I).

In certain embodiments, the compound of formula (I) is a compound of formula (Ia):

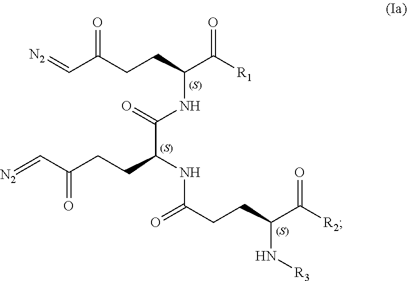

wherein:
R₁ and R₂ are each independently selected from —OR₄, —NR₅R₆, and —O—(CH₂CH₂O)ₙ—R₇, wherein:
n is an integer selected from 1, 2, 3, and 4;
R₄ is C₁-C₄ substituted or unsubstituted branched or unbranched alkyl;
R₅ and R₆ are each independently H, C₁-C₄ substituted or unsubstituted branched or unbranched alkyl, -AA-COOR₇, wherein R₇ is C₁-C₄ substituted or unsubstituted branched or unbranched alkyl, and AA is an amino acid;
R₃ is selected from H, —(C═O)—R₈, —C(═O)—CH(NR₉R₁₀)—R₇, —C(═O)—CH₂—(NH—C(═O)—CH₂—NR₉R₁₀)—CH₂—R₁₁, —C(═O)-AA-R₁₂, acetyl (Ac), dimethylglycine, dimethylglycyl (DMG), L-Lys(Ac), D-Val-L-Leu-L-Lys(Ac), D-Val-L-Leu-L-Lys(H), D-Val-L-Leu-L-Lys(DMG), L-Val-L-Cit, para-aminobenzoic acid (PABA), L-Cit-PABA, L-Val-L-Cit-PABA, dimethylglycyl-L-tryptophan, acetoxymethyl pivalate, 1-acetoxyethyl pivalate, acetoxy(phenyl)methyl pivalate, and 2-acetoxypropan-2-yl pivalate; wherein AA is an amino acid, wherein the amino acid of R₃, R₅, and R₆ can be selected from an amino acid or amino acid-related substituent group listed in Table 2 or Table 3, and combinations thereof;
wherein:
R₈ is selected from C₁-C₄ substituted or unsubstituted branched or unbranched alkyl and —CH₂—NR₁₄R₁₅;
R₉, R₁₀, R₁₄, and R₁₅ are each independently selected from H and C₁-C₄ substituted or unsubstituted branched or unbranched alkyl;
R₁₁ is selected from substituted or unsubstituted aryl and substituted or unsubstituted heteroaryl;
R₁₂ is selected from H, —(C═O)—R₁₃, and —NH-dimethylglycyl;
R₁₃ is C₁-C₄ substituted or unsubstituted branched or unbranched alkyl; and stereoisomers and pharmaceutically acceptable salts thereof.

In some embodiments, R₁ and R₂ are each independently selected from —OCH₂CH₃, —O—CH(CH₃)₂, —O—C(CH₃)₃, —NH₂, —NHCH₃, —NH-AA-COO—CH(CH₃)₂, —NH-AA-COO—C(CH₃)₃, —O—(CH₂CH₂O)˜-H, —O—(CH₂CH₂O)˜-AA-COO—CH(CH₃)₂, and —O—(CH₂CH₂O)˜-AA-COO—C(CH₃)₃.

In some embodiments, R₃ is selected from H, acetyl (Ac), dimethylglycine, dimethylglycyl (DMG), L-Lys(Ac), D-Val-L-Leu-L-Lys(Ac), D-Val-L-Leu-L-Lys(H), D-Val-L-Leu-L-Lys(DMG), L-Val-L-Cit, para-aminobenzoic acid (PABA), L-Cit-PABA, L-Val-L-Cit-PABA, dimethylglycyl-L-tryptophan, acetoxymethyl pivalate, 1-acetoxyethyl pivalate, acetoxy(phenyl)methyl pivalate, and 2-acetoxypropan-2-yl pivalate, —C(═O)-AA-H, —C(═O)-AA-Ac, and —C(═O)-AA-NH-dimethylglycyl.

In some embodiments, R₁ and R₂ are each —OR₄. In certain embodiments, one or both of R₁ and R₂ are each selected from —OCH₂CH₃, —OCH(CH₃)₂, and —OC(CH₃)₃.

In some embodiments, R₁ and R₂ are each —NR₅R₆. In certain embodiments, R₁ and R₂ are each —NH₂.

In some embodiments, R₃ is selected from H, —C(═O)—R₈, and —C(═O)—CH₂—(NH—C(═O)—CH₂—NR₉R₁₀)—CH₂—R₁. In some embodiments, R₃ is selected from H, —C(═O)—CH₃, —C(═O)—CH₂—N(CH₃)₂, and —C(═O)—CH₂—(NH—C(═O)—CH₂—N(CH₃)₂)—CH₂-(1H)indole.

In particular embodiments, the compound is selected from:

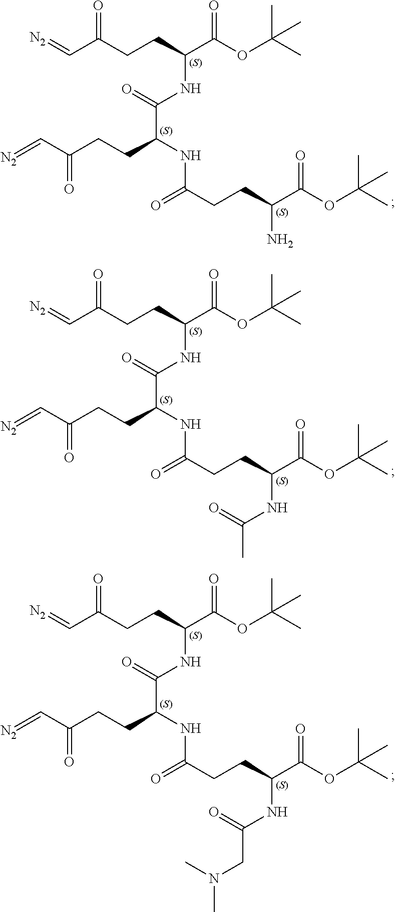

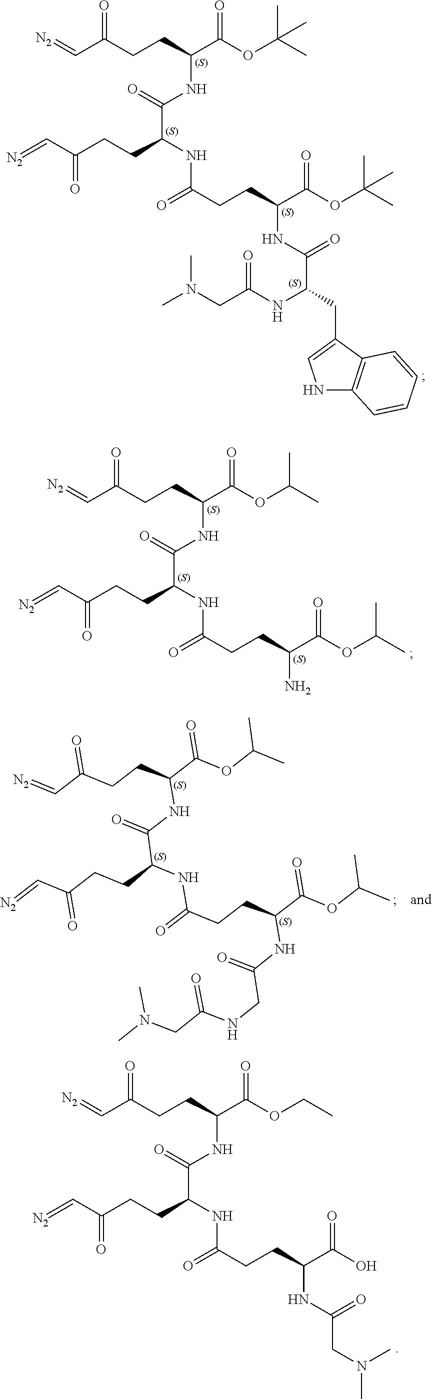

In more particular embodiments, the compound of formula (I) is selected from:

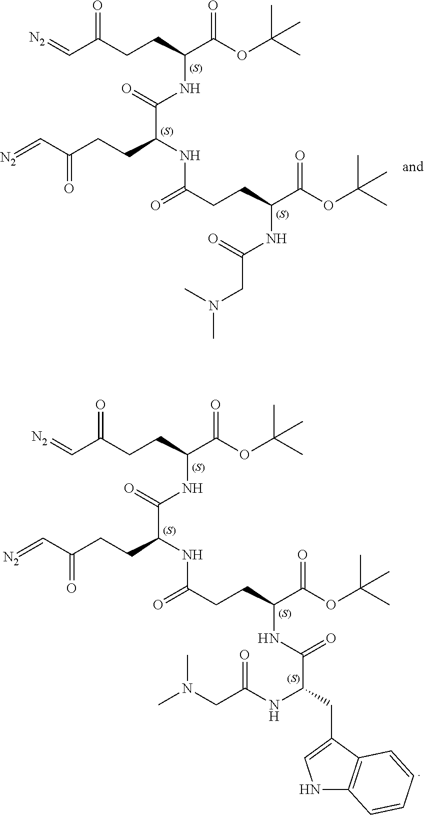

In even more particular embodiments, the compound of formula (I) is:

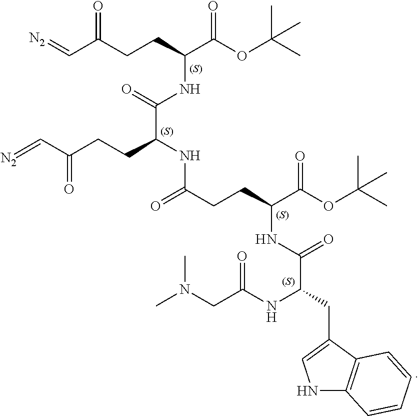

In particular embodiments, the compound is selected from:

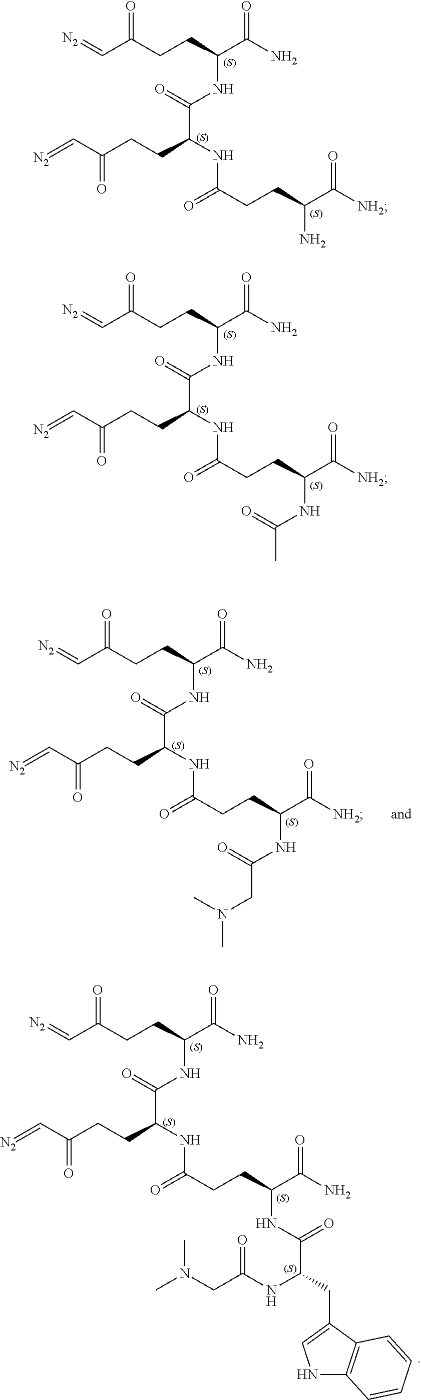

In certain embodiments, at least one of R₁ and R₂ is-O⁻M⁺. In particular embodiments, M⁺ is Na⁺ and the compound is selected from:

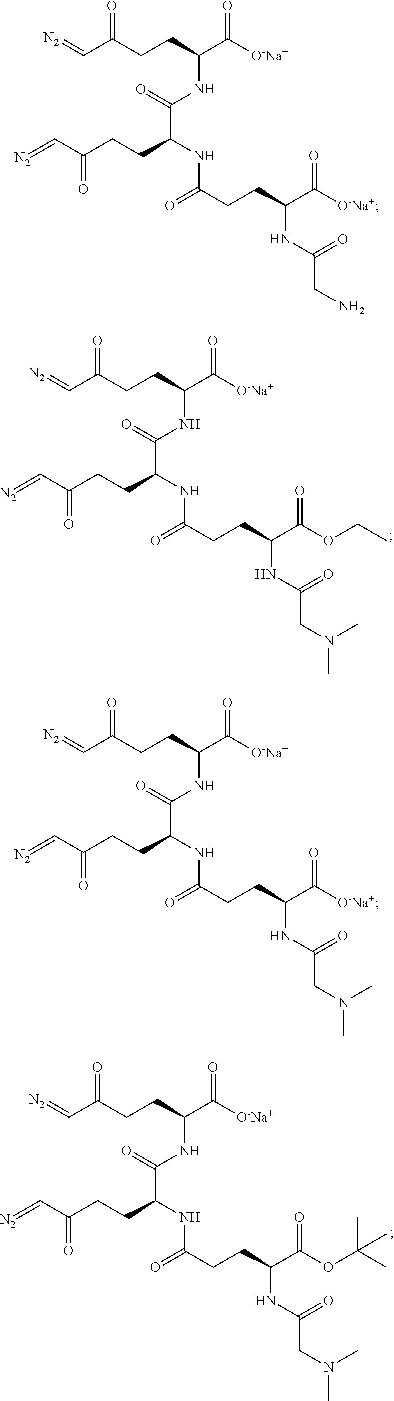

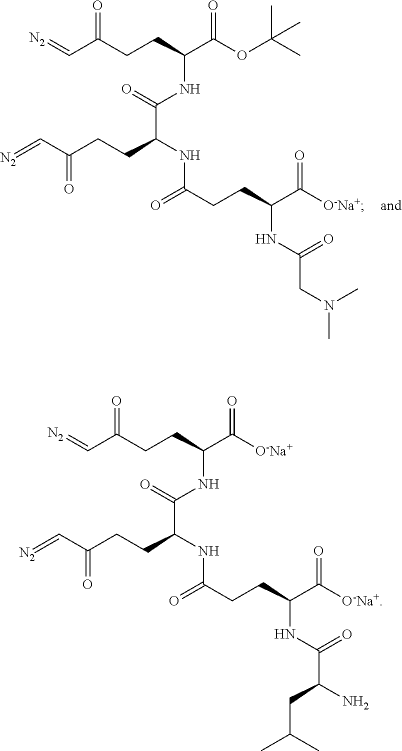

In certain embodiments, the compound is selected from:

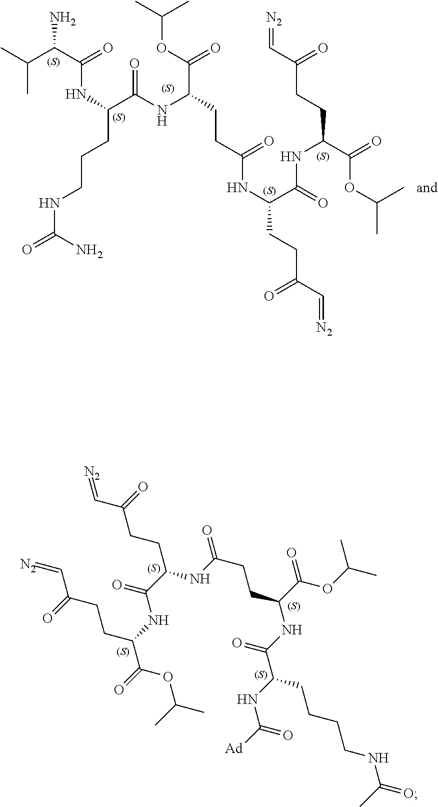

wherein Ad is adamantane.

Representative azotomycin prodrugs of Formula (I) are provided in Table 1.

TABLE 1
Representative azotomycin prodrugs of Formula (I)

| Prodrug | Structure | Molecular weight |
|---|---|---|
| | 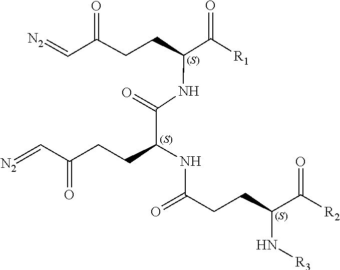 | |
| P1 | 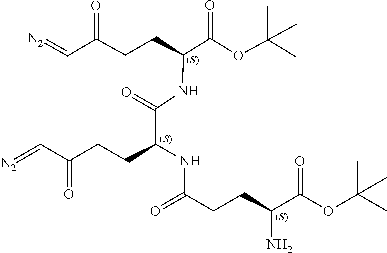 | 565.63 |
| P2 | 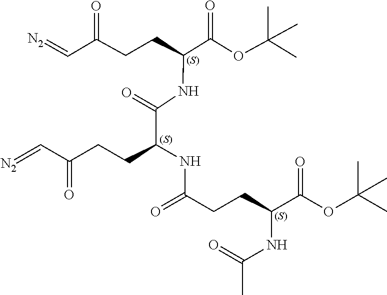 | 607.67 |
| P3 | 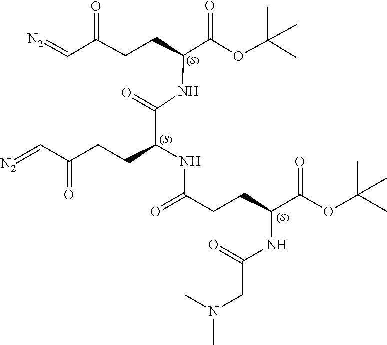 | 650.73 |
| P4 | 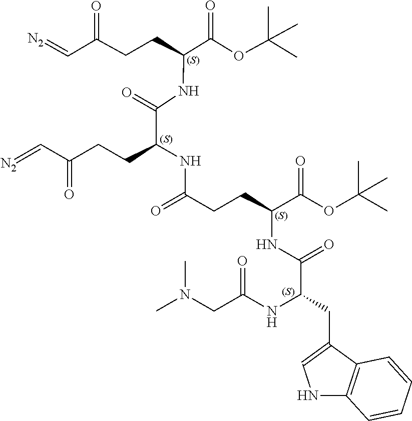 | 836.95 |
| P5 | 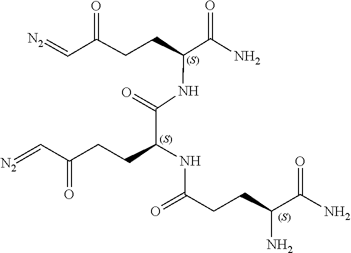 | 451.44 |
| P6 | 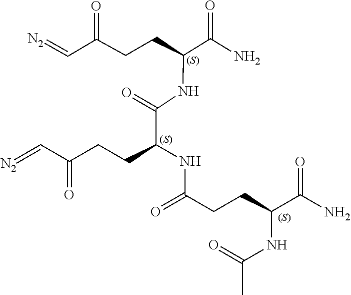 | 493.48 |
| P7 | 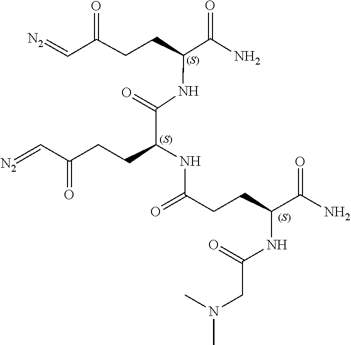 | 536.55 |
| P8 | 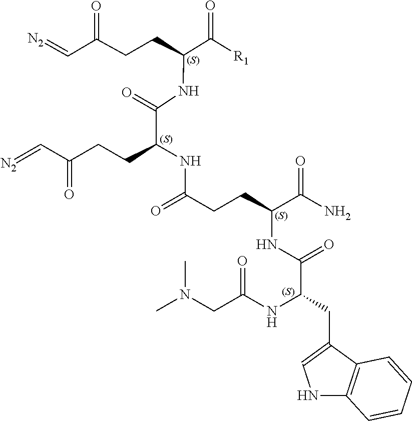 | 722.76 |
| P9 | 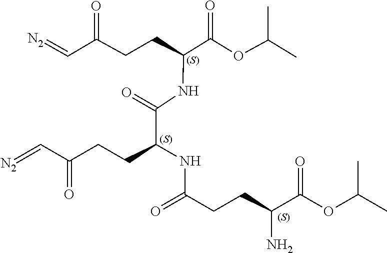 | 537.57 |
| P10 | 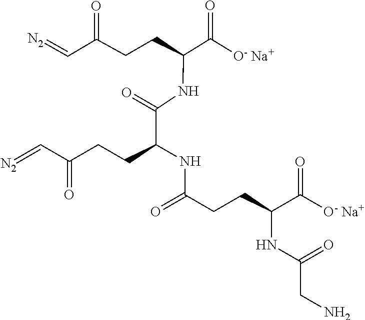 | 554.43 |
| P11 | 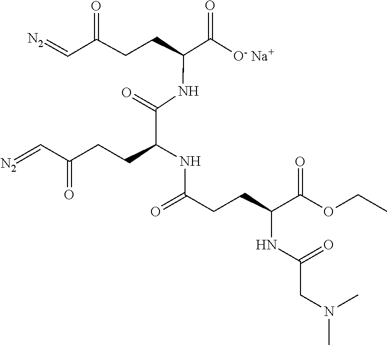 | 588.55 |
| P12 | 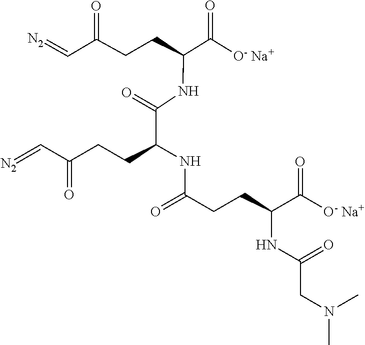 | 582.48 |
| P13 | 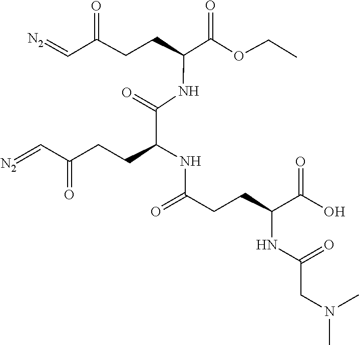 | 566.57 |
| P14 | 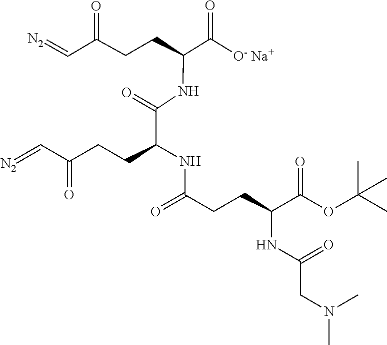 | 616.61 |
| P15 | 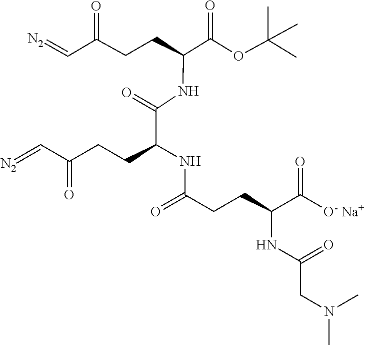 | 616.61 |
| P16 | 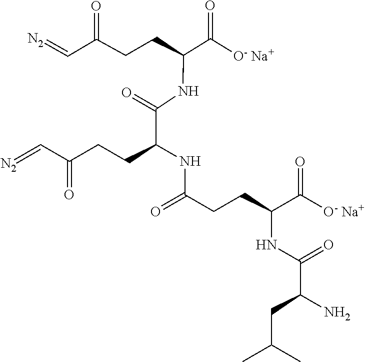 | 610.54 |
| P17 | 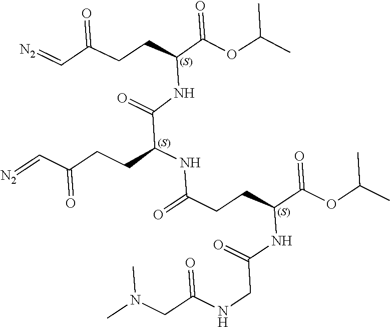 | 679.73 |
| P18 | 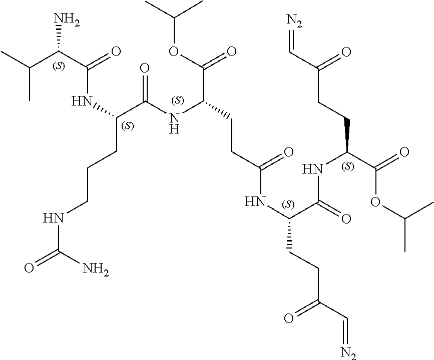 | 793.88 |
| P19 | 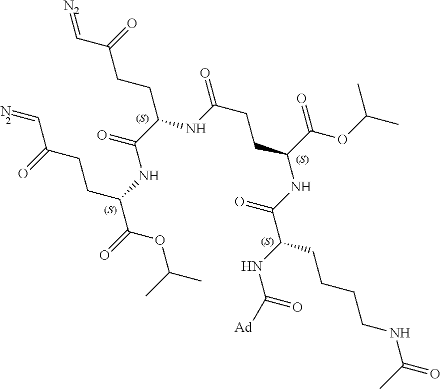 | 870.02 |

In some embodiments, the prodrug moiety can comprise an amino acid (AA). As used herein, the term “amino acid” includes moieties having a carboxylic acid group and an amino group. The term amino acid includes both natural amino acids (including proteinogenic amino acids) and non-natural amino acids. The term “natural amino acid” also includes other amino acids that can be incorporated into proteins during translation (including pyrrolysine and selenocysteine). Additionally, the term “natural amino acid” includes other amino acids that are formed during intermediary metabolism, e.g., ornithine generated from arginine in the urea cycle. A non-limiting list of representative amino acids is provided in Table 2. Representative amino acid-related substituent groups are provided in Table 3.

In some embodiments, the amino acid is selected from proteinogenic amino acids. Proteinogenic amino acids include glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine and histidine.

The term amino acid also includes alpha amino acids and beta amino acids, such as, but not limited to, beta alanine and 2-methyl beta alanine. The term amino acid also includes certain lactam analogues of natural amino acids, such as, but not limited to, pyroglutamine. The term amino acid also includes amino acid homologues including, but not limited to, homocitrulline, homoarginine, homoserine, homotyrosine, homoproline and homophenylalanine.

Examples of non-proteinogenic amino acids include, but are not limited to citrulline, hydroxyproline, 4-hydroxyproline, β-hydroxyvaline, ornithine, β-amino alanine, albizziin, 4-amino-phenylalanine, biphenylalanine, 4-nitro-phenylalanine, 4-fluoro-phenylalanine, 2,3,4,5,6-pentafluoro-phenylalanine, norleucine, cyclohexylalanine, α-aminoisobutyric acid, α-aminobutyric acid, α-aminoisobutyric acid, 2-aminoisobutyric acid, 2-aminoindane-2-carboxylic acid, selenomethionine, lanthionine, dehydroalanine, γ-amino butyric acid, naphthylalanine, aminohexanoic acid, pipecolic acid, 2,3-diaminoproprionic acid, tetrahydroisoquinoline-3-carboxylic acid, tert-leucine, tert-butylalanine, cyclopropylglycine, cyclohexylglycine, 4-aminopiperidine-4-carboxylic acid, diethylglycine, dipropylglycine and derivatives thereof wherein the amine nitrogen has been mono- or di-alkylated.

The terminal portion of the amino acid residue or peptide may be in the form of the free acid, i.e., terminating in a —COOH group, or may be in a masked (protected) form, such as in the form of a carboxylate ester or carboxamide. In certain embodiments, the amino acid or peptide residue terminates with an amino group. In an embodiment, the residue terminates with a carboxylic acid group —COOH or an amino group —NH₂. In another embodiment, the residue terminates with a carboxamide group. In yet another embodiment, the residue terminates with a carboxylate ester.

As disclosed hereinabove, the term “amino acid” includes compounds having a —COOH group and an —NH₂ group. A substituted amino acid includes an amino acid which has an amino group which is mono- or di-substituted. In particular embodiments, the amino group may be mono-substituted. (A proteinogenic amino acid may be substituted at another site from its amino group to form an amino acid which is a substituted proteinogenic amino acid). The term substituted amino acid thus includes N-substituted metabolites of the natural amino acids including, but not limited to, N-acetyl cysteine, N-acetyl serine, and N-acetyl threonine.

For example, the term “N-substituted amino acid” includes N-alkyl amino acids (e.g., C₁₋₆ N-alkyl amino acids, such as sarcosine, N-methyl-alanine, N-methyl-glutamic acid and N-tert-butylglycine), which can include C(N-substituted alkyl amino acids (e.g., N-(carboxy alkyl) amino acids (e.g., N-(carboxymethyl)amino acids) and N-methylcycloalkyl amino acids (e.g., N-methylcyclopropyl amino acids)); N,N-di-alkyl amino acids (e.g., N,N-di-C₁₋₆ alkyl amino acids (e.g., N,N-dimethyl amino acid)); N,N,N-tri-alkyl amino acids (e.g., N,N,N-tri-C₁₋₆ alkyl amino acids (e.g., N,N,N-trimethyl amino acid)); N-acyl amino acids (e.g., C₁₋₆ N-acyl amino acid); N-aryl amino acids (e.g., N-phenyl amino acids, such as N-phenylglycine); N-amidinyl amino acids (e.g., an N-amidine amino acid, i.e., an amino acid in which an amine group is replaced by a guanidino group).

The term “amino acid” also includes amino acid alkyl esters (e.g., amino acid C₁₋₆ alkyl esters); and amino acid aryl esters (e.g., amino acid phenyl esters). For amino acids having a hydroxy group present on the side chain, the term “amino acid” also includes O-alkyl amino acids (e.g., C₁₋₆, O-alkyl amino acid ethers); O-aryl amino acids (e.g., O-phenyl amino acid ethers); O-acyl amino acid esters; and O-carbamoyl amino acids. For amino acids having a thiol group present on the side chain, the term “amino acid” also includes S-alkyl amino acids (e.g., C₁₋₆ S-alkyl amino acids, such as S-methyl methionine, which can include C₁₋₆ S-substituted alkyl amino acids and S-methylcycloalkyl amino acids (e.g., S-methylcyclopropyl amino acids)); S-acyl amino acids (e.g., a C₁₋₆ S-acyl amino acid); S-aryl amino acid (e.g., a S-phenyl amino acid); a sulfoxide analogue of a sulfur-containing amino acid (e.g., methionine sulfoxide) or a sulfoxide analogue of an S-alkyl amino acid (e.g., S-methyl cystein sulfoxide) or an S-aryl amino acid.

The presently disclosed subject matter also envisages derivatives of natural amino acids, such as those mentioned above which have been functionalized by simple synthetic transformations known in the art (e.g., as described in “Protective Groups in Organic Synthesis” by T W Greene and P G M Wuts, John Wiley & Sons Inc. (1999)), and references therein.

In one embodiment, an amino acid side chain is bound to another amino acid. In a further embodiment, the side chain is bound to the amino acid via the amino acid's N-terminus. C-terminus, or side chain. Examples of natural amino acid sidechains include hydrogen (glycine), methyl (alanine), isopropyl (valine), sec-butyl (isoleucine), —CH₂CH(CH₃)₂(leucine), benzyl (phenylalanine), p-hydroxybenzyl (tyrosine), —CH₂OH (serine), —CH(OH)CH₃ (threonine), —CH₂-3-indoyl (tryptophan), —CH₂COOH (aspartic acid), —CH₂CH₂COOH (glutamic acid), —CH₂C(O)NH (asparagine), —CH₂CH₂C(O)NH₂ (glutamine), —CH₂SH, (cysteine), —CH₂CH₂SCH₃ (methionine), —(CH₂)₄NH₂ (lysine), —(CH₂)₃NHC(═NH)NH₂ (arginine) and —CH₂-3-imidazoyl (histidine).

In some embodiments, the amino acid can be substituted with a monocyclic ring. Exemplary monocyclic rings and bicyclic rings include, without limitation, benzene, pyrimidines, and purines, and more generally aryl and heteroaryl rings. Exemplary heteroaryls include, but are not limited to, pyridyl, pyrimidinyl, pyrazinyl, furanyl, thienyl, pyrazolyl, oxazolyl, thiazolyl, isothiazolyl, isoxazolyl, pyrrolyl, imidazolyl, indolyl, indolinolyl, and imidazopyridazinyl. Aryls include phenyl (C₆), benzyl, naphthyl (C₁₀), and biphenyl (C₁₂). Exemplary pyrimidines include, without limitation, cytosine, thymine, and uracil. Exemplary purines include, without limitation, purine, adenine, N-substituted adenine, guanine, hypoxanthine, xanthine, theobromine, caffeine, uric acid, and isoguanine. Exemplary purine nucleosides include, without limitation, adenine and guanine.

The term “peptide” refers to an amino acid chain consisting of 2 to 9 amino acids, unless otherwise specified. In preferred embodiments, the peptide used in the present invention is 2 or 3 amino acids in length. In one embodiment, a peptide can be a branched peptide. In this embodiment, at least one amino acid side chain in the peptide is bound to another amino acid (either through one of the termini or the side chain). The term “N-substituted peptide” refers to an amino acid chain consisting of 2 to 9 amino acids in which one or more NH groups are substituted, e.g., by a substituent described elsewhere herein in relation to substituted amino groups. Optionally, the N-substituted peptide has its N-terminal amino group substituted and, in one embodiment, the amide linkages are unsubstituted.

TABLE 2
Representative Amino Acids

| | Single | Multiple | |
|---|---|---|---|
| | Letter | Letter | |
| Name | Code | Code | Structure |
| Alanine | A | ALA | 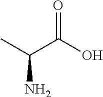 |
| Arginine | R | ARG | 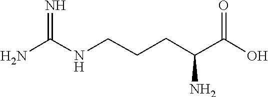 [SMILES: N=C(N)NCCC[C@H](N)C(=O)O] |
| Asparagine | N | ASN | 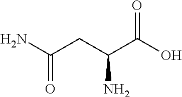 |
| Aspartic Acid | D | ASP | 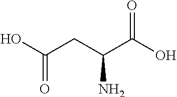 |
| Cysteine | C | CYS | 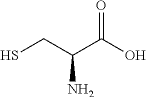 |
| Glutamic Acid | E | GLU | 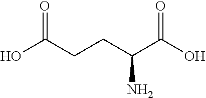 |
| Glutamine | Q | GLN | 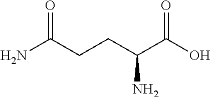 |
| Glycine | G | GLY | 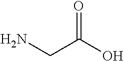 |
| Homocysteine | | HCY | 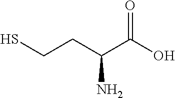 |
| Histidine | H | HIS | 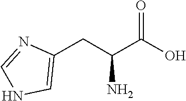 [SMILES: N[C@@H](Cc1c[nH]cn1)C(=O)O] |
| Homoserine | | HSE | 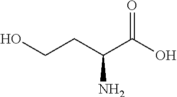 |
| Isoleucine | I | ILE | 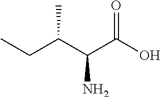 |
| Leucine | L | LEU | 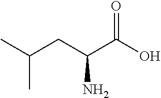 |
| Lysine | K | LYS | 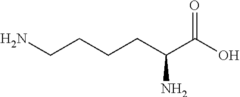 |
| Methionine | M | MET | 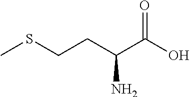 |
| Norleucine | | NLE | 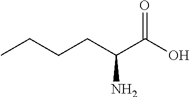 |
| Norvaline | | NVA | 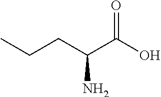 |
| Ornithine | | ORN | 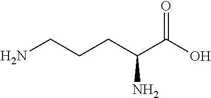 |
| Penicillamine | | PEN | 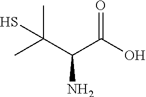 |
| Phenylalanine | F | PHE | 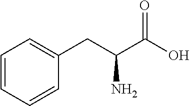 [SMILES: N[C@@H](Cc1ccccc1)C(=O)O] |
| Proline | P | PRO | 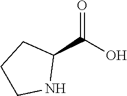 |
| Serine | S | SER | 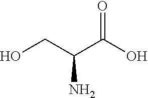 |
| Threonine | T | THR | 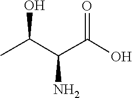 |
| Tryptophan | W | TRP | 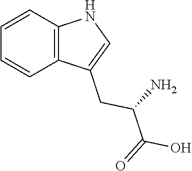 [SMILES: N[C@@H](Cc1c[nH]c2ccccc12)C(=O)O] |
| Tyrosine | Y | TYR | 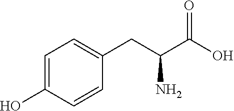 [SMILES: N[C@@H](Cc1ccc(O)cc1)C(=O)O] |
| Valine | V | VAL | 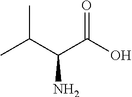 |
| Pyroglutamate | | pGLU | 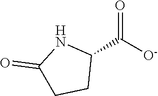 |
| Gamma-GLU | | gamma-GLU | 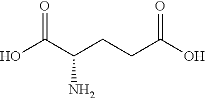 |
| Citrulline | | CIT | 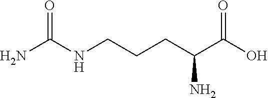 [SMILES: NC(=O)NCCC[C@H](N)C(=O)O] |
| Alpha Amino-Butyric Acid | | alpha-ABA | 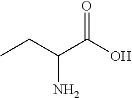 |
| D-Aspartic Acid | | | 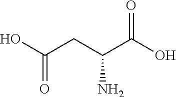 |
| 2-Methyl Alanine | | 2-Me-ALA | 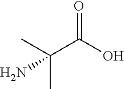 |
| Oxamic Acid | | OXA | 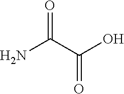 |
| Methionine sulfoxide | | Met(O) | 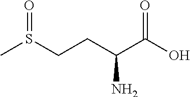 |
| Methionine sulfone | | Met(O)2 | 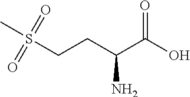 |
| Cyclopentylglycine | | Cpg | 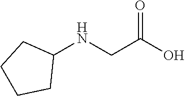 |
| Propargylglycine | | Pra | 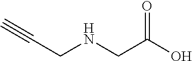 |
| 1,2,3,4- Tetrahydroisoquinoline- 3-carboxylic acid | | Tic | 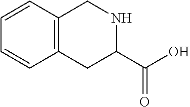 [SMILES: O=C(O)C1Cc2ccccc2CN1] |
| Selenocysteine | | Sec | 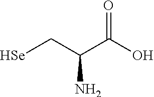 |
| Selenomethionine | | Se-Met | 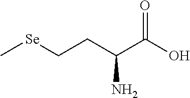 |
| Azido-Lysine | | Lys(N3) | 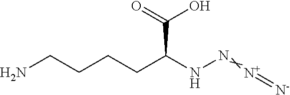 [SMILES: [N-]=[N+]=NN[C@@H](CCCCN)C(=O)O] |
| Beta-HomoLeucine | | Beta-HomoLeu | 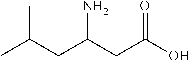 |
| Beta-Alanine | | Beta-Ala | 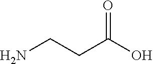 |
| 4-Aminobutyric acid | | GABA | 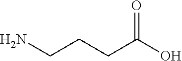 |
| 6-amino-hexanoic acid | | Ahx | 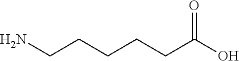 |
| 2,4-Diaminobutyric Acid | | Dab | 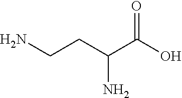 |
| 2,3-Diaminopropionic Acid | | Dap | 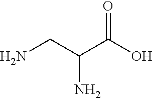 |
| alpha-aminoisobutyric acid (alpha-methyl- alanine) | | AIB | 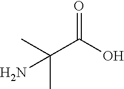 |
| L-alpha-aminobutyric acid | | ABU | 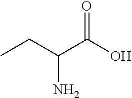 |
| Hydroxy Proline | | HYP | 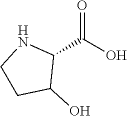 |
| beta-cyclohexyl-L-alanine | | Cha | 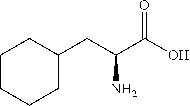 [SMILES: N[C@@H](CC1CCCCC1)C(=O)O] |
| Phenylglycine | | Phg | 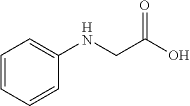 [SMILES: O=C(O)CNc1ccccc1] |

TABLE 3
Representative Amino Acid-Based Substituent Groups

| Name | Structure |
|---|---|
| Acetyl (Ac) | 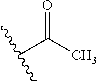 [SMILES: *C(C)=O] |
| Dimethylglycine | 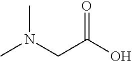 |
| Dimethylglycyl (DMG) | 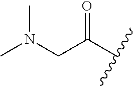 [SMILES: *C(=O)CN(C)C] |
| L-Lys(Ac) | 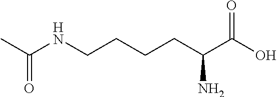 |
| D-Val-L-Leu-L-Lys(Ac) | 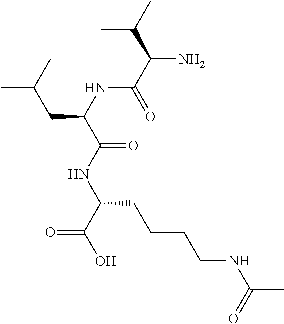 [SMILES: CC(=O)NCCCC[C@@H](NC(=O)[C@@H](CC(C)C)NC(=O)[C@H](N)C(C)C)C(=O)O] |
| D-Val-L-Leu-L-Lys(H) | 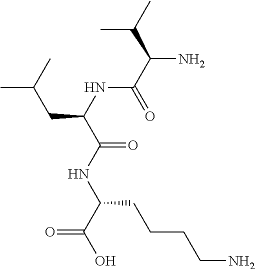 [SMILES: CC(C)C[C@@H](NC(=O)[C@H](N)C(C)C)C(=O)N[C@H](CCCCN)C(=O)O] |
| D-Val-L-Leu-L-Lys(DMG) | 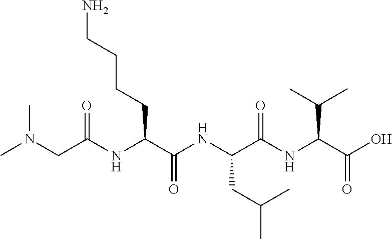 [SMILES: CC(C)C[C@H](NC(=O)[C@H](CCCCN)NC(=O)CN(C)C)C(=O)N[C@H](C(=O)O)C(C)C] |
| L-Val-L-Cit | 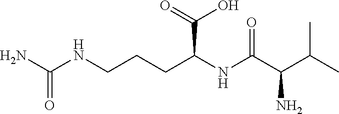 [SMILES: CC(C)[C@@H](N)C(=O)N[C@@H](CCCNC(N)=O)C(=O)O] |
| Para-aminobenzoic acid (PABA) | 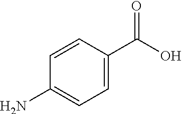 |
| L-Cit-PABA | 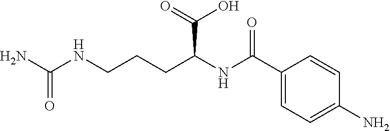 [SMILES: NC(=O)NCCC[C@H](NC(=O)c1ccc(N)cc1)C(=O)O] |
| L-Val-L-Cit-PABA | 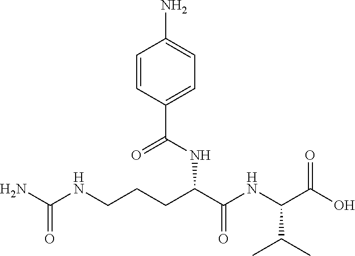 [SMILES: CC(C)[C@H](NC(=O)[C@H](CCCNC(N)=O)NC(=O)c1ccc(N)cc1)C(=O)O] |
| dimethylglycyl-L- tryptophan | 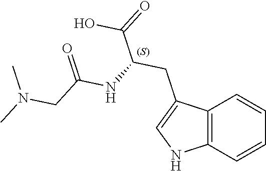 |
| acetoxymethyl pivalate | 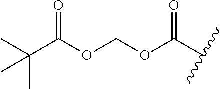 [SMILES: *C(=O)OCOC(=O)C(C)(C)C] |
| 1-acetoxyethyl pivalate | 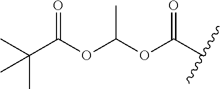 [SMILES: *C(=O)OC(C)OC(=O)C(C)(C)C] |
| acetoxy(phenyl)methyl pivalate | 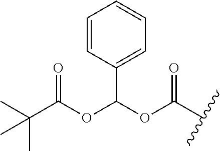 [SMILES: *C(=O)OC(OC(=O)C(C)(C)C)c1ccccc1] |
| 2-acetoxypropan-2-yl pivalate | 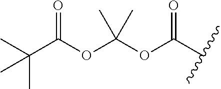 [SMILES: *C(=O)OC(C)(C)OC(=O)C(C)(C)C] |

### II. Methods of Treatment

In some embodiments, the presently disclosed subject matter provides a method for treating a disease, disorder, or condition associated with excess and/or aberrant glutamine utilization, the method comprising administering to a subject in need of treatment thereof, a therapeutically effective amount of a compound of Formula (I) or a pharmaceutical formulation thereof.

In certain embodiments, the disease, disorder, or condition associated with excess and/or aberrant glutamine utilization is selected from an infection, cancer, an autoimmune disease, an inflammatory disease, and a neurodegenerative or neurological disease.

In particular embodiments, the cancer is selected from a newly diagnosed cancer, a recurrent cancer, a refractory cancer, and combinations thereof.

In more particular embodiments, the cancer is selected from: (i) a cancer of the central nervous system; (ii) a cancer that is associated with transplant and/or immunosuppression; (iii) a cancer that is refractory to chemotherapy; (iv) a cancer that is refractory to photodynamic therapy; (v) a cancer that is refractory to proton therapy; (vi) a cancer that is refractory to radiotherapy; and (vii) a cancer that is refractory to surgery.

In yet more particular embodiments, the cancer is selected from celnasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, lung cancer, testicular cancer, Hodgkin's disease, non-Hodgkin's lymphoma, oral cancer, skin cancer, mesothelioma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

In certain embodiments, the method further comprises preventing a relapse in a cancer subject in remission.

In other embodiments, the presently disclosed method further comprises administering a therapeutically effective amount of a compound of Formula (I) in combination with an immunotherapy. In certain embodiments, the immunotherapy includes a checkpoint blockade therapy. In particular embodiments, the method comprises administering a compound of Formula (I) in combination with one or more checkpoint inhibitors. In more particular embodiments, the one or more checkpoint inhibitors are selected from anti-PD-1 Ab, anti-PD-Li Ab, anti-CTLA-4 Ab, anti-TIGIT Ab, and combinations thereof. See, for example, Yokoyama, Y. et al., 2022; Rais, R. et al., 2022; and Leone R. D., et al., 2019.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compositions can be administered prophylactically to prevent or reduce the incidence or recurrence of the disease, disorder, or condition.

As used herein, the term “inhibit” or “inhibits” means to decrease, suppress, attenuate, diminish, arrest, or stabilize an activity of an agent, e.g., an enzyme, associated with a disease or a disease-related pathway or the development or progression of a disease, disorder, or condition, e.g., a brain cancer, by at least 10%, 20%, 30%, 40%, 50%, 60%, 70%, 80%, 90%, 95%, 98%, 99%, or even 100% compared to an untreated control subject, cell, biological pathway, or biological activity.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent refers to the amount necessary to elicit the desired biological response. As will be appreciated by those of ordinary skill in this art, the effective amount of an agent may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a compound of formula (I) described herein and at least one other therapeutic agent, such as a chemotherapeutic agent or an immunotherapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compositions described herein can be administered alone or in combination with adjuvants that enhance stability of the compositions alone or in combination with one or more therapeutic agents, facilitate administration of pharmaceutical compositions containing them in certain embodiments, provide increased dissolution or n dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound of Formula (I) described herein and at least one additional therapeutic agent can be varied so long as the beneficial effects of the combination of these agents are achieved. Accordingly, the phrase “in combination with” refers to the administration of a compound of Formula (I) described herein and at least one additional therapeutic agent either simultaneously, sequentially, or a combination thereof. Therefore, a subject administered a combination of a compound of Formula (I) described herein and at least one additional therapeutic agent can receive a and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the described herein and at least one additional therapeutic agent are administered simultaneously, they can be administered to the subject as separate pharmaceutical compositions, each comprising either a compound of Formula (I) or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of an miR-described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( S I )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### III. Pharmaceutical Compositions and Administration

In some embodiments, the present disclosure provides a pharmaceutical composition including one presently disclosed compound of Formula (I) alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, trifluoroacetic acid (TFA), and the like. Also included are salts of amino acids such as arginate and the like, and salts of organic acids like glucuronic or galacturonic acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions, and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

In particular embodiments, the presently disclosed compound of Formula (I) is administered intranasally in a form selected from the group consisting of a nasal spray, a nasal drop, a powder, a granule, a cachet, a tablet, an aerosol, a paste, a cream, a gel, an ointment, a salve, a foam, a paste, a lotion, a cream, an oil suspension, an emulsion, a solution, a patch, and a stick. As used herein, the term administrating via an “intranasal route” refers to administering by way of the nasal structures.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### IV. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of formulae (I-III) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a univalent group derived from an alkane by removal of a hydrogen atom from any carbon atom —CₙH₂ₙ₊₁. The groups derived by removal of a hydrogen atom from a terminal carbon atom of unbranched alkanes form a subclass of normal alkyl (n-alkyl) groups H(CH₂)ₙ. The groups RCH₂, R₂CH (R≠H), and R₃C (R≠H) are primary, secondary and tertiary alkyl groups, respectively. An alkyl can be a straightchain (i.e., unbranched) or branched acyclic hydrocarbon having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons.

Representative alkyl groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, and dodecyl. Representative C₁-C₄ alkyl groups include methyl, ethyl, n-propyl, isopropyl, n-butyl, isobutyl, sec-butyl, and tert-butyl.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CHO—CH₃, —Si(CH₃)₃, —CH₂—CH═N—OCH₃, —CH═CH—N(CH₃)— CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a univalent group derived from a cycloalkane by removal of a hydrogen atom from a ring carbon atom. A cycloalkane is a saturated monocyclic hydrocarbon (with or without side chains), e.g., cyclobutane. Unsaturated monocyclic hydrocarbons having one endocyclic double or one triple bond are called cycloalkenes and cycloalkynes, respectively. Those having more than one such multiple bond are cycloalkadienes, cycloalkatrienes, and the like. The inclusive terms for any cyclic hydrocarbons having any number of such multiple bonds are cyclic olefins or cyclic acetylenes. As used herein, cycloalkyls can be a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopropyl, cyclobutyl, cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

As used herein the terms “bicycloalkyl” and “bicycloheteroalkyl” refer to two cycloalkyl or cycloheteroalkyl groups that are bound to one another. Non-limiting examples include bicyclohexane and bipiperidine.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, a substituent group derived from an arene, i.e., a monoyclic or polycyclic aromatic hydrocarbon, by removal of a hydrogen atom from a ring carbon atom. An aryl group can include an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

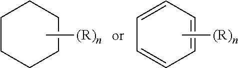

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

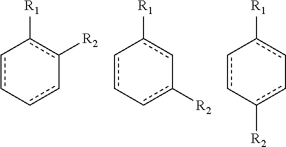

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN, CF₃, fluorinated C₁₋₄ alkyl, and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁₋₄)alkoxo, and fluoro(C₁₋₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3.

Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)₅—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group.

Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂.

“Alkylcarbamoyl” refers to a R′RN—C(═O)— group wherein one of R and R′ is hydrogen and the other of R and R′ is alkyl and/or substituted alkyl as previously described.

“Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom.

The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The term “cyano” refers to the —C≡N group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms.

For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, 100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

The synthetic descriptions and specific examples that follow are only intended for the purposes of illustration, and are not to be construed as limiting in any manner to make compositions of the disclosure by other methods.

### Example 1

Azotomycin Prodrugs with Preferential Tumor Delivery

### 1.1 Overview

The presently disclosed subject matter provides the development of novel tumor cell-targeted azotomycin prodrugs intended to circulate intact as inert prodrug/s in plasma and be preferentially biotransformed to its active glutamine antagonist metabolite “DON” in tumor cells. Several azotomycin prodrugs were synthesized and subsequently characterized using a well-defined screening paradigm. Through this screening process, two prodrugs, P3 and P4, that exhibited preferential tumor delivery were identified. P3 and P4 were both stable in mouse and human plasma and liver microsomes in vitro.

When administered subcutaneously in C57BL/6 CES1^{-/-} mice bearing flank EL4 tumors P3 exhibited excellent pharmacokinetics with an approximately 3.5-fold higher DON tumor exposures versus plasma.

Most importantly, P3 exhibited an approximately 8-fold higher tumor exposures (efficacy site; AUC=2.3 nmol/mL*h) versus GI-tissues (toxicity site; AUC=0.27 nmol/mL*h). P4 also showed tumor targeting in mice although its preferential delivery was not as significant versus P3. P3 showed a 3-fold enhanced exposure in tumor (efficacy site; AUC=3.0 nmol/mL*h) versus GI-tissues (toxicity site; AUC=0.97 nmol/mL*h). In sum, the presently disclosed subject matter demonstrates the rationale design and discovery of tumor-targeted azotomycin glutamine antagonist prodrugs with significantly less GI exposure versus tumor. Given GI toxicity had previously hampered the development of this efficacious class of therapeutics, it is thought that these prodrugs could be developed for future clinical studies.

### 1.2 Experimental

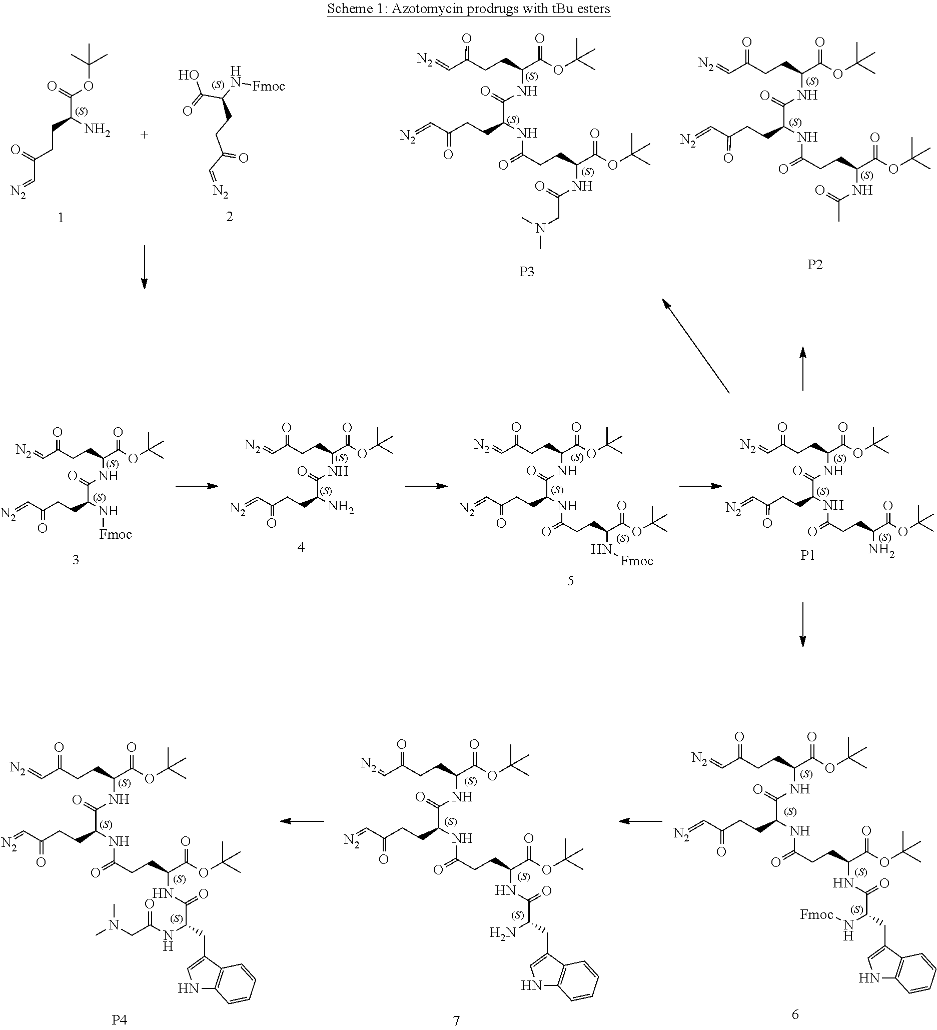

### tert-Butyl (S)-2-((S)-2-((((9H-fluoren-9-yl)methoxy)carbonyl)amino)-6-diazo-5-oxohexanamido)-6-diazo-5-oxohexanoate (3)

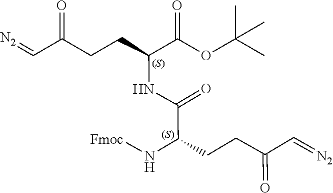

Fmoc-L-DON-OH (1.65 g, 4.18 mmol, 1 equiv) and HATU (1.67 g, 4.39 mmol, 1.05 equiv) were dissolved in anhydrous DCM (40 mL), the mixture was cooled to 0° C. and DIPEA (1.62 g, 2.18 mL, 12.5 mmol, 3 equiv) was added. The mixture was stirred for 5 minutes and solution of H-L-DON-OtBu (950 mg, 4.18 mmol, 1 equiv) in anhydrous DCM (12 mL) was added. The resulting mixture was stirred for 30 minutes at 0° C. and 1.5 h at room temperature. DCM was evaporated, the residue was dissolved in EtOAc (400 mL) and washed with sat. NaHCO₃ (200 mL), 10% KHSO₄ (200 mL), H₂O (200 mL) and brine (200 ml), dried over MgSO₄ and DCM was evaporated. The crude product was purified by LC on silica (DCM/EtOAc, 1:1) and product 3 was obtained as a yellow solid (1.60 g) in 63% yield.

¹H NMR (CDCl₃): 1.48 (s, 9H), 1.96-2.07 (m, 2H), 2.14-2.28 (m, 2H), 2.31-2.46 (m, 2H), 2.48-2.65 (m, 2H), 4.24 (q, J=7.6, 7.1 Hz, 2H), 4.38 (d, J=7.3 Hz, 2H), 4.45 (td, J=8.1, 4.6 Hz, 1H), 5.26 (bs, 1H), 5.35 (bs, 1H), 5.95 (d, J=7.5 Hz, 1H), 7.27-7.31 (m, 1H), 7.33 (tt, J=7.4, 1.0 Hz, 2H), 7.36-7.45 (m, 2H), 7.60 (dd, J=7.8, 3.0 Hz, 2H), 7.75 (dq, J=7.6, 1.0 Hz, 2H). ESI-MS: 625.400 ([M+Na]+).

### tert-Butyl (S)-2-((S)-2-amino-6-diazo-5-oxohexanamido)-6-diazo-5-oxohexanoate (4)

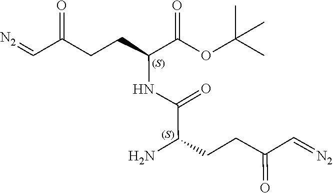

Compound 3 (1.29 g, 2.14 mmol, 1 equiv) was dissolved in anhydrous DCM (15 mL), diethylamine (2.20 mL, 21.4 mmol, 10 equiv) was added and the mixture was stirred at room temperature under nitrogen atmosphere for 2.5 h. Volatiles were removed under vacuo and the crude product 4 was immediately used to the following step without purification.

### tert-Butyl (5S,10S,13S)-5-(tert-butoxycarbonyl)-10,13-bis(4-diazo-3-oxobutyl)-1-(9H-fluoren-9-yl)-3,8,11-trioxo-2-oxa-4,9,12-triazatetradecan-14-oate (5)

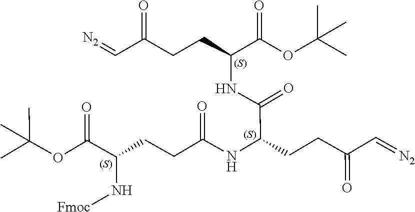

Fmoc-L-Glu(H)-OtBu (765 mg, 1.80 mmol, 1 equiv) and HATU (752 mg, 1.98 mmol, 1.1 equiv) were dissolved in anhydrous DCM (15 mL), the mixture was cooled to 0° C. and DIPEA (0.94 mL, 5.39 mmol, 3.0 equiv) was added. After 5 minutes solution of compound 4 (684 mg, 1.80 mmol, 1.0 equiv) in anhydrous DCM (5 mL) was added and the mixture was stirred for 30 minutes at 0° C. and overnight (16 h) at room temperature. DCM was evaporated and the residue was dissolved in EtOAc (250 mL) and washed with sat. NaHCO₃ (100 mL), H₂O (100 mL), 10% KHSO₄ (100 mL) and brine (100 mL). The organic layer was dried over MgSO₄ and EtOAc was evaporated. The crude product was purified by LC on silica (DCM/MeOH, 30:1) and product 5 was obtained as a yellow solid (734 mg, 54% yield over 2 steps).

¹H NMR (CDCl₃): 1.45 (s, 9H), 1.47 (s, 9H), 1.87-2.01 (m, 3H), 2.11-2.26 (m, 3H), 2.28-2.42 (m, 4H), 2.46-2.66 (m, 2H), 4.20-4.27 (m, 2H), 4.35-4.45 (m, 4H), 5.30 (bs, 1H), 5.34 (bs, 1H), 5.64 (d, J=8.1 Hz, 1H), 6.81 (d, J=7.0 Hz, 1H), 7.24 (m, 1H), 7.28-7.45 (m, 4H), 7.61 (d, J=7.4 Hz, 2H), 7.73-7.80 (m, 2H). ESI-MS: 810.647 ([M+Na]⁺).

### tert-Butyl (S)-2-((S)-2-((S)-4-amino-5-(tert-butoxy)-5-oxopentanamido)-6-diazo-5-oxohexanamido)-6-diazo-5-oxohexanoate (P1)

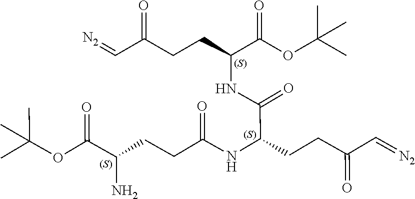

Compound 5 (734 mg, 0.932 mmol, 1 equiv) was dissolved in anhydrous DCM (7.5 mL), diethylamine (1.93 mL, 18.6 mmol, 20 equiv) was added and the mixture was stirred at room temperature under nitrogen atmosphere for 2 h. Volatiles were removed under vacuo and the residue was purified by LC on silica (DCM/MeOH, 15:1). Product P1 was isolated as a yellow solid (350 mg) in 66% yield.

¹H NMR (CDCl₃): 1.45 (s, 9H), 1.46 (s, 9H), 1.74-1.90 (m, 3H), 1.93-2.09 (m, 3H), 2.09-2.25 (m, 2H), 2.29-2.45 (m, 4H), 2.44-2.65 (m, 2H), 3.34 (dd, J=8.7, 4.8 Hz, 1H), 4.36-4.46 (m, 2H), 5.36 (2×CH, bs, 2H), 7.04 (d, J=7.1 Hz, 1H), 7.42 (d, J=7.9 Hz, 1H). ESI-MS: 566.416 ([M+H]⁺).

### tert-Butyl (S)-2-((S)-2-((S)-4-acetamido-5-(tert-butoxy)-5-oxopentanamido)-6-diazo-oxohexanamido)-6-diazo-5-oxohexanoate (P2)

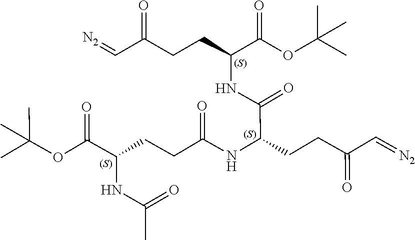

Compound P1 (200 g, 0.354 mmol, 1 equiv) was dissolved in anhydrous DCM (2 mL) and pyridine (57 μL, 0.707 mmol, 2.0 equiv) followed by acetanhydride (40 μL, 0.424 mmol, 1.2 equiv) were added. The reaction mixture was stirred for 1 h at room temperature under inert nitrogen atmosphere. Volatiles were removed under vacuo and the residue was purified by LC on silica (DCM/MeOH, 30:1 to 10:1). Product P2 was isolated as a yellow solid (143 mg) in 67% yield.

¹H NMR (CDCl₃): 1.46 (s, 9H), 1.47 (s, 9H), 1.90 (ddd, J=14.1, 7.1, 2.1 Hz, 1H), 1.94-2.01 (m, 2H), 2.02 (s, 3H), 2.10-2.26 (m, 3H), 2.28-2.34 (m, 2H), 2.33-2.46 (m, 2H), 2.47-2.65 (m, 2H), 4.36-4.50 (m, 3H), 5.36 (bs, 1H), 5.38 (bs, 1H), 6.43 (d, J=7.7 Hz, 1H), 7.01 (d, J=7.0 Hz, 1H), 7.26-7.29 (m, 1H). ESI-MS: 630.490 ([M+Na]⁺).

### tert-Butyl (6S,11S,14S)-6-(tert-butoxycarbonyl)-11,14-bis(4-diazo-3-oxobutyl)-2-methyl-4,9,12-trioxo-2,5,10,13-tetraazapentadecan-15-oate (P3)

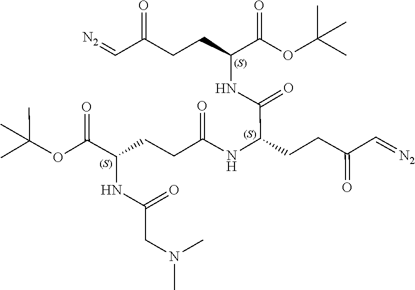

Compound P1 (158 mg, 0.279 mmol, 1 equiv) was dissolved in anhydrous DCM (6 mL) and 2,5-dioxopyrrolidin-1-yl dimethylglycinate (67 mg, 0.335 nmol, 1.2 equiv) was added and the mixture was stirred at room temperature under nitrogen atmosphere for 3 h. DCM (50 mL) was added and the organic phase was washed with sat. NaHCO₃ (2×30 mL) and brine (30 mL), dried over anhydrous MgSO₄ and solvent was evaporated. Crude product was purified by LC on silica (DCM/MeOH, 10:1) and product P3 was isolated as a yellow solid (178 mg, 98%).

¹H NMR (CDCl₃): 1.45 (s, 9H), 1.47 (s, 9H), 1.65-1.81 (m, 1H), 1.82-1.93 (m, 1H), 1.93-2.05 (m, 2H), 2.11-2.25 (m, 4H), 2.33 (s, 6H), 2.35-2.70 (m, 4H), 2.92 (d, J=16.3 Hz, 1H), 3.04 (d, J=16.3 Hz, 1H), 4.37-4.45 (m, 2H), 4.49 (td, J=9.1, 3.9 Hz, 1H), 5.36 (bs, 1H), 5.39 (bs, 1H), 7.10 (d, J=6.6 Hz, 1H), 7.24 (d, J=8.0 Hz, 1H), 7.71 (d, J=8.7 Hz, 1H). ESI-MS: 651.513 ([M+H]⁺).

### tert-Butyl (5S,8S,13S,16S)-5-((1H-indol-3-yl)methyl)-8-(tert-butoxycarbonyl)-13,16-bis(4-diazo-3-oxobutyl)-1-(9H-fluoren-9-yl)-3,6,11,14-tetraoxo-2-oxa-4,7,12,15-17-oate (6)

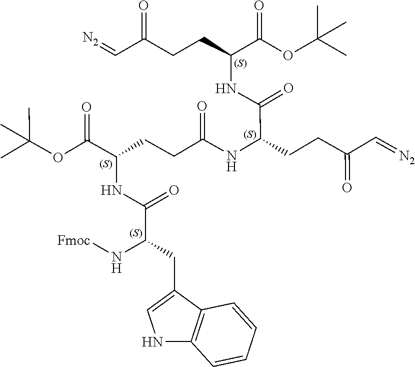

Fmoc-L-Trp-OH (249 mg, 0.583 mmol, 1.1 equiv) and HATU (232 mg, 0.610 mmol, 1.15 equiv) were dissolved in anhydrous DMF (10 mL), the mixture was cooled to 0° C. and DIPEA (277 μL, 1.59 mmol, 3.0 equiv) was added. After 5 minutes solution of compound P1 (300 mg, 0.530 mmol, 1.0 equiv) in anhydrous DMF (6 mL) was added and the mixture was stirred for 30 minutes at 0° C. and 2 h at room temperature. DMF was evaporated and the residue was purified by reverse phase HPLC (AcN/H₂O) and product 6 was obtained as a yellow solid (448 mg, 88% yield).

¹H NMR (CDCl₃): 1.41 (s, 9H), 1.46 (s, 9H), 1.86-2.07 (m, 4H), 2.10-2.28 (m, 4H), 2.30-2.63 (m, 4H), 3.11-3.22 (m, 1H), 3.47 (dd, J=14.9, 4.8 Hz, 1H), 4.22 (t, J=7.1 Hz, 1H), 4.26-4.50 (m, 5H), 4.60-4.71 (m, 1H), 5.30 (bs, 1H), 5.53 (bs, 1H), 6.81-6.93 (m, 2H), 7.11-7.24 (m, 4H), 7.28-7.46 (m, 6H), 7.54-7.60 (m, 2H), 7.60-7.67 (m, 1H), 7.77 (d, J=7.6 Hz, 2H), 9.00-9.10 (m, 1H). ESI-MS: 996.725 ([M+Na]⁺).

### tert-Butyl (S)-2-((S)-2-((S)-4-((S)-2-amino-3-(1H-indol-3-yl)propanamido)-5-(tert-butoxy)-5-oxopentanamido)-6-diazo-5-oxohexanamido)-6-diazo-5-oxohexanoate (7)

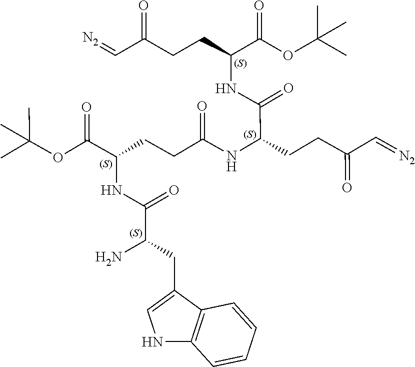

Compound 6 (445 mg, 0.457 mmol, 1 equiv) was dissolved in anhydrous DCM (5 mL), diethylamine (946 μL, 9.14 mmol, equiv) was added and the mixture was stirred at room temperature under nitrogen atmosphere for 2 h. Volatiles were removed under vacuo and the residue was purified by LC on silica (DCM/MeOH, 15:1 to 10:1). Product 7 was isolated as a yellow solid (270 mg) in 79% yield.

¹H NMR (CDCl₃): 1.44 (s, 9H), 1.47 (s, 9H), 1.57-1.78 (m, 2H), 1.81-2.07 (m, 4H), 2.08-2.30 (m, 4H), 2.56 (d, J=17.7 Hz, 4H), 3.26 (dd, J=14.5, 5.3 Hz, 2H), 3.80 (dd, J=6.1, 4.5 Hz, 1H), 4.33-4.50 (m, 3H), 5.33 (bs, 1H), 5.36 (bs, 1H), 6.83 (d, J=7.0 Hz, 1H), 7.07-7.22 (m, 3H), 7.32 (d, J=7.6 Hz, 1H), 7.39 (dt, J=8.2, 1.0 Hz, 1H), 7.63-7.70 (m, 1H), 7.92 (d, J=8.5 Hz, 1H), 9.08 (s, 1H). ESI-MS: 752.312 ([M+H]⁺).

### tert-Butyl (6S,9S,14S,17S)-6-((1H-indol-3-yl)methyl)-9-(tert-butoxycarbonyl)-14,17-bis(4-diazo-3-oxobutyl)-2-methyl-4,7,12,15-tetraoxo-2,5,8,13,16-pentaazaoctadecan-18-oate (P4)

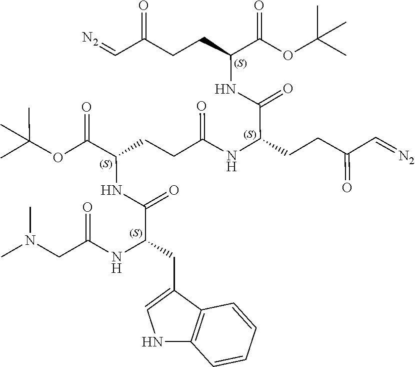

Compound 7 (270 mg, 0.359 mmol, 1 equiv) was dissolved in anhydrous DCM (5 mL), 2,5-dioxopyrrolidin-1-yl dimethylglycinate (144 mg, 0.718 mmol, 2.0 equiv) was added and the mixture was stirred at room temperature under nitrogen atmosphere for 1 h. DCM (300 mL) was added and the organic phase was washed with sat. NaHCO₃ (2×150 mL) and brine (150 mL), dried over anhydrous MgSO₄ and solvent was evaporated. Crude product was purified by LC on silica (DCM/MeOH, 15:1 to 10:1) and product P4 was isolated as a yellow solid (262 mg, 87%).

¹H NMR (CDCl₃): 1.44 (s, 9H), 1.49 (s, 9H), 1.81-2.13 (m, 5H), 2.17 (s, 6H), 2.20-2.32 (m, 3H), 2.32-2.62 (m, 4H), 2.89 (d, J=16.3 Hz, 1H), 3.01 (d, J=16.3 Hz, 1H), 3.23-3.41 (m, 2H), 4.36 (q, J=7.2 Hz, 1H), 4.42 (dt, J=8.0, 3.9 Hz, 1H), 4.49 (td, J=8.1, 4.5 Hz, 1H), 4.79 (q, J=6.8 Hz, 1H), 5.35 (bs, 1H), 5.37 (bs, 1H), 7.07 (d, J=7.7 Hz, 1H), 7.13 (ddd, J=8.1, 7.0, 1.1 Hz, 1H), 7.17-7.23 (m, 2H), 7.26 (bs, 1H), 7.30-7.34 (m, 1H), 7.36-7.40 (m, 1H), 7.68 (d, J=7.9 Hz, 1H), 7.74 (d, J=7.2 Hz, 1H), 8.67 (s, 1H). ESI-MS: 837.716 ([M+H]⁺)

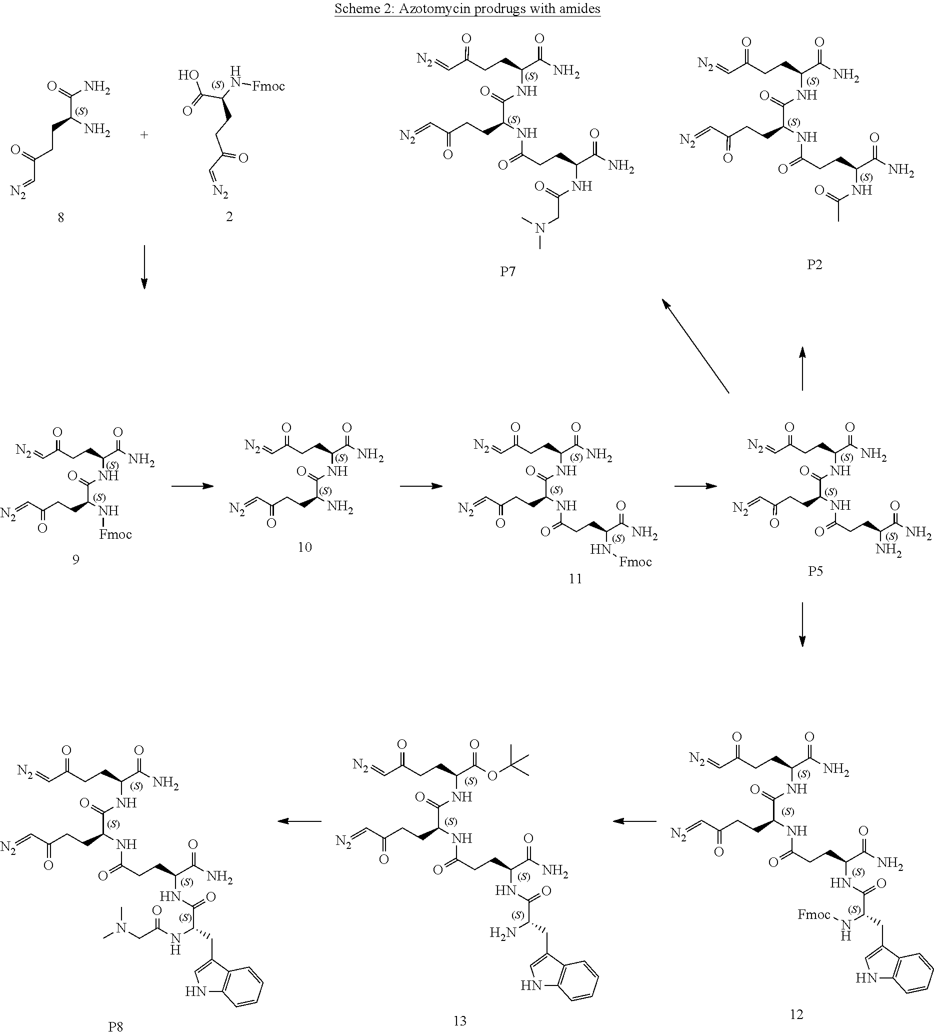

### (9H-Fluoren-9-yl)methyl ((S)-1-(((S)-1-amino-6-diazo-1,5-dioxohexan-2-yl)amino)-6-diazo-1,5-dioxohexan-2-yl)carbamate (9)

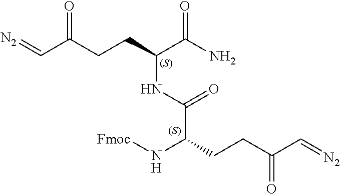

Fmoc-L-DON-OH (1.95 g, 4.96 mmol, 1.1 equiv) and HATU (1.97 g, 5.18 mmol, 1.15 equiv) were dissolved in anhydrous DMF (30 mL), the mixture was cooled to 0° C. and DIPEA (2.40 mL, 13.5 mmol, 3 equiv) was added. The mixture was stirred for 5 minutes and solution of H-L-DON-CONH₂ (746 mg, 4.38 mmol, 1 equiv) in anhydrous DMF (17 mL) was added. The resulting mixture was stirred for 30 minutes at 0° C. and 2 h at rt. DMF was evaporated and the crude product was purified by LC on silica (DCM/MeOH, 20:1 to 15:1) and product 9 was obtained as a yellow solid (1.46 g) in 61% yield.

¹H NMR (DMSO-d₆): 1.73-1.80 (m, 2H), 1.85-1.96 (m, 2H), 2.23-2.42 (m, 4H), 3.95-4.02 (m, 1H), 4.16-4.30 (m, 4H), 6.01 (bs, 2H), 7.05-7.08 (m, 1H), 7.30-7.43 (m, 5H), 7.54-7.58 (m, 1H), 7.71-7.74 (m, 2H), 7.86-7.95 (m, 3H). ESI-MS: 568.320 ([M+Na]⁺).

### (S)-2-Amino-N—((S)-1-amino-6-diazo-1,5-dioxohexan-2-yl)-6-diazo-5-oxohexanamide (10)

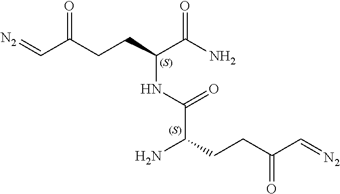

Compound 9 (1.46 g, 2.68 mmol, 1 equiv) was dissolved in the mixture of anhydrous solvents DCM/DMF (20+8 mL), diethylamine (2.80 mL, 27.0 mmol, 10 equiv) was added and the mixture was stirred at room temperature under nitrogen atmosphere for 1 h. Volatiles were removed under vacuo and the crude product 10 was immediately used to the following step without purification.

### (9H-Fluoren-9-yl)methyl ((S)-1-amino-5-(((S)-1-(((S)-1-amino-6-diazo-1,5-dioxohexan-2-yl)amino)-6-diazo-1,5-dioxohexan-2-yl)amino)-1,5-dioxopentan-2-yl)carbamate (11)

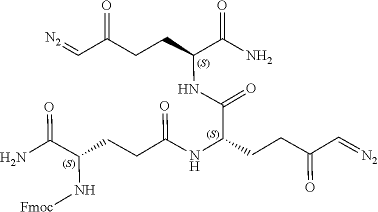

Fmoc-L-Glu(H)—CONH₂ (1.08 g, 2.94 mmol, 1.1 equiv) and HATU (1.17 g, 3.08 mmol, 1.15 equiv) were dissolved in anhydrous DMF (35 mL), the mixture was cooled to 0° C. and DIPEA (1.40 mL, 8.03 mmol, 3.0 equiv) was added. After 5 minutes solution of compound 10 (865 mg, 2.68 mmol, 1.0 equiv) in anhydrous DMF (15 mL) was added and the mixture was stirred for 30 minutes at 0° C. and overnight (16 h) at room temperature. DMF was evaporated and the crude product was purified by reverse phase HPLC (AcN/H₂O) and product 11 was obtained as a yellow solid (1.12 g, 62% yield over 2 steps).

¹H NMR (DMSO-d₆): 1.68-1.96 (m, 6H), 2.18-2.22 (m, 2H), 2.29-2.36 (m, 4H), 3.92 (d, J=6.6 Hz, 1H), 4.12-4.27 (m, 5H), 6.01 (bs, 2H), 7.04-7.07 (m, 2H), 7.26-7.52 (m, 7H), 7.73 (d, J=7.5 Hz, 2H), 7.88-7.95 (m, 3H), 8.03 (d, J=7.5 Hz, 1H). ESI-MS: 696.397 ([M+Na]⁺).

### (S)-4-Amino-N1-((S)-1-(((S)-1-amino-6-diazo-1,5-dioxohexan-2-yl)amino)-6-diazo-1,5-dioxohexan-2-yl)pentanediamide (P5)

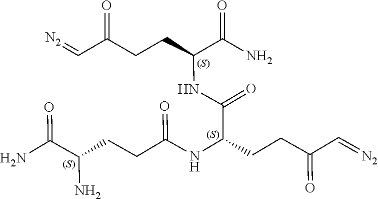

Compound 11 (1.12 mg, 1.66 mmol, 1 equiv) was dissolved in anhydrous DMF (25 mL), diethylamine (5 mL) was added and the mixture was stirred at room temperature under nitrogen atmosphere for 1 h. Volatiles were removed under vacuo and the residue was purified by LC on silica (DCM/MeOH, 2:1+1% Et₃N). Product P5 was isolated as a yellow solid (590 mg) in 79% yield.

¹H NMR (DMSO-d₆): 1.62 (dq, J=13.3, 7.8 Hz, 1H), 1.69-1.82 (m, 3H), 1.83-2.00 (m, 3H), 2.16-2.25 (m, 2H), 2.26-2.42 (m, 4H), 3.10 (dd, J=7.8, 5.3 Hz, 1H), 4.11-4.24 (m, 2H), 6.04 (2×CH, bs, 2H), 6.97 (bs, 1H), 7.06 (bs, 1H), 7.28 (bs, 1H), 7.32 (bs, 1H), 7.94 (d, J=8.1 Hz, 1H), 8.08 (d, J=7.4 Hz, 1H). ESI-MS: 452.302 ([M+H]⁺).

### (S)-4-Acetamido-N1-((S)-1-(((S)-1-amino-6-diazo-1,5-dioxohexan-2-yl)amino)-6-diazo-1,5-dioxohexan-2-yl)pentanediamide (P6)

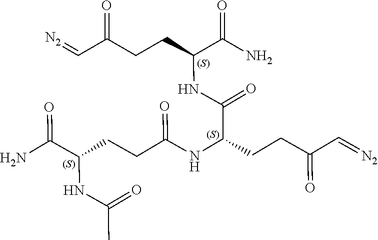

Compound P5 (175 g, 0.388 mmol, 1 equiv) was dissolved in anhydrous DCM (3 mL) and pyridine (94 μL, (S 1.16 mmol, 3.0 equiv) followed by acetanhydride (73 μL, 0.775 mmol, 2.0 equiv) were added. The reaction mixture was stirred for 1 h at room temperature under inert nitrogen atmosphere. Volatiles were removed under vacuo and the residue was purified by LC on silica (DCM/MeOH, 5:1+1% Et₃N). Product P6 was isolated as a yellow solid (114 mg) in 60% yield.

¹H NMR (DMSO-d₆): 1.72 (tq, J=13.5, 7.4 Hz, 3H), 1.85 (s, 3H), 1.86-1.98 (m, 3H), 2.16 (t, J=8.0 Hz, 2H), 2.24-2.40 (m, 4H), 4.07-4.22 (m, 3H), 6.03 (2×CH, bs, 2H), 7.01 (bs, 1H), 7.06 (bs, 1H), 7.29 (bs, 1H), 7.33 (bs, 1H), 7.88-7.96 (m, 2H), 8.04 (d, J=7.4 Hz, 1H). ESI-MS: 516.251 ([M+Na]⁺).

### (S)—N1-((S)-1-(((S)-1-Amino-6-diazo-1,5-dioxohexan-2-yl)amino)-6-diazo-1,5-dioxohexan-2-yl)-4-(2-(dimethylamino)acetamido)pentanediamide (P7)

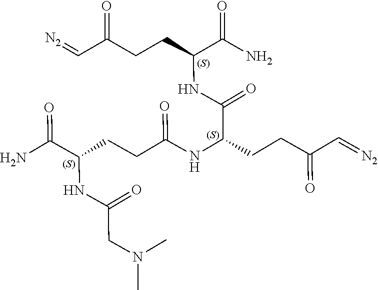

Compound P5 (140 mg, 0.310 mmol, 1 equiv) was dissolved in anhydrous DMF (5 mL) and 2,5-dioxopyrrolidin-1-yl dimethylglycinate (75 mg, 0.372 mmol, 1.2 equiv) was added and the mixture was stirred at room temperature under nitrogen atmosphere for 3 h. DMF was evaporated and crude product was purified by LC on silica (DCM/MeOH, 5:1+1% Et₃N) and product P7 was isolated as a yellow solid (140 mg, 84%).

¹H NMR (DMSO-d₆): 1.67-1.83 (m, 3H), 1.83-1.98 (m, 3H), 2.14 (t, J=7.9 Hz, 2H), 2.22 (s, 6H), 2.26-2.41 (m, 4H), 2.85-2.94 (m, 2H), 4.08-4.22 (m, 2H), 4.25 (td, J=8.0, 5.2 Hz, 1H), 6.02 (2×CH, bs, 2H), 7.05 (bs, 1H), 7.14 (bs, 1H), 7.28 (bs, 1H), 7.48 (bs, 1H), 7.70 (d, J=8.3 Hz, 1H), 7.91 (d, J=8.0 Hz, 1H), 8.06 (d, J=7.5 Hz, 1H). ESI-MS: 537.345 ([M+H]⁺).

### (9H-Fluoren-9-yl)methyl ((S)-1-(((S)-1-amino-5-(((S)-1-(((S)-1-amino-6-diazo-1,5-dioxohexan-2-yl)amino)-6-diazo-1,5-dioxohexan-2-yl)amino)-1,5-dioxopentan-2-yl)amino)-3-(1H-indol-3-yl)-1-oxopropan-2-yl)carbamate (12)

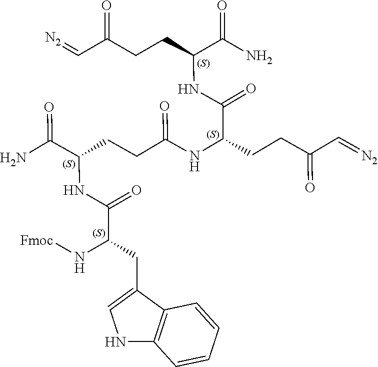

Fmoc Fmoc-L-Trp-OH (208 mg, 0.488 mmol, 1.1 equiv) and HATU (194 mg, 0.510 mmol, 1.15 equiv) were dissolved in anhydrous DMF (6 mL), the mixture was cooled to 0° C. and DIPEA (232 μL, 1.33 mmol, 3.0 equiv) was added. After 5 minutes solution of compound P5 (200 mg, 0.444 mmol, 1.0 equiv) in anhydrous DMF (4 mL) was added and the mixture was stirred for 30 minutes at 0° C. and for 1.5 h at room temperature. DMF was evaporated and the residue was purified by reverse phase HPLC (AcN/H₂O) and product 12 was obtained as a yellow solid (356 mg, 94% yield).

¹H NMR (DMSO-d₆): 1.66-2.03 (m, 4H), 2.20 (t, J=8.0 Hz, 2H), 2.26-2.37 (m, 4H), 2.41-2.46 (m, 2H), 2.94-3.21 (m, 2H), 4.11-4.25 (m, 6H), 4.27-4.36 (m, 1H), 6.00 (bs, 2H), 6.97 (t, J=7.4 Hz, 1H), 7.01-7.11 (m, 3H), 7.15-7.20 (m, 1H), 7.21-7.35 (m, 4H), 7.39 (q, J=7.0 Hz, 2H), 7.52 (d, J=8.2 Hz, 1H), 7.58-7.71 (m, 3H), 7.86 (d, J=7.6 Hz, 2H), 7.92-8.02 (m, 4H), 8.07 (d, J=8.0 Hz, 1H). ESI-MS: 882.621 ([M+Na]⁺).

### (S)-4-((S)-2-Amino-3-(1H-indol-3-yl)propanamido)-N1-((S)-1-(((S)-1-amino-6-diazo-1,5-dioxohexan-2-yl)amino)-6-diazo-1,5-dioxohexan-2-yl)pentanediamide (13)

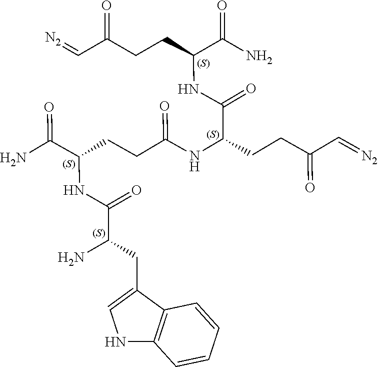

Compound 12 (356 mg, 0.414 mmol, 1 equiv) was dissolved in anhydrous DMF (8 mL), diethylamine (856 μL, 8.28 mmol, 20 equiv) was added and the mixture was stirred at room temperature under nitrogen atmosphere for 1 h. Volatiles were removed under vacuo and the residue was purified by LC on silica (DCM/MeOH, 2:1+1% Et₃N). Product 13 was isolated as a yellow solid (222 mg) in 84% yield.

¹H NMR (DMSO-d₆): 1.64-1.83 (m, 3H), 1.87-2.01 (m, 3H), 2.13-2.28 (m, 4H), 2.28-2.43 (m, 4H), 2.71 (d, J=15.4 Hz, 1H), 2.86 (d, J=15.4 Hz, 1H), 4.07-4.30 (m, 2H), 4.50-4.65 (m, 2H), 6.00 (bs, 1H), 6.05 (bs, 1H), 6.98 (t, J=7.3 Hz, 1H), 7.00-7.24 (m, 3H), 7.22-7.30 (m, 3H), 7.58 (d, J=7.6 Hz, 1H), 7.70 (d, J=7.2 Hz, 1H), 7.92 (d, J=7.6 Hz, 1H), 8.04 (d, J=7.1 Hz, 1H), 8.19 (d, J=7.6 Hz, 1H), 10.42 (bs, 1H). ESI-MS: 638.462 ([M+H]⁺).

### (S)—N1-((S)-1-(((S)-1-Amino-6-diazo-1,5-dioxohexan-2-yl)amino)-6-diazo-1,5-dioxohexan-2-yl)-4-((S)-2-(2-(dimethylamino)acetamido)-3-(1H-indol-3-yl)propanamido)pentanediamide (P8)

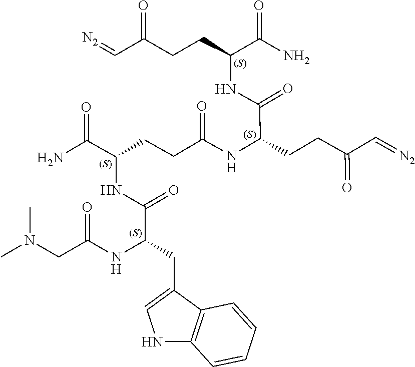

Compound 13 (222 mg, 0.348 mmol, 1 equiv) was dissolved in anhydrous DMF (2.5 mL), 2,5-dioxopyrrolidin-1-yl dimethylglycinate (140 mg, 0.696 mmol, 2.0 equiv) was added and the mixture was stirred at room temperature under nitrogen atmosphere for 1 h. DMF was evaporated and the crude product was purified by reverse phase HPLC (ACN/H₂O) and product P8 was isolated as a yellow solid (133 mg, 53%).

¹H NMR (DMSO-d₆): 1.66-1.84 (m, 3H), 1.84-1.99 (m, 3H), 2.01 (s, 6H), 2.13-2.25 (m, 2H), 2.26-2.41 (m, 4H), 2.70 (d, J=15.5 Hz, 1H), 2.83 (d, J=15.5 Hz, 1H), 2.98-3.09 (m, 1H), 3.14-3.24 (m, 1H), 4.05-4.28 (m, 3H), 4.55-4.69 (m, 1H), 6.03 (2×CH, bs, 2H), 6.96 (t, J=7.2 Hz, 1H), 7.01-7.19 (m, 4H), 7.24-7.36 (m, 3H), 7.57 (d, J=7.6 Hz, 1H), 7.69 (d, J=7.2 Hz, 1H), 7.94 (d, J=7.7 Hz, 1H), 8.01 (d, J=7.0 Hz, 1H), 8.17 (d, J=7.7 Hz, 1H), 10.80 (bs, 1H). ESI-MS: 723.491 ([M+H]⁺).

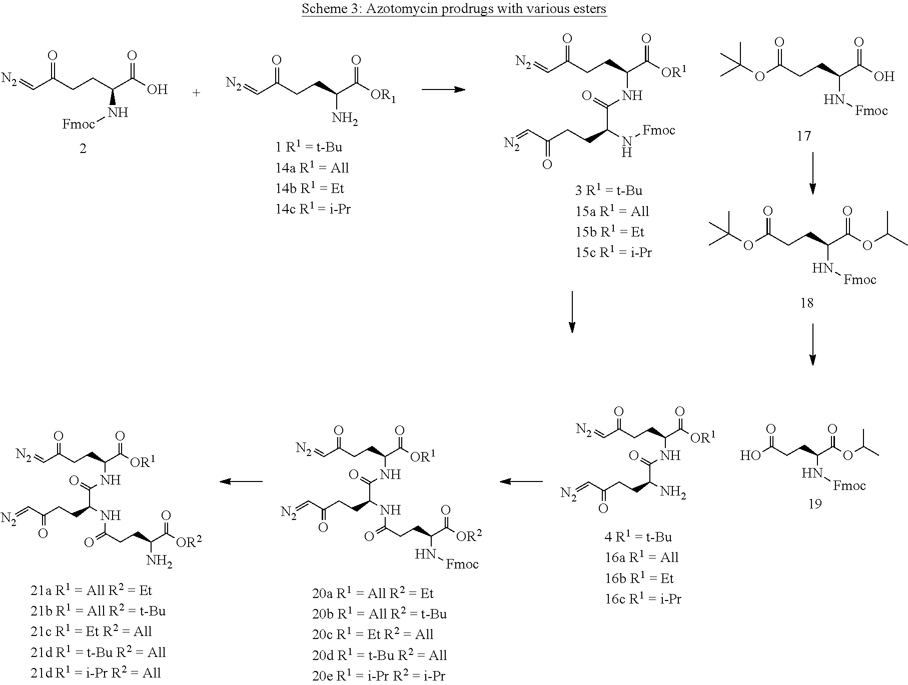

### 1.2.1 General Procedure for Compounds 15a-c

Fmoc-L-DON-OH (1.50 g, 3.81 mmol, 1.1 equiv) and HATU (1.52 g, 3.98 mmol, 1.15 equiv) were suspended in anhydrous DCM (40 mL). The mixture was cooled down to 0° C. and DIPEA (1.81 mL, 10.4 mmol, 3 equiv) was added. The mixture was stirred for 5 minutes and the solution of H-L-DON-OR¹ (3.47 mmol, 1 equiv) in anhydrous DCM (10 mL) was added dropwise. The resulting mixture was stirred for 30 minutes at 0° C. and 16 h at room temperature. Volatiles were evaporated, the residue was dissolved in EtOAc (200 mL) and washed with sat. NaHCO₃ (150 mL), H₂O (80 mL) and brine (80 mL). The organic layer was dried over anhydrous MgSO₄ and DCM was evaporated. The crude product was purified by LC on silica (DCM/EtOAc).

Allyl (S)-2-((S)-2-((((9H-fluoren-9-yl)methoxy)carbonyl)amino)-6-diazo-5-oxohexanamido)-6-diazo-5-oxohexanoate (15a)

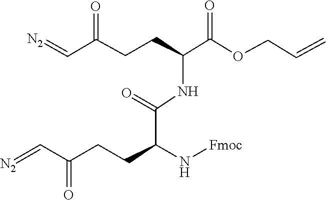

H-L-DON-OA11 (14a) (732 mg), purified by LC on silica (DCM/EtOAc, 3:5) to afford the product 15a (1.55 g, 76%) as a yellow solid.

¹H NMR (DMSO-d₆): 1.71-1.93 (m, 3H), 1.96-2.08 (m, 2H), 2.26-2.46 (m, 4H), 3.97-4.08 (m, 1H), 4.17-4.33 (m, 4H), 4.53-4.62 (m, 2H), 5.18-5.23 (m, 1H), 5.26-5.34 (m, 1H), 5.87-5.97 (m, 1H), 5.98-6.09 (m, 1H), 7.29-7.44 (m, 4H), 7.53-7.60 (m, 1H), 7.70-7.78 (m, 2H), 7.90 (d, J=7.9 Hz, 2H), 8.35-8.45 (m, 1H). ESI-MS: 609.3 ([M+Na]⁺).

### Ethyl (S)-2-((S)-2-((((9H-fluoren-9-yl)methoxy)carbonyl)amino)-6-diazo-5-oxohexanamido)-6-diazo-5-oxohexanoate (15b) (124-189)

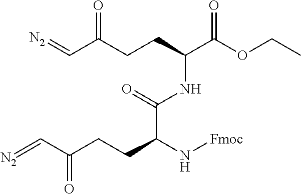

H-L-DON-Oet (14b) (690 mg), purified by LC on silica (DCM/EtOAc, 1:2) to afford the product 15b (677 mg, 34%) as a yellow oil. ESI-MS: 597.3 ([M+Na]⁺).

### Isopropyl (S)-2-((S)-2-((((9H-fluoren-9-yl)methoxy)carbonyl)amino)-6-diazo-5-oxohexanamido)-6-diazo-5-oxohexanoate (15c)

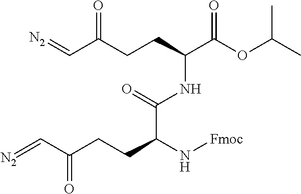

H-L-DON-OiPr (14c) (739 mg), purified by LC on silica (DCM/EtOAc, 1:1) to afford the product 15c (1.22 g, 60%) as a yellow solid.

¹H NMR (CDCl₃): 1.20-1.26 (m, 6H), 1.90-2.00 (m, 2H), 2.08-2.23 (m, 2H), 2.30-2.41 (m, 2H), 2.42-2.52 (m, 2H), 4.20 (t, J=7.0 Hz, 2H), 4.31-4.40 (m, 2H), 4.44 (dd, J=8.9, 4.6 Hz, 1H), 4.95-5.05 (m, 1H), 5.26-5.31 (m, 1H), 5.32-5.41 (m, 1H), 6.01-6.12 (m, 1H), 7.34 (dt, J=42.4, 7.5 Hz, 4H), 7.55-7.62 (m, 3H), 7.74 (d, J=7.5 Hz, 2H). ESI-MS: 611.3 ([M+Na]).

General procedure for compounds 16a-c: Compound 15c (1.70 mmol, 1 equiv) was dissolved in anhydrous DCM (7 mL) and diethylamine (17.0 mmol, 10 equiv) was added. The mixture was stirred for 2 h at rt. Volatiles were evaporated in vacuo and the crude product was used to the further reaction without any purification because of significant instability of the product.

### Allyl (S)-2-((S)-2-amino-6-diazo-5-oxohexanamido)-6-diazo-5-oxohexanoate (16a) ESI-MS: 387.2 ([M+Na]⁺)

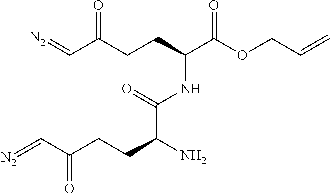

### Ethyl (S)-2-((S)-2-amino-6-diazo-5-oxohexanamido)-6-diazo-5-oxohexanoate (16b) ESI-MS: 353.2 ([M+H]⁺)

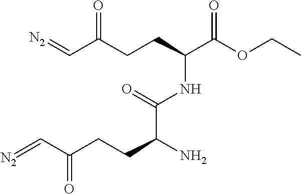

### Isopropyl (S)-2-((S)-2-amino-6-diazo-5-oxohexanamido)-6-diazo-5-oxohexanoate (16c) ESI-MS: 367.2 ([M+H]⁺)

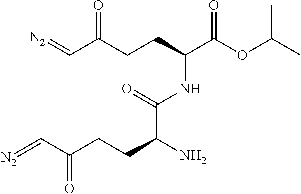

### 5-(tert-Butyl) 1-isopropyl (((9H-fluoren-9-yl)methoxy)carbonyl)-L-glutamate (18)

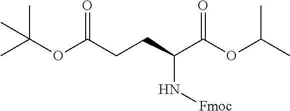

Fmoc-L-Glu(t-Bu)-OH (17) (5.0 g, 11.8 mmol, 1 equiv) was dissolved in anhydrous DCM (50 mL) and i-PrOH (4.50 mL, 58.7 mmol, 5 equiv) was added. CDI (3.81 g, 23.5 mmol, 2 equiv) was added in one portion and the resulting mixture was stirred for 24 h at rt. Volatiles were evaporated in vacuo, the crude mixture was diluted with DCM (100 mL) and washed with 10% KHSO₄ (100 mL) and brine (50 mL). The organic layer was dried over MgSO₄, concentrated in vacuo and purified by liquid chromatography on silica (hexane/EtOAc, grad. 7:1 to 4:1) to afford the product 18 (5.16 g, 94%) as a colorless oil.

¹H NMR (CDCl₃): 1.22-1.29 (m, 6H), 1.45 (s, 9H), 1.90-1.99 (m, 1H), 2.11-2.19 (m, 1H), 2.29-2.39 (m, 2H), 4.12 (q, J=7.1 Hz, 1H), 4.23 (t, J=7.1 Hz, 1H), 4.32-4.45 (m, 2H), 5.07 (hept, J=6.1 Hz, 1H), 5.45 (d, J=8.1 Hz, 1H), 7.36 (dt, J=42.1, 7.4 Hz, 4H), 7.54-7.66 (m, 2H), 7.77 (d, J=7.6 Hz, 2H). ESI-MS: 468.2 ([M+H]⁺).

### (S)-4-((((9H-Fluoren-9-yl)methoxy)carbonyl)amino)-5-isopropoxy-5-oxopentanoic acid (19)

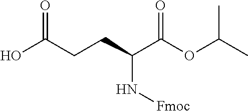

Compound 18 (5.16 g, 11.04 mmol, 1 equiv) was dissolved in anhydrous DCM (25 mL), cooled to 0° C. and TFA (25 mL) was added dropwise. The reaction mixture was stirred for 2 h at rt and then concentrated in vacuo. The crude mixture was triturated with hexane, filtrated and dried in vacuo. The product 19 (4.40 g, 97%) was afforded as a colorless solid.

¹H NMR (CDCl₃): 1.19-1.39 (m, 6H), 1.82-2.60 (m, 4H), 4.00-4.57 (m, 4H), 5.00-5.19 (m, 1H), 5.51-5.62 (m, 1H), 7.18-7.48 (m, 4H), 7.55-7.62 (m, 2H), 7.72-7.89 (m, 2H), 9.50 (s, 1H). ESI-MS: 412.2 ([M+H]⁺).

General procedure for compounds 20a-e: Fmoc-Glu(OH)—OR² (1.1 equiv) and HATU (1.2 equiv) were suspended in anhydrous DCM and the mixture was cooled to 0° C. DIPEA (3 equiv) was added and the reaction mixture was stirred for 10 min at the same temperature. The solution of compound 16a-c (1 equiv) in anhydrous DCM was added dropwise. The resulting mixture was stirred for 30 min at 0° C. and for 16 h at rt, and then concentrated in vacuo. The work up (if any) and purification depended on each individual reaction.

### Allyl (5S,10S,13S)-10,13-bis(4-diazo-3-oxobutyl)-5-(ethoxycarbonyl)-1-(9H-fluoren-9-yl)-3,8,11-trioxo-2-oxa-4,9,12-triazatetradecan-14-oate (20a)

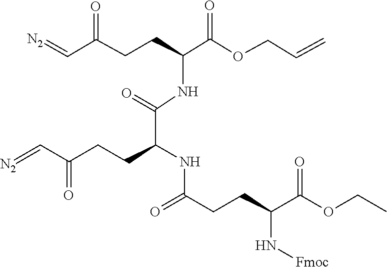

Fmoc-L-Glu(OH)-OEt (745 mg, 1.87 mmol) and HATU (778 mg, 2.05 mmol) in DCM (15 mL), DIPEA (891 μL, 5.11 mmol), amine 16a (621 mg, 1.71 mmol) in DCM (7 mL); purified by chromatography on silica (DCM/MeOH, 30:1) to afford product 20a (1.02 g, 80%) as a yellow solid. ESI-MS: 766.3 ([M+Na]⁺).

### Allyl (5S,10S,13S)-5-(tert-butoxycarbonyl)-10,13-bis(4-diazo-3-oxobutyl)-1-(9H-fluoren-9-yl)-3,8,11-trioxo-2-oxa-4,9,12-triazatetradecan-14-oate (20b)

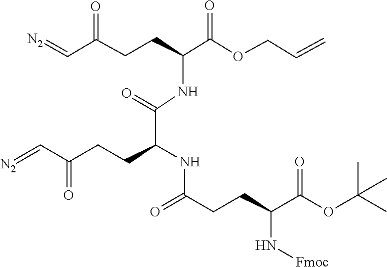

Fmoc-L-Glu(OH)-OtBu (374 mg, 0.879 mmol) and HATU (349 mg, 0.918 mmol) in DCM (8 mL), DIPEA (417 μL, 2.40 mmol), amine 16a (291 mg, 0.800 mmol) in DCM (6 mL); purified by chromatography on silica (DCM/MeOH, 30:1) to afford product 20b (425 mg, 69%) as a yellow oil.

¹H NMR (DMSO-d₆) 1.39 (s, 9H), 1.67-2.06 (m, 6H), 2.17-2.25 (m, 2H), 2.27-2.46 (m, 4H), 3.85-3.95 (m, 1H), 4.18-4.35 (m, 6H), 4.56 (dt, J=5.5, 1.4 Hz, 2H), 5.20 (dq, J=10.5, 1.4 Hz, 1H), 5.30 (dq, J=17.2, 1.6 Hz, 111), 5.95-6.10 (m, 2H), 7.30-7.45 (m, 4H), 7.71 (t, J=8.6 Hz, 3H), 7.90 (d, J=7.5 Hz, 2H), 8.03 (d, J=7.9 Hz, 1H), 8.40 (d, J=7.4 Hz, 1H). ESI-MS: 794.3 ([M+Na]⁺).

### Ethyl (5S,10S,13S)-5-((allyloxy)carbonyl)-10,13-bis(4-diazo-3-oxobutyl)-1-(9H-fluoren-9-yl)-3,8,11-trioxo-2-oxa-4,9,12-triazatetradecan-14-oate (20c)

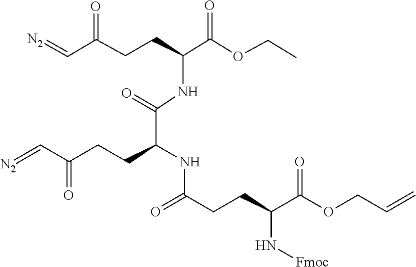

Fmoc-L-Glu(OH)-OA11 (689 mg, 1.68 mmol) and HATU (698 mg, 1.84 mmol) in DCM (13 mL), DIPEA (799 μL, 4.59 mmol), amine 16b (539 mg, 1.53 mmol) in DCM (6 mL); purified by chromatography on silica (DCM/MeOH, 30:1) to afford product 20c (1.06 g, 93%) as a yellow oil.

¹H NMR (CDCl₃): 1.26 (t, J=7.1 Hz, 3H), 1.92-2.05 (m, 4H), 2.08-2.31 (m, 3H), 2.32-2.45 (m, 3H), 2.45-2.62 (m, 4H), 4.17 (q, J=7.1 Hz, 2H), 4.18-4.24 (m, 1H), 4.33-4.44 (m, 3H), 4.48 (td, J=8.4, 4.9 Hz, 1H), 4.65 (d, J=5.6 Hz, 1H), 5.26 (d, J=10.4 Hz, 1H), 5.31-5.42 (m, 2H), 5.81 (d, J=7.9 Hz, 1H), 5.85-6.05 (m, 3H), 6.82 (d, J=6.3 Hz, 1H), 7.30-7.42 (m, 4H), 7.58-7.64 (m, 2H), 7.75-7.78 (m, 2H). ESI-MS: 766.4 ([M+Na]⁺).

### tert-Butyl (5S,10S,13S)-5-((allyloxy)carbonyl)-10,13-bis(4-diazo-3-oxobutyl)-1-(9H-fluoren-9-yl)-3,8,11-trioxo-2-oxa-4,9,12-triazatetradecan-14-oate (20d)

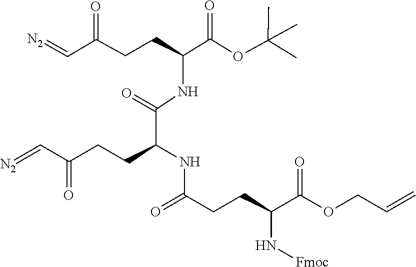

Fmoc-L-Glu(OH)-OA11 (485 mg, 1.18 mmol) and HATU (491 mg, 1.29 mmol) in DCM (10 mL), DIPEA (563 μL, 3.23 mmol), amine 4 (410 mg, 1.08 mmol) in DCM (10 mL); purified by chromatography on silica (DCM/MeOH, 30:1) to afford product 20d (640 mg, 59%) as a yellow oil.

¹H NMR (DMSO-d₆) 1.38 (s, 9H), 1.66-1.83 (m, 3H), 1.85-2.08 (m, 3H), 2.20-2.44 (m, 7H), 4.01-4.13 (m, 2H), 4.20-4.36 (m, 4H), 4.55-4.62 (m, 2H), 5.15-5.22 (m, 1H), 5.30 (dq, J=17.3, 1.6 Hz, 1H), 5.99 (bs, 1H), 6.06 (bs, 1H), 7.38 (dt, J=42.4, 7.4 Hz, 4H), 7.71 (d, J=7.4 Hz, 2H), 7.85 (d, J=8.0 Hz, 1H), 7.90 (d, J=7.5 Hz, 2H), 8.02 (d, J=7.9 Hz, 1H), 8.25 (d, J=7.2 Hz, 1H). ESI-MS: 744.3 ([M-N2+H]⁺).

### Isopropyl (5S,10S,13S)-10,13-bis(4-diazo-3-oxobutyl)-1-(9H-fluoren-9-yl)-5-(isopropoxycarbonyl)-3,8,11-trioxo-2-oxa-4,9,12-triazatetradecan-14-oate (20e)

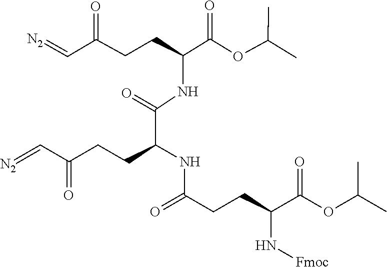

Fmoc-L-Glu(OH)-OiPr (19) (1.46 g, 3.55 mmol) and HATU (1.47 g, 3.87 mmol) in DCM (30 mL), DIPEA (1.69 mL, 9.68 mmol), amine 16c (1.18 g, 3.23 mmol) in DCM (15 mL); diluted with EtOAc (250 mL) and washed with sat. NaHCO₃ (130 mL), 50% solution of NaCl in water (120 mL) and with brine (90 mL). The organic layer was dried over MgSO₄, concentrated in vacuo and purified by chromatography on silica (DCM/MeOH, 30:1) to afford product 20e (370 mg, 14%) as a yellow oil.

¹H NMR (CDCl₃): 1.22-1.30 (m, 12H), 1.90-2.05 (m, 3H), 2.07-2.28 (m, 3H), 2.31 (t, J=7.1 Hz, 2H), 2.34-2.47 (m, 2H), 2.47-2.68 (m, 2H), 4.23 (t, J=7.1 Hz, 1H), 4.28-4.35 (m, 1H), 4.35-4.43 (m, 3H), 4.46 (td, J=8.1, 4.7 Hz, 1H), 4.98-5.12 (m, 2H), 5.29 (bs, 1H), 5.34 (bs, 1H), 5.70 (d, J=7.8 Hz, 1H), 6.77 (d, J=6.4 Hz, 1H), 7.29-7.44 (m, 5H), 7.57-7.64 (m, 2H), 7.77 (d, J=7.5 Hz, 2H). ESI-MS: 782.3 ([M+Na]⁺).

General procedure for compounds 21a-d and P9: Compound 20a-e (1 equiv) was dissolved in anhydrous DCM and diethylamine (10 equiv) was added. The reaction mixture was stirred for 4 h at rt and then concentrated in vacuo. The crude mixture was purified by chromatography on silica (DCM/MeOH, 10:1).

### Allyl (S)-2-((S)-2-((S)-4-amino-5-ethoxy-5-oxopentanamido)-6-diazo-5-oxohexanamido)-6-diazo-5-oxohexanoate (21a)

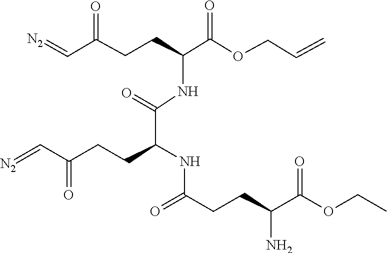

Compound 20a (1.0 g, 1.34 mmol) in DCM (7 mL), Et₂NH (1.4 mL, 13.4 mmol), product 21a (500 mg, 71%) as a yellow amorphous compound.

¹H NMR (CDCl₃): 1.28 (t, J=7.1 Hz, 3H), 1.90-2.03 (m, 3H), 2.12-2.30 (m, 4H), 2.37-2.52 (m, 6H), 3.07 (q, J=7.2 Hz, 2H), 4.17-4.26 (m, 2H), 4.32 (t, J=6.7 Hz, 1H), 4.44-4.49 (m, 1H), 4.54-4.65 (m, 2H), 5.18-5.27 (m, 1H), 5.26-5.35 (m, 1H), 5.44-5.50 (m, 2H), 5.82-5.93 (m, 1H), 7.40 (d, J=6.7 Hz, 1H), 7.68 (d, J=7.8 Hz, 1H). ESI-MS: 522.3 ([M+H]⁺).

### Allyl (S)-2-((S)-2-((S)-4-amino-5-(tert-butoxy)-5-oxopentanamido)-6-diazo-5-oxohexanamido)-6-diazo-5-oxohexanoate (21b)

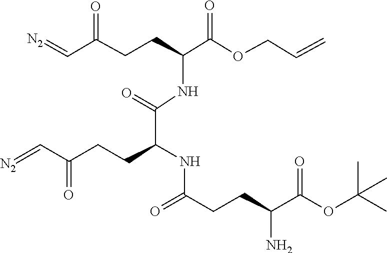

Compound 20b (423 mg, 0.548 mmol) in DCM (3 mL), Et₂NH (567 μL, 5.98 mmol), product 21b (180 mg, 60%) as a yellow amorphous compound.

¹H NMR (CDCl₃): 1.49 (s, 9H), 2.00-2.15 (m, 4H), 2.16-2.28 (m, 3H), 2.39-2.64 (m, 6H), 3.68-3.79 (m, 2H), 4.32-4.41 (m, 1H), 4.48-4.55 (m, 1H), 4.56-4.68 (m, 2H), 5.23-5.38 (m, 2H), 5.41-5.50 (m, 2H), 5.80-5.91 (m, 1H), 7.31-7.37 (m, 1H), 7.52 (d, J=8.8 Hz, 1H). ESI-MS: 550.3 ([M+H]⁺).

### Ethyl (S)-2-((S)-2-((S)-5-(allyloxy)-4-amino-5-oxopentanamido)-6-diazo-5-oxohexanamido)-6-diazo-5-oxohexanoate (21c)

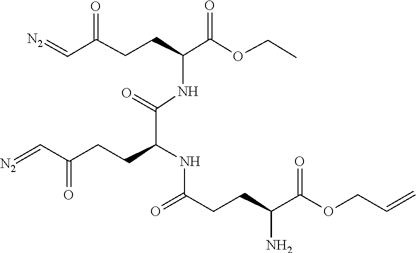

Compound 20c (1.09 g, 1.46 mmol) in DCM (8 mL), Et₂NH (1.52 mL, 14.6 mmol), product 21c (523 mg, 68%) as a yellow amorphous compound.

¹H NMR (CDCl₃): 1.26 (t, J=7.1 Hz, 3H), 1.97-2.11 (m, 4H), 2.13-2.32 (m, 3H), 2.32-2.39 (m, 1H), 2.40-2.47 (m, 2H), 2.50-2.63 (m, 3H), 2.70-2.79 (m, 1H), 4.12-4.20 (m, 2H), 4.22 (dd, J=8.2, 3.1 Hz, 1H), 4.34-4.41 (m, 1H), 4.46 (td, J=8.4, 4.5 Hz, 1H), 4.70-4.76 (m, 2H), 5.28-5.40 (m, 2H), 5.43-5.51 (m, 1H), 5.86-5.97 (m, 2H), 7.42-7.49 (m, 2H). ESI-MS: 522.3 ([M+H]⁺).

### tert-Butyl (S)-2-((S)-2-((S)-5-(allyloxy)-4-amino-5-oxopentanamido)-6-diazo-5-oxohexanamido)-6-diazo-5-oxohexanoate (21d)

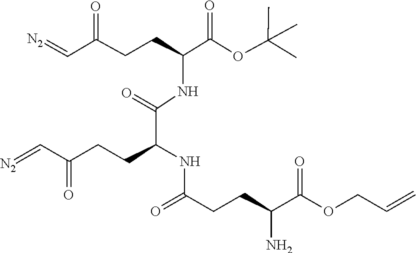

Compound 20d (634 mg, 0.821 mmol) in DCM (4 mL), Et₂NH (850 μL, 8.21 mmol), product 21d (360 mg, 80%) as a yellow amorphous compound.

¹H NMR (CDCl₃): 1.39 (s, 9H), 1.81-1.98 (m, 3H), 2.00-2.20 (m, 3H), 2.27-2.47 (m, 6H), 3.23-3.30 (m, 2H), 3.55-3.69 (m, 1H), 4.21-4.32 (m, 2H), 4.60 (d, J=5.9 Hz, 2H), 5.18-5.33 (m, 2H), 5.42-5.48 (m, 2H), 5.82-5.91 (m, 1H), 7.29-7.33 (m, 1H), 7.45-7.52 (m, 1H). ESI-MS: 550.3 ([M+H]⁺).

### Isopropyl (S)-2-((S)-2-((S)-4-amino-5-isopropoxy-5-oxopentanamido)-6-diazo-5-oxohexanamido)-6-diazo-5-oxohexanoate (P9)

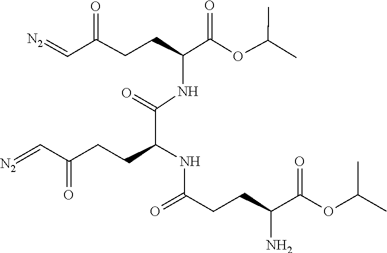

Compound 20e (365 mg, 0.480 mmol) in DCM (10 mL), NH Et₂NH (497 μL, 4.80 mmol), product P9 (220 mg, 85%) as a yellow amorphous compound.

¹H NMR (CDCl₃): 1.21-1.26 (m, 12H), 1.62-1.70 (m, 2H, 1.81-1.90 (m, 1H), 1.95-2.27 (m, 5H), 2.30-2.45 (m, 4H), 2.47-2.55 (m, 1H), 2.55-2.66 (m, 1H), 3.42 (dd, J=8.8, 4.9 Hz, 1H), 4.34-4.57 (m, 2H), 5.03 (hept, J=6.6 Hz, 2H), 5.26-5.42 (m, 2H), 6.99 (d, J=6.8 Hz, 1H), 7.51 (d, J=7.8 Hz, 1H). ESI-MS: 538.3 ([M+H]⁺).

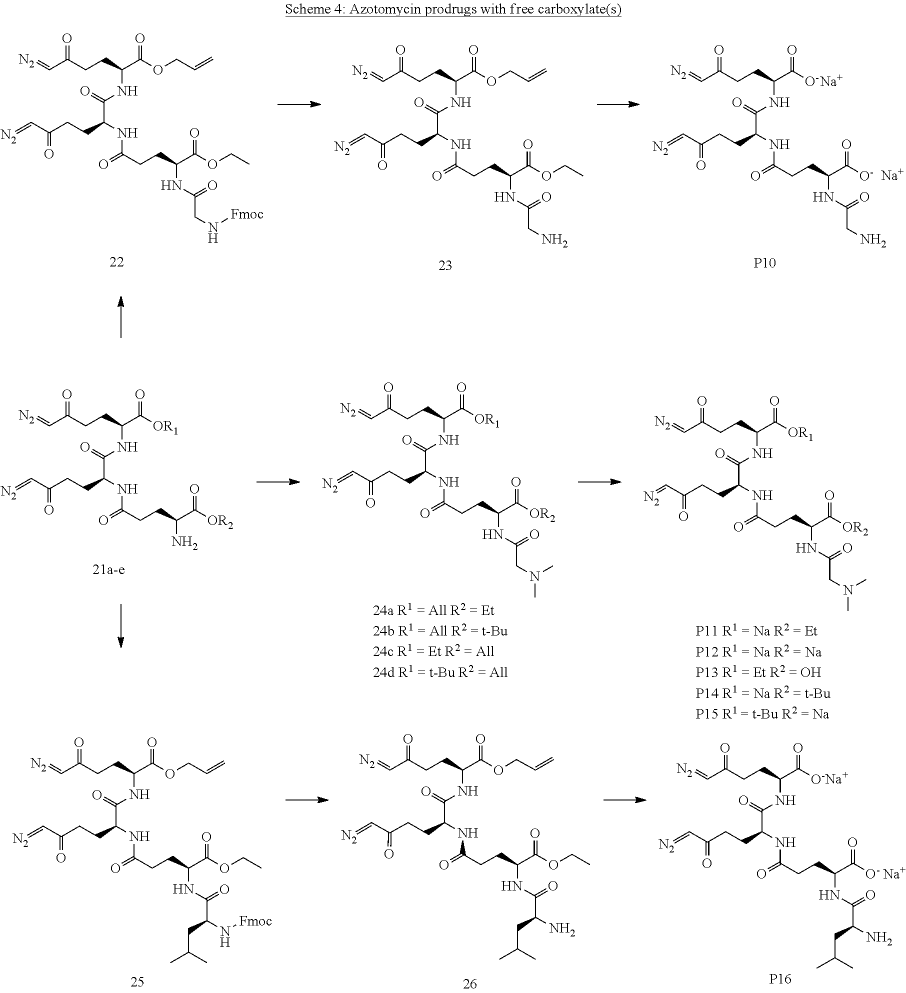

### Allyl (8S,13S,16S)-13,16-bis(4-diazo-3-oxobutyl)-8-(ethoxycarbonyl)-1-(9H-fluoren-9-yl)-3,6,11,14-tetraoxo-2-oxa-4,7,12,15-tetraazaheptadecan-17-oate (22)

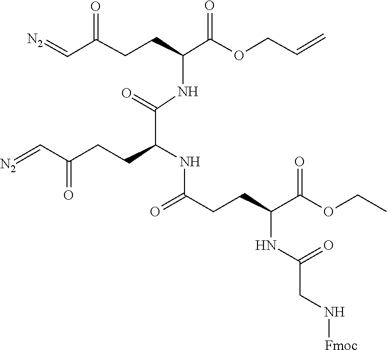

Fmoc-Gly-OH (94 mg, 0.316 mmol, 1.1 equiv) and HATU (126 mg, 0.330 mmol, 1.15 equiv) were suspended in anhydrous DCM (8 mL) and cooled down to 0° C. DIPEA (150 μL, 0.863 mmol, 3 equiv) was added and the mixture was stirred for 5 min at the same temperature. The solution of compound 21a (150 mg, 0.288 mmol, 1 equiv) in anhydrous DCM (6 mL) was added dropwise and the solution was stirred for 30 min at 0° C. and for 4 h at rt. The reaction mixture was concentrated in vacuo and purified by chromatography on silica (DCM/MeOH, 20:1) to afford the product 22 (159 mg, 69%) as a yellow solid. ESI-MS: 823.4 ([M+Na]⁺).

### Allyl (S)-2-((S)-2-((S)-4-(2-aminoacetamido)-5-ethoxy-5-oxopentanamido)-6-diazo-5-oxohexanamido)-6-diazo-5-oxohexanoate (23)

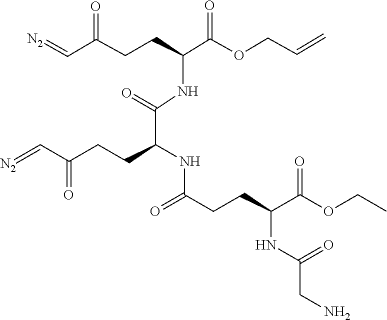

Compound 22 (157 mg, 0.196 mmol, 1 equiv) was dissolved in anhydrous DCM (2 mL) and diethylamine (193 μL, 10 equiv) was added. The reaction mixture was stirred at rt for 3 h and then concentrated in vacuo. The residue was purified by chromatography on silica (DCM/MeOH, grad. 5:1 to 1:1) to afford product 23 (54 mg, 48%).

¹H NMR (CDCl₃): 1.28 (t, J=7.1 Hz, 3H), 1.92-2.08 (m, 4H), 2.10-2.19 (m, 1H), 2.19-2.30 (m, 2H), 2.33 (t, J=7.2 Hz, 2H), 2.38-2.61 (m, 5H), 3.45-3.52 (m, 2H), 4.20 (q, J=7.1 Hz, 2H), 4.44 (q, J=6.9 Hz, 1H), 4.51-4.60 (m, 2H), 4.62 (dq, J=4.2, 1.4 Hz, 2H), 5.23-5.28 (m, 1H), 5.33 (dq, J=17.2, 1.4 Hz, 1H), 5.39-5.44 (m, 2H), 5.84-5.97 (m, 1H), 7.23 (d, J=6.0 Hz, 1H), 7.55 (d, J=7.5 Hz, 1H), 7.97 (d, J=8.1 Hz, 1H). ESI-MS: 579.3 ([M+H]⁺).

### Sodium (S)-2-((S)-2-((S)-4-(2-aminoacetamido)-4-carboxylatobutanamido)-6-diazo-5-oxohexanamido)-6-diazo-5-oxohexanoate (P10)

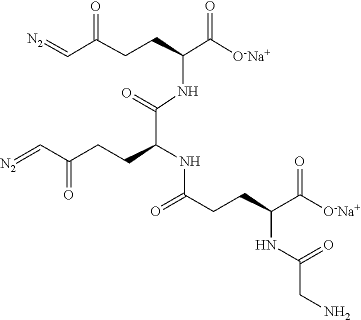

Compound 23 (23 mg, 0.040 mmol, 1 equiv) was dissolved in the mixture of THE (250 μL) and MeOH (150 μL) and 1M NaOH (240 μL, 0.239 mmol, 6 equiv) was added. The reaction mixture was stirred for 45 min at rt and then concentrated in vacuo. The residue was purified by preparative HPLC (grad. 0-20% MeCN/H₂O in 50 min) to afford product P10 (6 mg, 30%) as a light orange solid.

¹H NMR (D₂O): 1.85-1.98 (m, 3H), 2.08-2.19 (m, 3H), 2.31-2.42 (m, 4H), 2.47 (t, J=7.4 Hz, 2H), 3.78-3.83 (m, 2H), 4.13 (dd, J=8.6, 4.8 Hz, 1H), 4.17 (dd, J=9.0, 4.7 Hz, 1H), 4.28 (dd, J=8.5, 5.9 Hz, 1H), 5.83 (s, 1H), 5.88 (s, 1H). ESI-MS: 509.2 ([M−H]⁺).

### 1.22. General Procedure for Compounds 24a-d

Compound 21a-d (1 equiv) was dissolved in anhydrous DCM and 2,5-dioxopyrrolidin-1-yl dimethylglycinate (1.1 equiv) was added. The mixture was stirred at rt for 1.5 h and then diluted with DCM (70 mL) and washed with sat. NaHCO₃ (35 mL) and brine (35 mL). The organic layer was dried over MgSO₄ and concentrated in vacuo. The residue was purified by chromatography on silica (DCM/MeOH, 10:1).

### Allyl (6S,11S,14S)-11,14-bis(4-diazo-3-oxobutyl)-6-(ethoxycarbonyl)-2-methyl-4,9,12-trioxo-2,5,10,13-tetraazapentadecan-15-oate (24a)

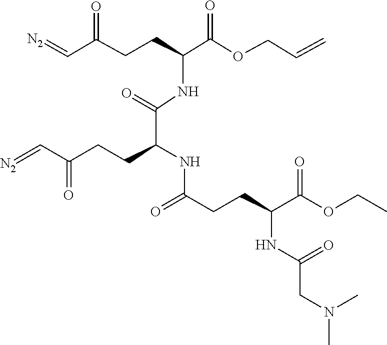

Compound 21a (75 mg, 0.144 mmol) in DCM (4 mL), 2,5-dioxopyrrolidin-1-yl dimethylglycinate (32 mg, 0.158 mmol), product 24a (35 mg, 40%) in a form of yellow solid.

¹H NMR (CDCl₃): 1.28 (t, J=7.1 Hz, 3H), 1.87-2.09 (m, 3H), 2.11-2.21 (m, 1H), 2.22-2.31 (m, 4H), 2.33 (s, 6H), 2.36-2.65 (m, 4H), 2.83-3.11 (m, 2H), 4.20 (qd, J=7.1, 1.0 Hz, 2H), 4.42 (q, J=7.0 Hz, 1H), 4.54 (td, J=8.2, 4.7 Hz, 1H), 4.57-4.66 (m, 3H), 5.26 (dq, J=10.4, 1.2 Hz, 1H), 5.29-5.44 (m, 3H), 5.90 (ddt, J=17.1, 10.4, 5.8 Hz, 1H), 7.05 (d, J=6.7 Hz, 1H), 7.45 (d, J=7.6 Hz, 1H), 7.73 (d, J=8.8 Hz, 1H). ESI-MS: 607.3 ([M+H]⁺).

### Allyl (6S,11S,14S)-6-(tert-butoxycarbonyl)-11,14-bis(4-diazo-3-oxobutyl)-2-methyl-4,9,12-trioxo-2,5,10,13-tetraazapentadecan-15-oate (24b)

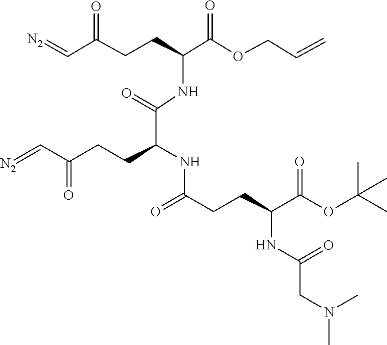

Compound 21b (74 mg, 0.135 mmol) in DCM (3 mL), 2,5-dioxopyrrolidin-1-yl dimethylglycinate (30 mg, 0.148 mmol), product 24b (40 mg, 47%) in a form of yellow solid.

¹H NMR (CDCl₃):1.47 (s, 9H), 1.82-1.92 (m, 1H), 1.95-2.08 (m, 2H), 2.11-2.20 (m, 1H), 2.21-2.31 (m, 4H), 2.33 (s, 6H), 2.36-2.65 (m, 4H), 2.87-3.08 (m, 2H), 4.42 (q, J=6.9 Hz, 1H), 4.49 (td, J=9.2, 3.9 Hz, 1H), 4.55 (td, J=8.1, 4.6 Hz, 1H), 4.62 (dq, J=5.8, 1.5 Hz, 2H), 5.26 (dq, J=10.4, 1.2 Hz, 1H), 5.29-5.44 (m, 3H), 5.90 (ddt, J=17.1, 10.4, 5.8 Hz, 1H), 7.13 (d, J=6.9 Hz, 1H), 7.44 (d, J=7.8 Hz, 1H), 7.72 (d, J=8.7 Hz, 1H). ESI-MS: 635.4 ([M+H]⁺).

### Ethyl (6S,11S,14S)-6-((allyloxy)carbonyl)-11,14-bis(4-diazo-3-oxobutyl)-2-methyl-4,9,12-trioxo-2,5,10,13-tetraazapentadecan-15-oate (24c)

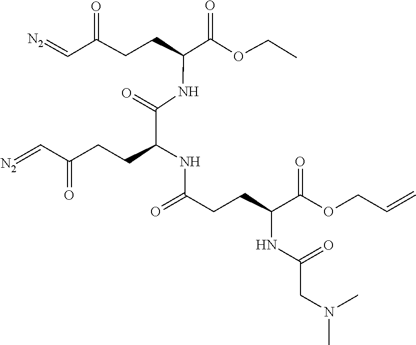

Compound 21c (150 mg, 0.288 mmol) in DCM (4 mL), 2,5-dioxopyrrolidin-1-yl dimethylglycinate (63 mg, 0.317 mmol), product 24c (58 mg, 64%) in a form of yellow solid.

¹H NMR (CDCl₃): 1.27 (t, J=7.1 Hz, 3H), 1.89-2.07 (m, 3H), 2.10-2.26 (m, 3H), 2.27-2.31 (m, 2H), 2.33 (s, 6H), 2.37-2.64 (m, 4H), 2.90-3.09 (m, 2H), 4.19 (q, J=7.2 Hz, 2H), 4.42 (q, J=7.1 Hz, 1H), 4.49-4.53 (m, 1H), 4.59-4.69 (m, 3H), 5.24-5.37 (m, 2H), 5.36-5.45 (m, 1H), 5.85-5.96 (m, 2H), 7.02 (d, J=7.3 Hz, 1H), 7.38 (d, J=7.8 Hz, 1H), 7.74 (d, J=9.0 Hz, 1H). ESI-MS: 607.3 ([M+H]⁺).

### tert-Butyl (6S,11S,14S)-6-((allyloxy)carbonyl)-11,14-bis(4-diazo-3-oxobutyl)-2-methyl-4,9,12-trioxo-2,5,10,13-tetraazapentadecan-15-oate (24d)

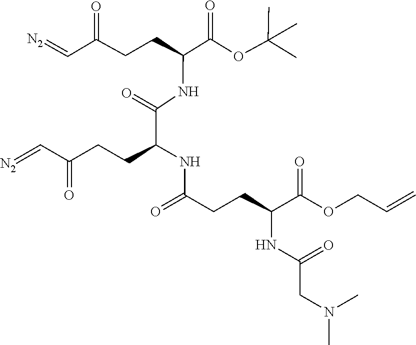

Compound 21d (115 mg, 0.209 mmol) in DCM (5 mL), 2,5-dioxopyrrolidin-1-yl dimethylglycinate (46 mg, 0.230 mmol), product 24d (46 mg, 35%) in a form of yellow amorphous compound.

¹H NMR (CDCl₃): 1.46 (s, 9H), 1.89-2.05 (m, 3H), 2.11-2.29 (m, 5H), 2.33 (s, 6H), 2.35-2.47 (m, 2H), 2.48-2.68 (m, 2H), 2.89-3.10 (m, 2H), 4.35-4.43 (m, 2H), 4.60-4.69 (m, 3H), 5.27 (dq, J=10.4, 1.2 Hz, 1H), 5.31-5.44 (m, 3H), 5.91 (ddt, J=16.9, 10.4, 5.9 Hz, 1H), 7.00 (d, J=8.1 Hz, 1H), 7.27 (d, J=2.5 Hz, 1H), 7.70-7.78 (m, 1H). ESI-MS: 635.4 ([M+H]⁺).

### Sodium (6S,11S,14S)-11,14-bis(4-diazo-3-oxobutyl)-6-(ethoxycarbonyl)-2-methyl-4,9,12-trioxo-2,5,10,13-tetraazapentadecan-15-oate (P11)

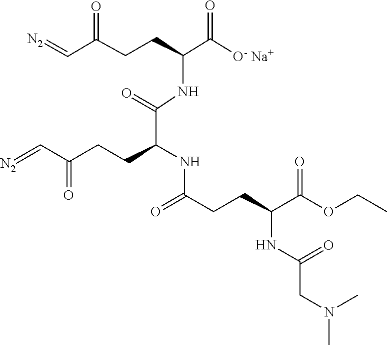

Compound 24a (27 mg, 0.045 mmol, 1 equiv) was dissolved in anhydrous DCM (1 mL) and phenylsilane (11 μL, 0.089 mmol, 2 equiv) and Pd(PPh₃)₄ (2.6 mg, 2.25 μmol, 0.05 equiv) were subsequently added. The reaction mixture was stirred for 50 min at rt and then concentrated in vacuo. The residue was purified by preparative HPLC (grad. 0-50% MeCN/H₂O in 50 min) to afford the product P11 (19 mg, 73%) as a yellow solid.

¹H NMR (D₂O): 1.23 (t, J=7.3 Hz, 3H), 1.89-2.04 (m, 3H), 2.08-2.13 (m, 2H), 2.14-2.22 (m, 1H), 2.33-2.47 (m, 6H), 2.48 (s, 6H), 3.38-3.45 (m, 2H), 4.12 (dd, J=8.3, 4.7 Hz, 1H), 4.19 (q, J=7.1 Hz, 2H), 4.24-4.32 (m, 1H), 4.38 (dd, J=9.1, 5.1 Hz, 1H), 5.82 (s, 1H), 5.86 (s, 1H). ESI-MS: 567.3 ([M+H]⁺).

### Sodium (6S,11S,14S)-6-carboxylato-11,14-bis(4-diazo-3-oxobutyl)-2-methyl-4,9,12-trioxo-2,5,10,13-tetraazapentadecan-15-oate (P12)

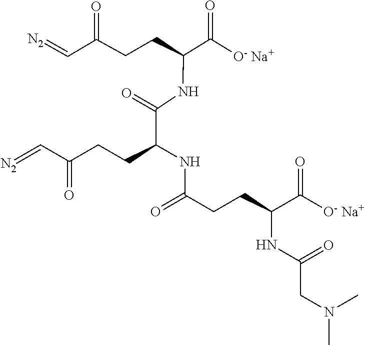

Compound 24c (23 mg, 0.041 mol, 1 equiv) was dissolved in MeOH (250 μL) and 1M NaOH (165 μL, 0.165 mmol, 4 equiv) was added. The reaction mixture was stirred for 30 min at rt and then concentrated in vacuo. The residue was purified by preparative HPLC (grad. 0-40% MeCN/H₂O in 50 min) to afford the product P12 (16 mg, 73%) as a yellow solid.

¹H NMR (D₂O): 1.85-1.99 (m, 3H), 2.05-2.15 (m, 3H), 2.30-2.41 (m, 4H), 2.44-2.49 (m, 2H), 2.90 (s, 6H), 3.98 (s, 2H), 4.10-4.15 (m, 2H), 4.27 (dd, J=8.5, 5.8 Hz, 1H). ESI-MS: 539.3 ([M+H]⁺).

### N⁵—((S)-6-diazo-1-(((S)-6-diazo-1-ethoxy-1,5-dioxohexan-2-yl)amino)-1,5-dioxohexan-2-yl)-N²-(dimethylglycyl)-L-glutamine (P13)

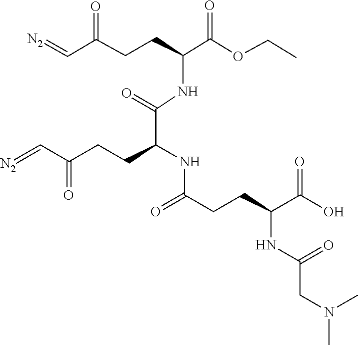

Compound 24c (30 mg, 0.049 mmol, 1 equiv) was dissolved in anhydrous DCM (1 mL) and phenylsilane (12 μL, 0.099 mmol, 2 equiv) and Pd(PPh₃)₄ (2.9 mg, 2.47 μmol, 0.05 equiv) were subsequently added. The reaction mixture was stirred for 1 h at rt and then concentrated in vacuo. The residue was purified by preparative HPLC (grad. 0-60% MeCN/H₂O in 50 min) to afford the product P13 (19 mg, 68%) as a colorless solid.

¹H NMR (D₂O): 1.22 (t, J=7.1 Hz, 3H), 1.86-2.01 (m, 3H), 2.03-2.12 (m, 2H), 2.14-2.24 (m, 2H), 2.30-2.36 (m, 2H), 2.46 (q, J=7.0 Hz, 3H), 2.69 (s, 6H), 3.69 (s, 2H), 4.12-4.20 (m, 3H), 4.22-4.30 (m, 1H), 4.31-4.38 (m, 1H). ESI-MS: 567.3 ([M+H]⁺).

### Sodium (6S,11S,14S)-6-(tert-butoxycarbonyl)-11,14-bis(4-diazo-3-oxobutyl)-2-methyl-4,9,12-trioxo-2,5,10,13-tetraazapentadecan-15-oate (P14)

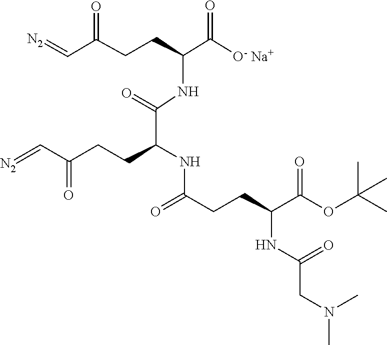

Compound 24b (36 mg, 0.057 mmol, 1 equiv) was dissolved in MeOH (400 μL) and 1M NaOH (170 μL, 0.170 mmol, 3 equiv) was added. The solution was stirred for 30 min at rt.

The volatiles were evaporated and the residue was purified by preparative HPLC (grad. 0-60% MeCN/H₂O in 50 min) to afford product P14 (21 mg, 62%) as a colorless solid.

¹H NMR (D₂O): 1.43 (s, 9H), 1.84-2.01 (m, 3H), 2.03-2.20 (m, 3H), 2.34-2.39 (m, 4H), 2.40 (s, 6H), 2.43-2.48 (m, 2H), 3.25-3.37 (m, 2H), 4.12 (dd, J=8.8, 4.9 Hz, 1H), 4.24 (dd, J=9.1, 5.2 Hz, 1H), 4.26-4.31 (m, 1H). ESI-MS: 595.3 ([M+H]⁺).

### Sodium N⁵—((S)-1-(((S)-1-(tert-butoxy)-6-diazo-1,5-dioxohexan-2-yl)amino)-6-diazo-1,5-dioxohexan-2-yl)-N²-(dimethylglycyl)-L-glutaminate (P15)

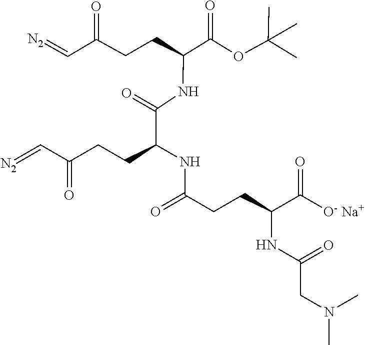

Compound 24d (42 mg, 0.066 mmol, 1 equiv) was dissolved in MeOH (300 μL) and 1M NaOH (200 μL, 0.199 mmol, 3 equiv) was added. The solution was stirred for 30 min at rt. The volatiles were evaporated and the residue was purified by preparative HPLC (grad. 0-50% MeCN/H₂O in 50 min) to afford product P15 (18 mg, 46%) as a colorless solid.

¹H NMR (D₂O): 1.42 (s, 9H), 1.84-2.01 (m, 3H), 2.02-2.21 (m, 3H), 2.28-2.37 (m, 2H), 2.38-2.51 (m, 4H), 2.61 (s, 6H), 4.09-4.18 (m, 1H), 4.19-4.23 (m, 1H), 4.23-4.32 (m, 1H). ESI-MS: 595.3 ([M+H]⁺).

### Allyl (5S,8S,13S,16S)-13,16-bis(4-diazo-3-oxobutyl)-8-(ethoxycarbonyl)-1-(9H-fluoren-9-yl)-5-isobutyl-3,6,11,14-tetraoxo-2-oxa-4,7,12,15-tetraazaheptadecan-17-oate (25)

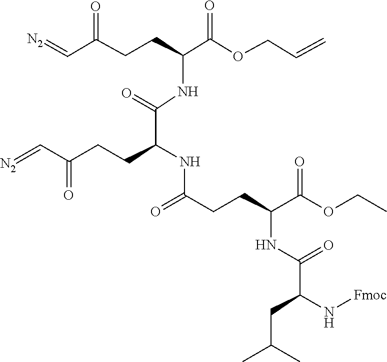

Compound 21a (185 mg, 0.355 mmol, 1 equiv) and HATU (155 mg, 0.408 mmol, 1.15 equiv) were suspended in anhydrous DCM (9 mL), cooled down to 0° C. and DIPEA (185 μL, 1.06 mmol, 3 equiv) was added. The mixture was stirred for 30 min at 0° C. and for 2 h at rt. The volatiles were evaporated in vacuo and the residue was purified by chromatography on silica (DCM/MeOH, 20:1) to afford product 25 (300 mg, 99%) as a yellow amorphous compound.

¹H NMR (DMSO-d₆): 0.88 (dd, J=15.9, 6.6 Hz, 6H), 1.16 (t, J=7.1 Hz, 3H), 1.40-1.50 (m, 2H), 1.60-1.74 (m, 2H), 1.79-1.89 (m, 3H), 1.93-2.06 (m, 2H), 2.15-2.44 (m, 6H), 4.07 (tq, J=11.5, 6.0 Hz, 3H), 4.16-4.35 (m, 6H), 4.50-4.59 (m, 2H), 5.16-5.23 (m, 1H), 5.29 (dq, J=17.2, 1.6 Hz, 1H), 5.87 (ddt, J=17.0, 10.7, 5.4 Hz, 1H), 5.99-6.09 (m, 2H), 7.31 (td, J=7.4, 2.9 Hz, 2H), 7.41 (t, J=7.4 Hz, 2H), 7.49 (d, J=8.2 Hz, 1H), 7.72 (t, J=7.9 Hz, 2H), 7.86-7.92 (m, 3H), 8.31 (d, J=7.5 Hz, 1H), 8.40 (d, J=7.4 Hz, 1H). ESI-MS: 879.4 ([M+Na]⁺).

### Allyl (S)-2-((S)-2-((S)-4-((S)-2-amino-4-methylpentanamido)-5-ethoxy-5-oxopentanamido)-6-diazo-5-oxohexanamido)-6-diazo-5-oxohexanoate (26)

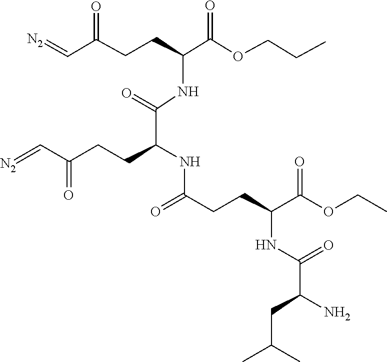

Compound 25 (300 mg, 0.350 mmol, 1 equiv) was dissolved in anhydrous DCM (3 mL) and DMF (0.5 mL) and diethylamine (724 μL, 7.00 mmol, 20 equiv) was added. The mixture was stirred for 1 h at rt. The volatiles were evaporated in vacuo. The residue was purified by chromatography on silica (DCM/MeOH, 10:1) and repurified by preparative HPLC (grad. 0-65% MeCN/H₂O in 50 min) to afford the desired product 26 (129 mg, 57%) as a yellow solid.

¹H NMR (CDCl₃): 0.97 (dd, J=15.8, 5.9 Hz, 6H), 1.28 (t, J=7.1 Hz, 3H), 1.65-1.87 (m, 3H), 1.88-2.15 (m, 4H), 2.16-2.30 (m, 2H), 2.35 (t, J=6.7 Hz, 2H), 2.40-2.69 (m, 6H), 4.09-4.16 (m, 1H), 4.17-4.25 (m, 2H), 4.40 (q, J=6.7 Hz, 1H), 4.52 (q, J=7.9 Hz, 1H), 4.56-4.67 (m, 3H), 5.23-5.28 (m, 1H), 5.31-5.37 (m, 1H), 5.47 (s, 1H), 5.51 (s, 1H), 5.90 (ddt, J=16.3, 10.6, 5.8 Hz, 1H), 7.17 (d, J=8.1 Hz, 1H), 7.39 (d, J=6.0 Hz, 1H), 7.72 (d, J=7.2 Hz, 1H). ESI-MS: 635.4 ([M+H]⁺).

### Sodium (S)-2-((S)-2-((S)-4-((S)-2-amino-4-methylpentanamido)-4-carboxylatobutanamido)-6-diazo-5-oxohexanamido)-6-diazo-5-oxohexanoate (P16)

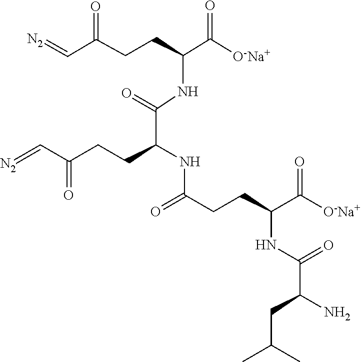

Compound 26 (22.5 mg, 0.035 mmol, 1 equiv) was dissolved in MeOH (250 μL) and 1M NaOH (215 μL, 0.213 mmol, 6 equiv) was added. The mixture was stirred for 45 min at rt. The volatiles were evaporated in vacuo and the residue was purified by preparative HPLC (grad. 0-50% MeCN/H₂O in 50 min) to afford product P16 (12 mg, 55%) as yellow solid.

¹H NMR (D₂O): 0.93 (dd, J=11.0, 6.1 Hz, 6H), 1.63-1.76 (m, 3H), 1.83-2.00 (m, 3H), 2.04-2.14 (m, 3H), 2.30-2.41 (m, 4H), 2.46 (t, J=7.4 Hz, 2H), 3.95 (t, J=6.8 Hz, 1H), 4.09-4.16 (m, 2H), 4.27 (dd, J=8.6, 5.7 Hz, 1H), 5.82 (s, 1H), 5.86 (s, 1H). ESI-MS: 567.3 ([M+H]⁺).

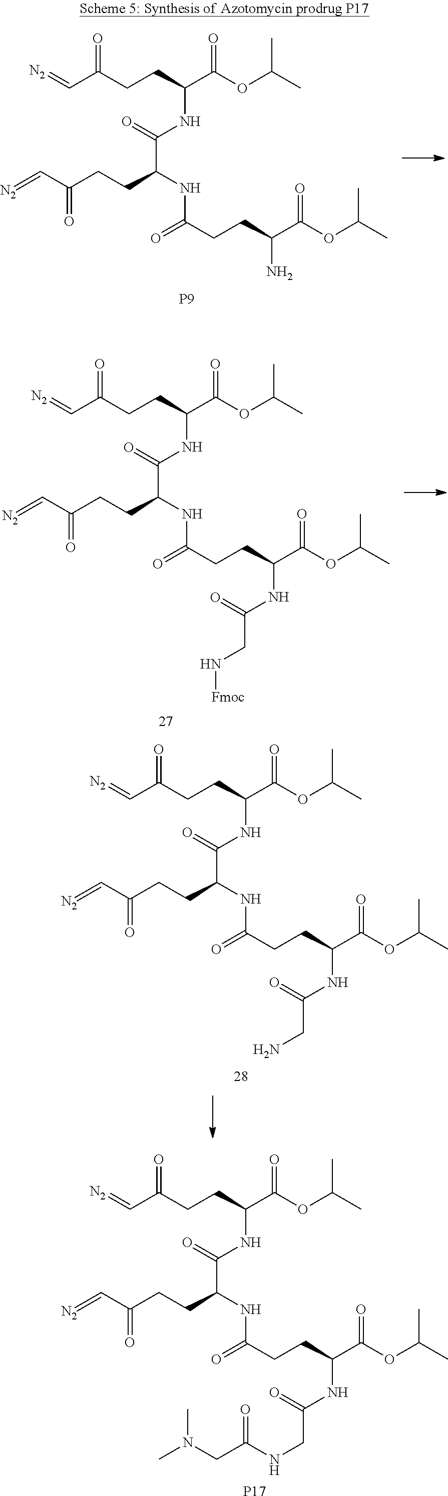

### Isopropyl (8S,13S,16S)-13,16-bis(4-diazo-3-oxobutyl)-1-(9H-fluoren-9-yl)-8-(isopropoxycarbonyl)-3,6,11,14-tetraoxo-2-oxa-4,7,12,15-tetraazaheptadecan-17-oate (27)

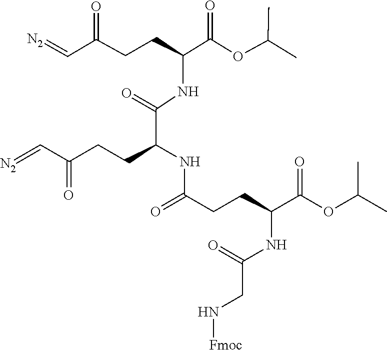

Fmoc-Gly-OH (24 mg, 0.082 mmol, 1.1 equiv) and HATU (33 mg, 0.086 mmol, 1.15 equiv) were dissolved in anhydrous DCM (3 mL) and DIPEA (39 μL, 0.223 mmol, 3 equiv) was added, followed by solution of compound P9 (40 mg, 0.074 mmol, 1 equiv) in DCM (2 mL). The mixture was Fmoc stirred at rt for 2 h, then diluted with DCM (50 mL) and washed with sat. NaHCO₃ (30 mL) and brine (30 mL). The organic layer was dried over anhydrous MgSO₄ and concentrated in vacuo. The residue was purified by chromatography on silica (DCM/MeOH, 20:1) to afford the product 27 (52 mg, 82%) as a yellow solid.

¹H NMR (CDCl₃): 1.21-1.29 (m, 12H), 1.93-2.08 (m, 3H), 2.08-2.35 (m, 4H), 2.35-2.62 (m, 5H), 3.85-4.01 (m, 2H), 4.22-4.27 (m, 1H), 4.38-4.44 (m, 3H), 4.43-4.49 (m, 1H), 4.50-4.56 (m, 1H), 5.00-5.09 (m, 2H), 5.25-5.39 (m, 2H), 5.70 (t, J=5.1 Hz, 1H), 6.92 (d, J=6.3 Hz, 1H), 7.16 (d, J=6.8 Hz, 1H), 7.29-7.43 (m, 5H), 7.62 (d, J=7.5 Hz, 2H), 7.76 (d, J=7.6 Hz, 2H). ESI-MS: 839.3 ([M+Na]).

### Isopropyl (S)-2-((S)-2-((S)-4-(2-aminoacetamido)-5-isopropoxy-5-oxopentanamido)-6-diazo-5-oxohexanamido)-6-diazo-5-oxohexanoate (28)

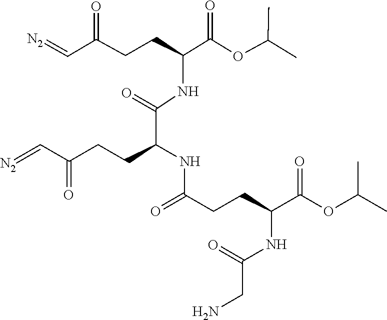

Compound 27 (52 mg, 0.064 mmol, 1 equiv) was dissolved in anhydrous DCM (1.1 mL) and diethylamine (132 μL, 1.27 mmol, 20 equiv) was added. The mixture was stirred at rt for 3 h and then concentrated in vacuo. The residue was purified by chromatography on silica (DCM/MeOH, 5:1) to afford product 28 (24 mg, 63%) as a yellow solid.

¹H NMR (CDCl₃): 1.14-1.30 (m, 12H), 1.92-2.06 (m, 5H), 2.11-2.30 (m, 3H), 2.30-2.35 (m, 2H), 2.36-2.49 (m, 2H), 2.49-2.64 (m, 2H), 3.43 (s, 2H), 4.42 (q, J=6.6 Hz, 1H), 4.48 (td, J=7.7, 4.2 Hz, 1H), 4.56 (td, J=9.0, 4.2 Hz, 1H), 4.98-5.09 (m, 2H), 5.32-5.42 (m, 2H), 7.11 (d, J=6.6 Hz, 1H), 7.37 (d, J=9.3 Hz, 1H), 7.90 (d, J=8.5 Hz, 1H).

ESI-MS: 595.3 ([M+H]⁺).

### Isopropyl (9S,14S,17S)-14,17-bis(4-diazo-3-oxobutyl)-9-(isopropoxycarbonyl)-2-methyl-4,7,12,15-tetraoxo-2,5,8,13,16-pentaazaoctadecan-18-oate (P17)

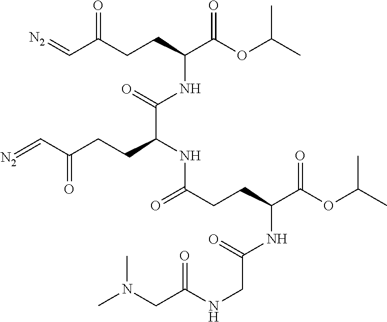

Compound 28 (23 mg, 0.039 mmol, 1 equiv) and 2,5-dioxopyrrolidin-1-yl dimethylglycinate (9.3 mg, 0.046 mmol, 1.2 equiv) were dissolved in anhydrous DCM (1 mL) and the solution was stirred for 3 h at rt. The mixture was diluted with DCM (30 mL) and washed with sat. NaHCO₃ (15 mL) and brine (15 mL). The organic layer was dried over anhydrous MgSO₄ and concentrated in vacuo. The residue was purified by chromatography on silica (DCM/MeOH, 5:1) to afford product P17 (17 mg, 65%) as a yellow solid.

¹H NMR (CDCl₃): 1.24 (d, J=6.1 Hz, 12H), 1.92-2.16 (m, 4H), 2.17-2.31 (m, 4H), 2.33 (s, 6H), 2.36-2.60 (m, 4H), 2.94-3.11 (m, 2H), 3.91-4.06 (m, 2H), 4.37-4.54 (m, 3H), 4.97-5.06 (m, 2H), 5.37-5.46 (m, 2H), 7.09 (d, J=6.1 Hz, 1H), 7.30 (d, J=7.3 Hz, 1H), 7.51 (d, J=7.6 Hz, 1H), 7.76-7.83 (m, 1H). ESI-MS: 680.3 ([M+H]⁺).

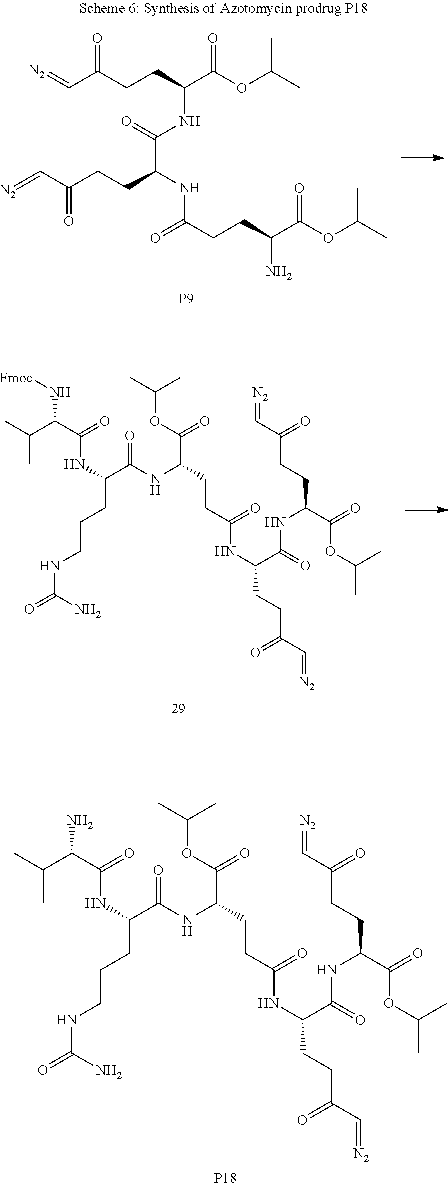

### Isopropyl (5S,8S,11S,16S,19S)-16,19-bis(4-diazo-3-oxobutyl)-1-(9H-fluoren-9-yl)-11-(isopropoxycarbonyl)-5-isopropyl-3,6,9,14,17-pentaoxo-8-(3-ureidopropyl)-2-oxa-4,7,10,15,18-pentaazaicosan-20-oate (29)

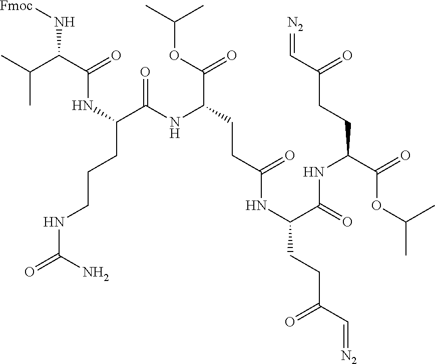

Fmoc-Val-Cit-OH (41 mg, 0.818 mmol, 1.1 equiv) and HATU (33 mg, 0.086 mmol, 1.15 equiv) were dissolved in anhydrous DMF (2 mL) and DIPEA (39 μL, 0.223 mmol, 3 equiv) was added. The mixture was cooled down to 0° C. and the solution of compound P9 (40 mg, 0.074 mmol, 1 equiv) was added dropwise. The mixture was stirred at rt for 19 h and then concentrated in vacuo. The crude product 29 was used to the further reaction without any purification and characterization.

### Isopropyl (6S,9S,14S,17S)-1-amino-6-((S)-2-amino-3-methylbutanamido)-14,17-bis(4-diazo-3-oxobutyl)-9-(isopropoxycarbonyl)-1,7,12,15-tetraoxo-2,8,13,16-tetraazaoctadecan-18-oate (P18)

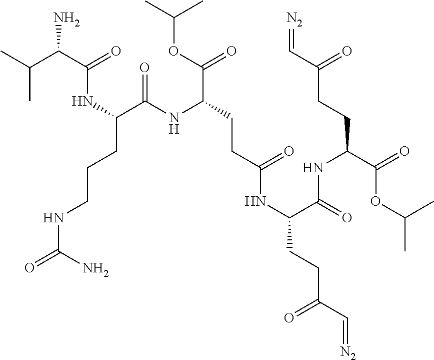

The crude mixture 29 was dissolved in anhydrous DCM (2 mL) and diethylamine (600 μL, 5.80 mmol, 78 equiv) was added. The mixture was stirred for 1 h at rt and then concentrated in vacuo. The residue was purified by chromatography on silica (DCM/MeOH, grad 7:1 to 2:1) to afford product P18 (41 mg, 54%) as a yellow solid. ESI-MS: 794.5 ([M+H]⁺)

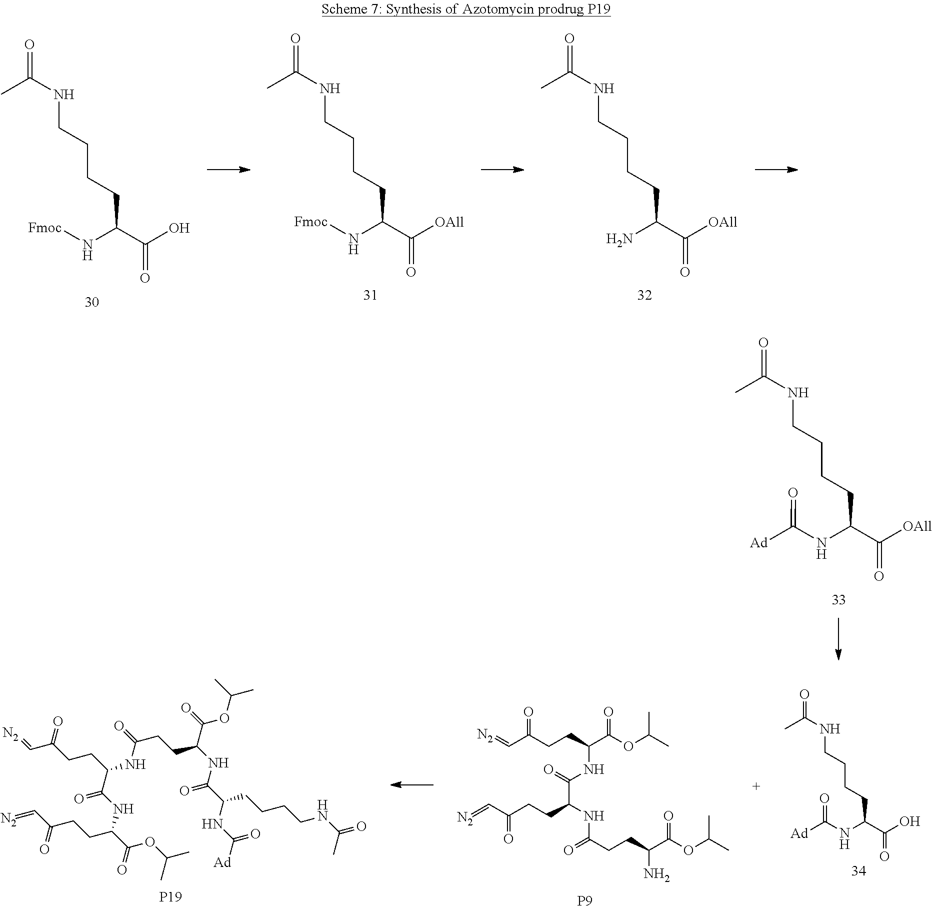

### (E)-Prop-1-en-1-yl N2-(((9H-fluoren-9-yl)methoxy)carbonyl)-N6-acetyl-L-lysinate (31)

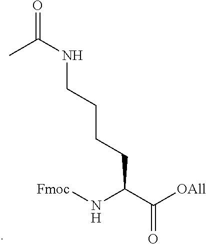

Fmoc-Lys(Ac)—OH (1.0 G, 2.44 mmol, 1 equiv) was dissolved in anhydrous DMF (15 mL) and K₂CO₃ (505 mg, 3.65 mmol, 1.5 equiv) was added, followed by allyl bromide (274 μL, 3.17 mmol, 1.3 equiv). The resulting mixture was stirred at rt for 4 h. The volatiles were evaporated, the residue was diluted with EtOAc (100 mL) and washed with dist. H₂O (100 mL) and brine (100 mL). The organic layer was dried over anhydrous MgSO₄ and concentrated in vacuo. The product 31 (1.05 G, 95%) was obtained as a colorless solid. ESI-MS: 451.3 ([M+H]⁺).

### (E)-Prop-1-en-1-yl N6-acetyl-L-lysinate (32)

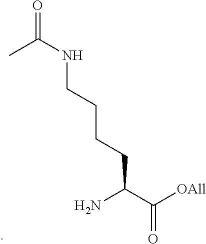

Compound 31 (1.05 G, 2.33 mmol, 1 equiv) was dissolved in anhydrous DCM (20 mL) and DMF (1 mL) and diethylamine (4.8 mL, 46.6 mmol, equiv) was added. The mixture was stirred at rt for 2 h and then concentrated in vacuo. The crude product 32 was used to the further reaction without any purification and characterization.

### (E)-Prop-1-en-1-yl N6-acetyl-N2-((3S,5S,7S)-adamantane-1-carbonyl)-L-lysinate (33)

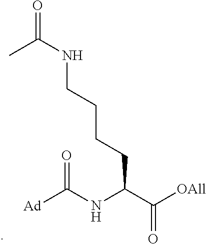

Adamantane carboxylic acid (420 mg, 2.33 mmol, 1 equiv) and HATU ANH (975 mg, 2.56 mmol, 1.1 equiv) were dissolved in anhydrous DMF (15 mL) and DIPEA (1.2 mL, 6.99 mmol, 3 equiv) was added, followed by the solution of compound 32 (532 mg, 2.33 mmol, 1 equiv) in anhydrous DMF (10 mL). The reaction mixture was stirred at rt for 1.5 h and then concentrated in vacuo. The residue was diluted with EtOAc (100 mL) and washed with sat. NaHCO₃ (100 mL), dist. H₂O (100 mL), 10% KHSO₄ (100 mL) and brine (100 mL). The organic layer was dried over anhydrous MgSO₄ and concentrated in vacuo. The residue was purified by chromatography on silica (DCM/MeOH, 20:1) to afford product 33 (700 mg, 77%) as a yellow solid.

¹H NMR (CDCl₃): 1.35 (p, J=7.6 Hz, 2H), 1.49-1.62 (m, 2H), 1.63-1.79 (m, 8H), 1.82-1.90 (m, 6H), 1.98 (s, 3H), 2.02-2.07 (m, 3H), 3.14-3.32 (m, J=6.9 Hz, 2H), 4.54-4.69 (m, 3H), 5.22-5.38 (m, 2H), 5.79-5.85 (m, 1H), 5.90 (ddt, J=16.6, 11.4, 5.8 Hz, 1H), 6.22 (d, J=7.7 Hz, 1H). ESI-MS: 391.3 ([M+H]⁺).

### N6-Acetyl-N2-((3S,5S,7S)-adamantane-1-carbonyl)-L-lysine (34)

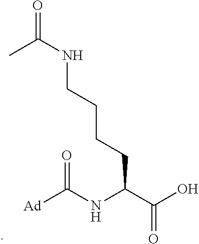

Compound 33 (668 mg, 1.71 mmol, 1 equiv) was dissolved in anhydrous DCM (18 mL) and PhSiH₃ (422 μL, 3.42 mmol, 2 equiv), followed by Pd(PPh₃)₄ (40 mg, 0.034 mmol, 0.02 equiv) was added. The reaction mixture was stirred at rt for 1.5 h. The volatiles were evaporated and the residue was purified by chromatography on silica (DCM/MeOH, grad. 10:1 to 5:1) to afford product 34 (480 mg, 80%) as a colorless solid.

¹H NMR (DMSO-d₆): 1.16-1.28 (m, 2H), 1.30-1.39 (m, 2H), 1.58-1.72 (m, 8H), 1.74-1.83 (m, 9H), 1.96 (s, 3H), 2.92-3.02 (m, 2H), 4.05-4.11 (m, 1H), 7.29 (d, J=6.8 Hz, 1H), 7.79 (t, J=5.0 Hz, 1H). ESI-MS: 349.2 ([M−H]⁻).

### Isopropyl (2S,5S,10S,13S)-13-((3S,5S,7S)-adamantane-1-carboxamido)-2,5-bis(4-diazo-3-oxobutyl)-10-(isopropoxycarbonyl)-4,7,12,19-tetraoxo-3,6,11,18-tetraazaicosanoate (P19)

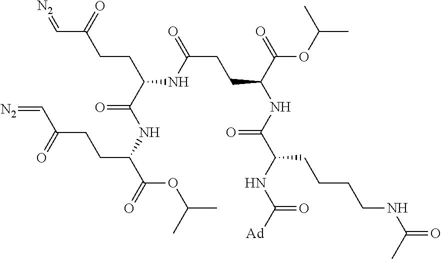

Compound 34 (34 mg, 0.063 mmol, 1 equiv) and HATU (28 mg, 0.073 mmol, 1.15 equiv) were dissolved in anhydrous DMF (1.5 mL) and cooled down to 0° C. DIPEA (33 μL, 0.190 mmol, 3 equiv) was added, followed by the solution of compound P9 (34 mg, 0.063 mmol, 1 equiv) in anhydrous DMF (1.5 mL). The reaction mixture was stirred at rt for 1.5 h and then concentrated in vacuo. The residue was purified by chromatography on silica (DCM/MeOH, 15:1) to afford product P19 (32 mg, 58%) as a yellow solid.

¹H NMR (CDCl₃): 1.20-1.28 (m, 12H), 1.58-1.64 (m, 5H), 1.66-1.78 (m, 6H), 1.84-1.90 (m, 6H), 1.98 (s, 3H), 2.00-2.07 (m, 3H), 2.10-2.62 (m, 9H), 3.18 (ddq, J=11.5, 7.4, 4.0 Hz, 2H), 3.22-3.28 (m, 2H), 3.68-3.77 (m, 2H), 4.31-4.58 (m, 4H), 4.96-5.09 (m, 2H), 5.33-5.53 (m, 2H), 6.30 (d, J=6.4 Hz, 1H), 7.12-7.21 (m, 1H), 7.47 (d, J=6.7 Hz, 1H), 7.70 (d, J=7.8 Hz, 1H), 8.02 (s, 1H). ESI-MS: 870.5 ([M+H]⁺)

### 1.3. Metabolic Stability

### 1.3.1 Metabolic Stability Methods

In vitro stability studies were done using CES1KO mouse plasma, human plasma, CES1-KO mouse liver, and human liver microsomes as previously described [1]. For tissue homogenate stability studies, washed tissue was diluted 10-fold in 0.1 M potassium phosphate buffer and homogenized using probe sonication. Crude homogenate was then aliquoted to 1 mL, and spiked with 10 mM stock of the analyte in DMSO to achieve a 10 μM final concentration. The plasma stability of the prodrugs was determined by spiking the analyte in 1 mL of plasma. The plasma and tissues were incubated in an orbital shaker at 37° C. Stability in human liver microsomes was assessed at a final prodrug concentration of 1 μM with 0.2 mg/mL protein for microsomes. All the stability studies were conducted at predetermined times (0 and 60 min), where 50 μL aliquots of the mixture in triplicate were removed and the reaction was quenched by addition of 5 times the volume of ice-cold methanol spiked with the internal standard (losartan: 0.5 μM). The samples were vortex-mixed for 30 s and centrifuged at 10,000 g for 10 min at 4° C. An amount of 50 μL of the supernatant was diluted with 50 μL of water and transferred to a 250 μL polypropylene vial sealed with a Teflon cap. Prodrug disappearance was monitored over time using liquid chromatography and mass spectrometry (LC-MS).

### 1.3.2 Bioanalysis—Metabolic Stability

Analyses were performed on a Dionex ultra high-performance LC system coupled with Q Exactive Focus orbitrap mass spectrometer (Thermo Fisher Scientific Inc., Waltham MA). Separation was achieved using Agilent Eclipse Plus column (100×2.1 mm i.d., maintained at 35° C.) packed with a 1.8 μm C18 stationary phase. The mobile phase consisted of 0.1% formic acid in water and 0.1% formic acid in acetonitrile, lumps were operated at a flow rate of 0.3 mL/min for 7 min using gradient elution. The mass spectrometer controlled by Xcalibur software 4.0.27.13 (Thermo Scientific) was operated with a HESI ion source in positive ionization mode. Metabolites were identified in the full-scan mode (from m/z 50 to 1600) by comparing t═O samples

TABLE 4
Metabolic Studies

| | Liver Stability | | Plasma Stability | |
|---|---|---|---|---|
| | (% Remaining at 60 min) | | (% Remaining at 60 min) | |
| | CES1^{-/-} | Human Liver | CES1^{-/-} | Human |
| Code | Mouse Liver | Microsomes | Mouse Plasma | Plasma |
| Azotomycin | 89.7 ± 0.88 | 23.7 ± 0.333 | 105 ± 7.64 | 74.3 ± 1.20 |
| P1 | 87.0 ± 8.51 | 105 ± 0.859 | 101 ± 3.38 | 104 ± 1.00 |
| P2 | 100 ± 1.67 | 85.3 ± 7.88 | 104 ± 2.31 | 110 ± 4.70 |
| P3 | 96.0 ± 4.36 | 105 ± 2.53 | 92.3 ± 0.882 | 106 ± 2.40 |
| P4 | 54.3 ± 1.45 | 107 ± 1.63 | 101 ± 4.67 | 100 ± 0.00 |
| P5 | 4.33 ± 0.882 | 95.4 ± 0.595 | 87.7 ± 8.51 | 97.3 ± 2.85 |
| P6 | 85.0 ± 5.86 | 98.5 ± 0.676 | 99.0 ± 3.51 | 105 ± 1.76 |
| P7 | 109 ± 10.0 | 95.3 ± 0.168 | 98.0 ± 12.5 | 98.7 ± 2.67 |
| P8 | 49.7 ± 0.333 | 95.8 ± 0.141 | 99.3 ± 1.45 | 109 ± 1.00 |
| P9 | 0.320 ± 0.026 | >100 | 84.3 ± 10.5 | 85.9 ± 3.63 |
| P10 | 90.4 ± 2.76 | 90.4 ± 4.10 | 104 ± 3.67 | 90.5 ± 6.35 |
| P11 | 96.3 ± 1.80 | 103 ± 4.97 | 91.8 ± 4.16 | 83.6 ± 1.76 |
| P12 | 111 ± 9.27 | 99.6 ± 3.50 | 95.6 ± 0.44 | 76.2 ± 7.25 |
| P13 | 1.55 ± 0.77 | 109 ± 2.22 | 107 ± 6.6 | 86.6 ± 2.69 |
| P14 | 82.4 ± 4.03 | 103 ± 2.31 | 111 ± 4.09 | 96.2 ± 3.21 |
| P15 | 99.9 ± 3.45 | 93.2 ± 4.29 | >100 | 104 ± 8.45 |
| P16 | 4.50 ± 1.58 | 91.0 ± 1.01 | 4.17 ± 0.58 | 87.4 ± 1.61 |
| P17 | 0.964 ± 0.031 | 98.0 ± 5.36 | 94.0 ± 2.47 | 93.7 ± 4.92 |
| P18 | 0.11 ± 0.03 | 84.9 ± 2.30 | 105 ± 5 | 105 ± 4.81 |
| P19 | 0.502 ± 0.103 | 74.1 ± 2.10 | 85.1 ± 0.64 | 97.0 ± 4.11 |

with t = 60 min samples.

[symbol]

[symbol]

CES1KO = Carboxylesterase 1 knock-out

### 1.4 Pharmacokinetics Screening of Prodrugs

### 1.4.1 Methods

The pharmacokinetic study in C57BL/6 CES1 mice was conducted according to protocols reviewed and approved by the Johns Hopkins Institutional Animal Care and Use Committee in compliance with the Association for Assessment and Accreditation of Laboratory Animal Care International and the Public Health Service Policy on the Humane Care and Use of Laboratory Animals (PHS Policy). Briefly, naïve male and female C57BL/6 CES1^{-/-} mice (weighing between 25 and 30 g) 6-8 weeks of age were used. The animals were maintained on a 12 h light-dark cycle with ad libitum access to food and water. Tumors were grown via subcutaneous (SC) injection of EL4 cells (1×10⁶ cells in 0.2 mL of phosphate-buffered saline) in one location on the flank of each mouse. When tumors grew to a mean volume of around 400 mm³, mice were used for pharmacokinetic study Prior to dosing, the interscapular region was wiped with alcohol gauze.

Compounds were dissolved in ethanol/Tween 80/saline (5:10:85 v/v/v) and were administered to nice as a, single SC dose of 1 mg/kg equivalent to Azotomycin. The mice were euthanized with carbon dioxide at the time-points indicated for pk analysis. Blood samples (˜0.8 mL) were collected in heparinized microtubes by cardiac puncture, and jejunum, as well as tumors were removed and flash-frozen on dry ice. Blood samples were centrifuged at a temperature of 4° C. at 3000 g for 10 ruin. All samples were kept chilled throughout processing. Plasma samples (˜300 μL) were collected in polypropylene tubes and stored at −80° C. until bioanalysis. Flash-frozen jejunum, and tumor samples were also stored at −80° C. until bioanalysis.

For quantifying intact analytes in the pharmacokinetic samples, plasma samples (25 μL) were processed using a single step protein precipitation method by addition of 125 μL of methanol containing internal standard (losartan: 0.5 μM), followed by vortex-mixing for 30 s and then centrifugation at 16,000 g for 5 min at 4° C. Jejunum and tumor tissues were diluted 1:5 w/v with methanol containing losartan (0.5 μM) and homogenized, followed by vortex-mixing and centrifugation at 16,000 g for 5 min at 4° C. A Supernatants were transferred to autosampler vials. Then, 2 μL of the sample was injected into the LCMS system.

The samples were subjected to dabsyl chloride derivatization and analyzed by LC-MS/MS, as described in Tenora et al., 2019. DON was extracted from plasma samples by protein precipitation using methanol. Briefly, standards, QCs, and samples (50 μL) were mixed with 250 μL of methanol containing 10 μM glutamate-d5 (internal standard) in low-retention microcentrifuge tubes. Jejunum and tumor samples were weighed. Five microliters of methanol containing 10 μM glutamate-d5 was added per milligram of the tissue sample and samples were mechanically homogenized. For plasma, jejunum, and tumor, the mixture was vortex-mixed and centrifuged at 16,000 g for 5 min at 4° C. The supernatant (100 μL) was transferred to a new tube and dried under vacuum at 45° C. for 1 h. The samples were subjected to dabsyl chloride derivatization and analyzed by LC-MS/MS, as described in Tenora et al., 2019.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Tenora, L. et al. Tumor-Targeted Delivery of 6-Diazo-5-oxo-1-norleucine (DON) Using Substituted Acetylated Lysine Prodrugs, J. Med. Chem. 2019, 62, 3524-3538.
Yokoyama, Y. et al., Sirpiglenastat (DRP-104) Induces Antitumor Efficacy through Direct, Broad Antagonism of Glutamine Metabolism and Stimulation of the Innate and Adaptive Immune Systems, Mol. Cancer Ther., 2022, 21(10), 1561-1572.
Rais, R. et al., Discovery of DRP-104, a tumor-targeted metabolic inhibitor prodrug, Sci. Adv., 2022, 8, eabq5925.
Leone R. D., et al., Glutamine blockade induces divergent metabolic programs to overcome tumor immune evasion, Science, 2019, 366, 1013-1021.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A compound of formula (I): 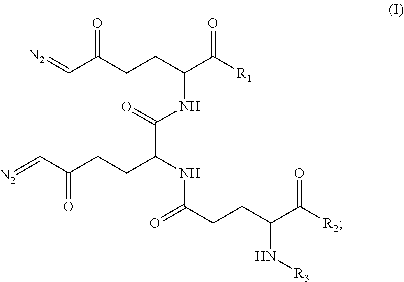
wherein:
R₁ and R₂ are each independently selected from —OR₄, —NR₅R₆, —O⁻M⁺, and —O—(CH₂CH₂O)ₙ—R₇, wherein:
n is an integer selected from 1, 2, 3, and 4;
R₄ is selected from H and C₁-C₄ substituted or unsubstituted branched or unbranched alkyl;
R₅ and R₆ are each independently H, C₁-C₄ substituted or unsubstituted branched or unbranched alkyl, -AA-COOR₇, wherein R₇ is C₁-C₄ substituted or unsubstituted branched or unbranched alkyl, and AA is an amino acid;
M⁺ is a metal cation;
R₃ is selected from H, —(C═O)—R₈, —C(═O)—CH₂—(NH—C(═O)—CH₂—NR₉R₁₀)—CH₂—R₁₁, —C(═O)—CH—(NH—C(═O)—CH(NH₂)—CH—(CH₃)₂)—((CH₂)₃NH—C(═O)NH₂, —C(═O)—CH((CH₂)₄—NH—C(═O)—CH₃)(NH—C(═O)-adamantane), —C(═O)-AA-R₁₂, acetyl (Ac), dimethylglycine, dimethylglycyl (DMG), L-Lys(Ac), D-Val-L-Leu-L-Lys(Ac), D-Val-L-Leu-L-Lys(H), D-Val-L-Leu-L-Lys(DMG), L-Val-L-Cit, para-aminobenzoic acid (PABA), L-Cit-PABA, L-Val-L-Cit-PABA, Val-Cit-OH, dimethylglycyl-L-tryptophan, acetoxymethyl pivalate, 1-acetoxyethyl pivalate, acetoxy(phenyl)methyl pivalate, and 2-acetoxypropan-2-yl pivalate; wherein AA is an amino acid, wherein the amino acid of R₃, R₅, and R₆ can be selected from an amino acid or amino acid-related substituent group listed in Table 2 or Table 3, and combinations thereof;
wherein:
R₈ is selected from C₁-C₄ substituted or unsubstituted branched or unbranched alkyl and —CH₂—NR₁₄R₁₅;
R₉, R₁₀, R₁₄, and R₁₅ are each independently selected from H and C₁-C₄ substituted or unsubstituted branched or unbranched alkyl;
R₁₁ is selected from substituted or unsubstituted aryl and substituted or unsubstituted heteroaryl;
R₁₂ is selected from H, —(C═O)—R₁₃, and —NH-dimethylglycyl;
R₁₃ is C₁-C₄ substituted or unsubstituted branched or unbranched alkyl;
provided that R₁ and R₂ cannot both be —OH if R₃ is H; and
stereoisomers and pharmaceutically acceptable salts thereof.

2. The compound of claim 1, wherein the compound of formula (I) is a compound of formula (Ia): 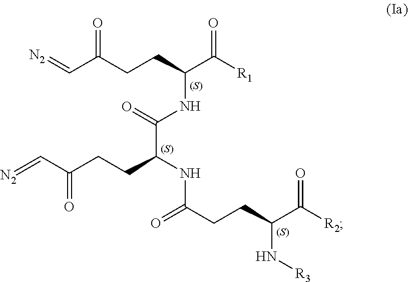
wherein:
R₁ and R₂ are each independently selected from —OR₄, —NR₅R₆, and —O—(CH₂CH₂O)ₙ—R₇, wherein:
n is an integer selected from 1, 2, 3, and 4;
R₄ is C₁-C₄ substituted or unsubstituted branched or unbranched alkyl;
R₅ and R₆ are each independently H, C₁-C₄ substituted or unsubstituted branched or unbranched alkyl, -AA-COOR₇, wherein R₇ is C₁-C₄ substituted or unsubstituted branched or unbranched alkyl, and AA is an amino acid;
R₃ is selected from H, —(C═O)—R₈, —C(═O)—CH(NR₉R₁₀)—R₇, —C(═O)—CH₂—(NH—C(═O)—CH₂—NR₉R₁₀)—CH₂—R₁₁, —C(═O)-AA-R₁₂, acetyl (Ac), dimethylglycine, dimethylglycyl (DMG), L-Lys(Ac), D-Val-L-Leu-L-Lys(Ac), D-Val-L-Leu-L-Lys(H), D-Val-L-Leu-L-Lys(DMG), L-Val-L-Cit, para-aminobenzoic acid (PABA), L-Cit-PABA, L-Val-L-Cit-PABA, dimethylglycyl-L-tryptophan, acetoxymethyl pivalate, 1-acetoxyethyl pivalate, acetoxy(phenyl)methyl pivalate, and 2-acetoxypropan-2-yl pivalate; wherein AA is an amino acid, wherein the amino acid of R₃, R₅, and R₆ can be selected from an amino acid or amino acid-related substituent group listed in Table 2 or Table 3, and combinations thereof;
wherein:
R₈ is selected from C₁-C₄ substituted or unsubstituted branched or unbranched alkyl and —CH₂—NR₁₄R₁₅;
R₉, R₁₀, R₁₄, and R₁₅ are each independently selected from H and C₁-C₄ substituted or unsubstituted branched or unbranched alkyl;
R₁₁ is selected from substituted or unsubstituted aryl and substituted or unsubstituted heteroaryl;
R₁₂ is selected from H, —(C═O)—R₁₃, and —NH-dimethylglycyl;
R₁₃ is C₁-C₄ substituted or unsubstituted branched or unbranched alkyl; and
stereoisomers and pharmaceutically acceptable salts thereof.

3. The compound of claim 1, wherein R₁ and R₂ are each independently selected from —OCH₂CH₃, —O—CH(CH₃)₂, —O—C(CH₃)₃, —NH₂, —NHCH₃, —NH-AA-COO—CH(CH₃)₂, —NH-AA-COO—C(CH₃)₃, —O—(CH₂CH₂O)ₙ—H, —O—(CH₂CH₂O)ₙ-AA-COO—CH(CH₃)₂, and —O—(CH₂CH₂O)ₙ-AA-COO—C(CH₃)₃.

4. The compound of claim 1, wherein R₃ is selected from H, acetyl (Ac), dimethylglycine, dimethylglycyl (DMG), L-Lys(Ac), D-Val-L-Leu-L-Lys(Ac), D-Val-L-Leu-L-Lys(H), D-Val-L-Leu-L-Lys(DMG), L-Val-L-Cit, para-aminobenzoic acid (PABA), L-Cit-PABA, L-Val-L-Cit-PABA, dimethylglycyl-L-tryptophan, acetoxymethyl pivalate, 1-acetoxyethyl pivalate, acetoxy(phenyl)methyl pivalate, and 2-acetoxypropan-2-yl pivalate, —C(═O)-AA-H, —C(═O)-AA-Ac, and —C(═O)-AA-NH-dimethylglycyl.

5. The compound of claim 1, wherein R₁ and R₂ are each —OR₄.

6. The compound of claim 5, wherein one or both of R₁ and R₂ are each selected from —OCH₂CH₃, —OCH(CH₃)₂, and —OC(CH₃)₃.

7. The compound of claim 1, wherein R₁ and R₂ are each —NR₅R₆.

8. The compound of claim 7, wherein R₁ and R₂ are each —NH₂.

9. The compound of claim 1, wherein R₃ is selected from H, —C(═O)—R₈, and —C(═O)—CH₂—(NH—C(═O)—CH₂—NR₉R₁₀)—CH₂—R₁₁.

10. The compound of claim 9, wherein R₃ is selected from H, —C(═O)—CH₃, —C(═O)—CH₂—N(CH₃)₂, and —C(═O)—CH₂—(NH—C(═O)—CH₂—N(CH₃)₂)—CH₂-(1H)indole.

11. The compound of claim 6, wherein the compound is selected from:

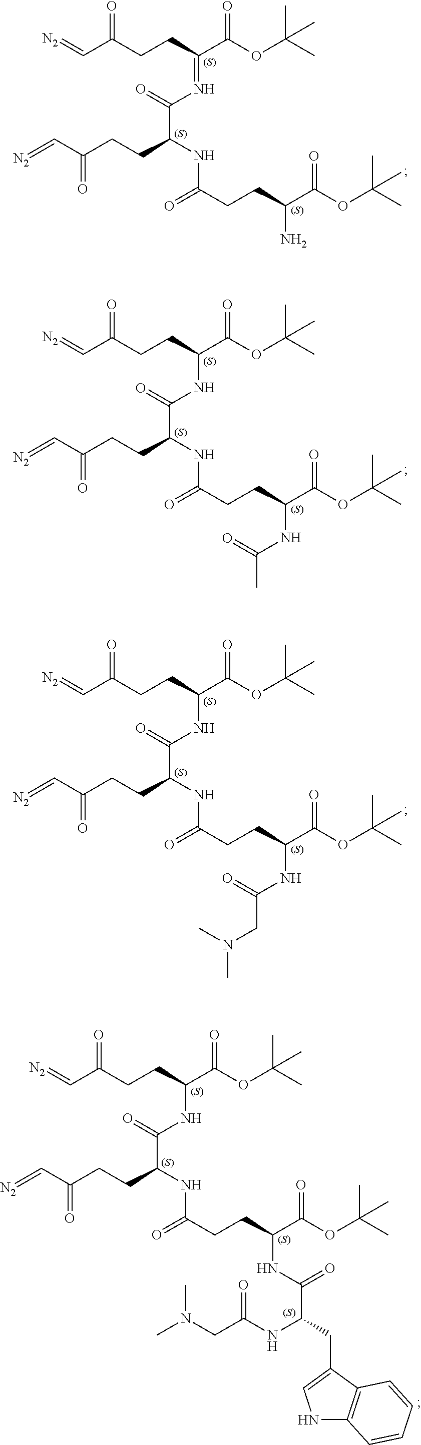

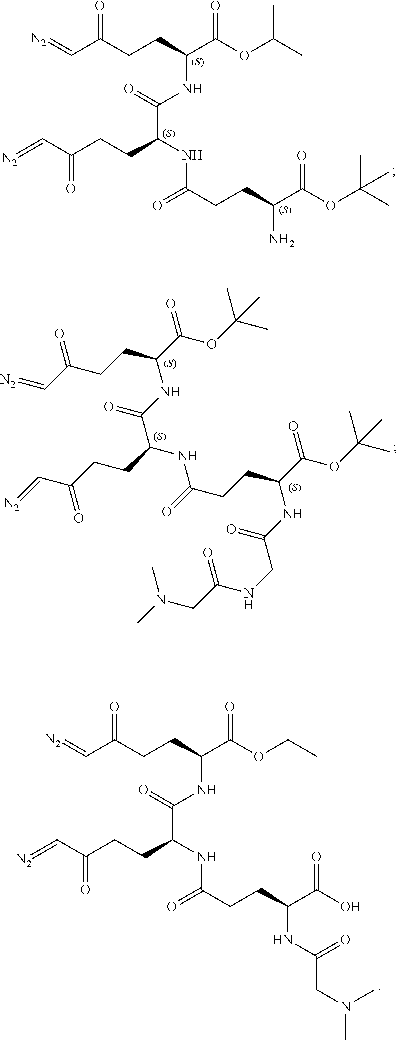

12. The compound of claim 11, wherein the compound of formula (I) is selected from:

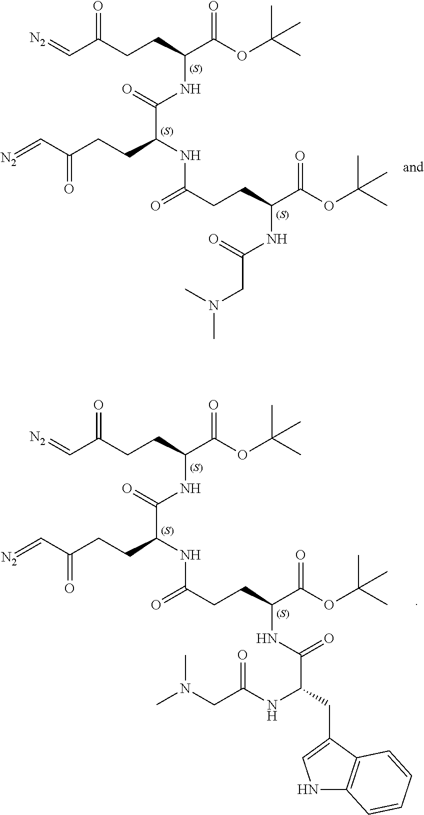

13. The compound of claim 12, wherein the compound of formula (I) is:

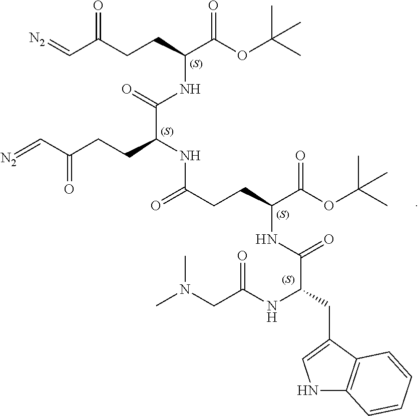

14. The compound of claim 8, wherein the compound is selected from:

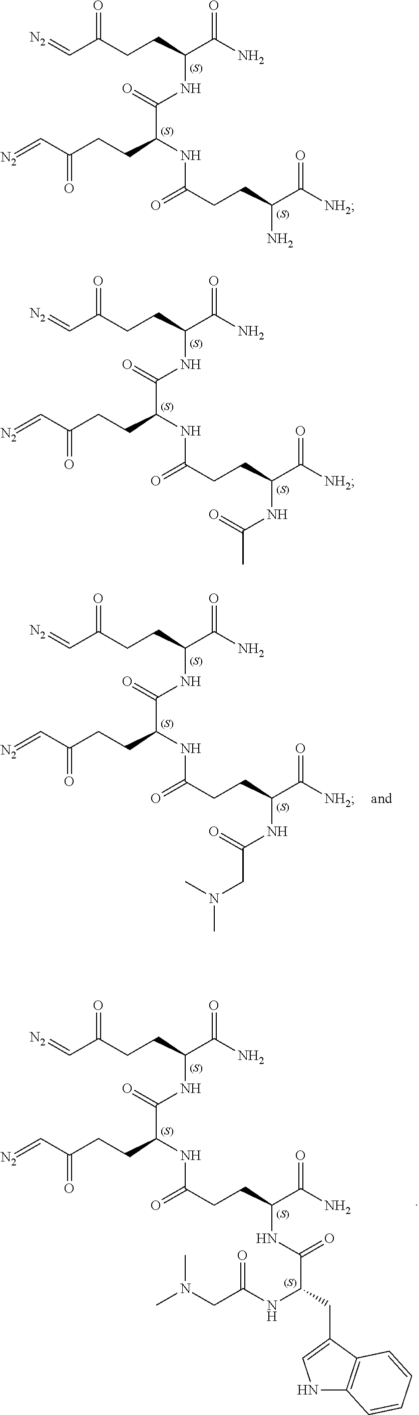

15. The compound of claim 1, wherein at least one of R₁ and R₂ is —O⁻M⁺.

16. The compound of claim 15, wherein the compound is selected from:

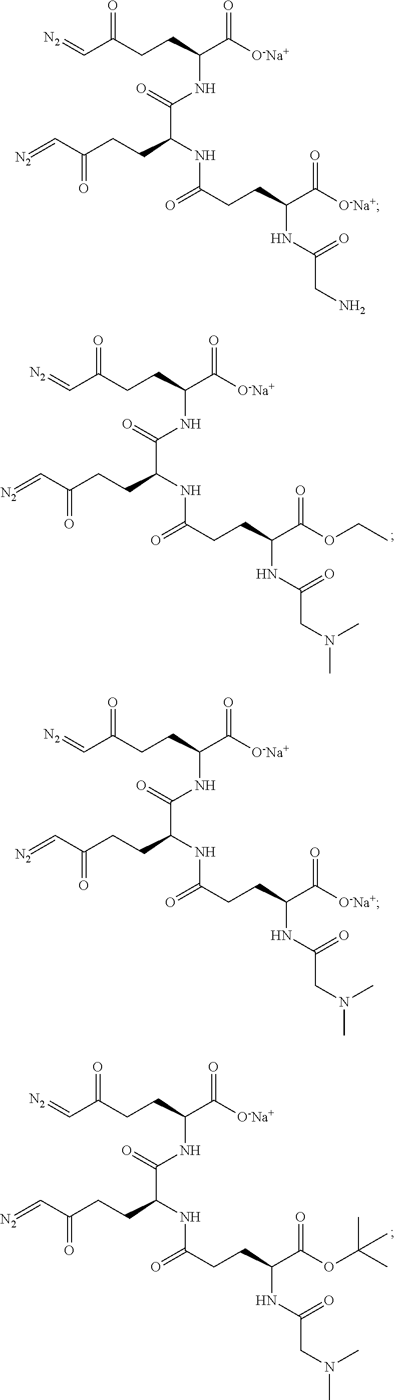

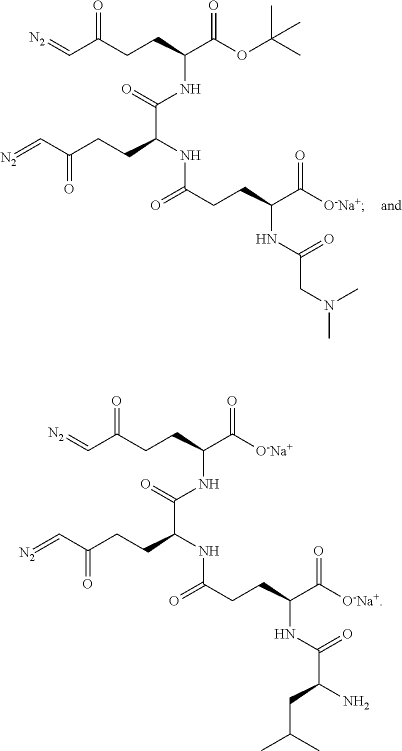

17. The compound of claim 1, wherein the compound is selected from: 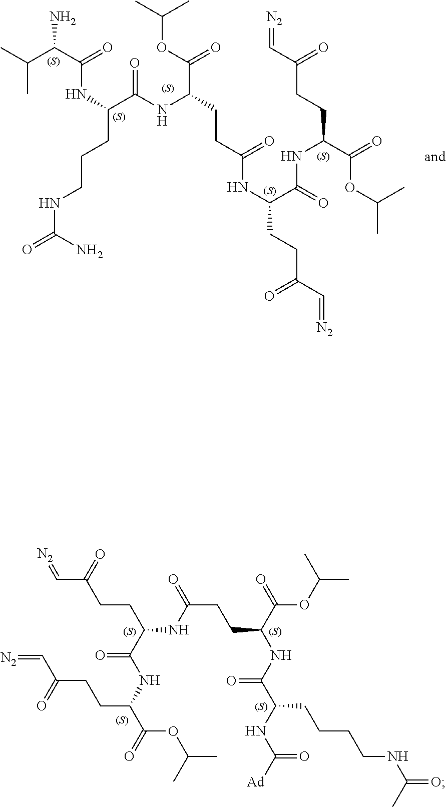
wherein Ad is adamantane.

18. A formulation comprising a compound of claim 1 and a pharmaceutically acceptable carrier.

19. A method for treating a disease, disorder, or condition associated with excess and/or aberrant glutamine utilization, the method comprising administering to a subject in need of treatment thereof, a therapeutically effective amount of a compound of claim 1 or a formulation thereof.

20. The method of claim 19, wherein the disease, disorder, or condition associated with excess and/or aberrant glutamine utilization is selected from an infection, cancer, an autoimmune disease, an inflammatory disease, and a neurodegenerative or neurological disease.

21. The method of claim 20, wherein the cancer is selected from a newly diagnosed cancer, a recurrent cancer, a refractory cancer, and combinations thereof.

22. The method of claim 20, wherein the cancer is selected from: (i) a cancer of the central nervous system; (ii) a cancer that is associated with transplant and/or immunosuppression; (iii) a cancer that is refractory to chemotherapy; (iv) a cancer that is refractory to photodynanic therapy; (v) a cancer that is refractory to proton therapy; (vi) a cancer that is refractory to radiotherapy; and (vii) a cancer that is refractory to surgery.

23. The method of claim 20, wherein the cancer is selected from celnasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, lung cancer, testicular cancer, Hodgkin's disease, non-Hodgkin's lymphoma, oral cancer, skin cancer, mesothelioma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

24. The method of claim 21, further comprising preventing a relapse in a cancer subject in remission.

25. The method of claim 19, further comprising administering a therapeutically effective amount of a compound of formula (I) in combination with an immunotherapy.

26. The method of claim 25, wherein the immunotherapy includes a checkpoint blockade therapy.

27. The method of claim 26, wherein the method comprises administering a compound of formula (I) in combination with one or more checkpoint inhibitors.

28. The method of claim 27, wherein the one or more checkpoint inhibitors are selected from anti-PD-1 Ab, anti-PD-L1 Ab, anti-CTLA-4 Ab, anti-TIGIT Ab, and combinations thereof.
